(19) United States
(12) Patent Application Publication
(10) Pub. No.: US 2022/0333162 A1
(43) Pub. Date: Oct. 20, 2022

(54) DETECTION OF CHLAMYDIA TRACHOMATIS NUCLEIC ACID VARIANTS

(71) Applicant: Gen-Probe Incorporated, San Diego, CA (US)

(72) Inventors: Craig B. Clark, San Diego, CA (US); Damon K. Getman, Poway, CA (US); Mehrdad R. Majlessi, Escondido, CA (US); Marion Walcher, Carlsbad, CA (US)

(21) Appl. No.: 17/640,740

(22) PCT Filed: Sep. 3, 2020
(86) PCT No.: PCT/US2020/049277

§ 371 Date: Mar. 4, 2022

Related U.S. Application Data
(60) Provisional application No. 62/704,833, filed on May 29, 2020.
(60) Provisional application No. 62/896,472, filed on Sep. 5, 2019.

(51) Int. Cl. C12Q 1/689

(52) U.S. Cl. CPC C12Q 1/689; C12Q 2600/156

(57) ABSTRACT

Hybridization probe reagents that specifically detect nucleic acids of C. trachomatis, including wildtype and/or variant sequences identified as FI-nvCT C1515T (SEQ ID NO: 17), JP-nvCT C1522T (SEQ ID NO: 12), US-nvCT G1526A (SEQ ID NO:22), and NO-nvCT G1523A (SEQ ID NO:27). Certain probes include nucleotide analogs to enhance desirable binding properties.

### RELATED APPLICATIONS

This application claims the benefit of U.S. Provisional Application Nos. 62/896,472, filed Sep. 5, 2019; and 62/704,833, filed May 29, 2020. The entire disclosures of these prior applications are hereby incorporated by reference.

### TECHNICAL FIELD

The present disclosure generally relates to the field of biotechnology. More particularly, the disclosure concerns molecular diagnostic assays for detecting nucleic acids of Chlamydia trachomatis.

### BACKGROUND

Genetic variants of Chlamydia trachomatis have begun to arise and escape detection after many years of effective nucleic acid-based diagnostic screening. Ribosomal nucleic acids (e.g., encoding 16S or 23S rRNAs) are common targets used in diagnostic assays because these sequences are evolutionarily very stable due to an intimate structure-function relationship. Indeed, the biological function of rRNAs requires a precisely folded structure that must remain stable. A mutation or base change in one part of the molecule generally requires a corresponding change in a different part of the molecule to retain the needed secondary structure. The chance of two complementary mutations occurring simultaneously is very remote.

Recently identified variants in the rRNA sequence of C. trachomatis occurring in different parts of the world have, in some instances, been able to elude detection in some nucleic acid-based assays. The present disclosure addresses detection of these escape mutants.

### SUMMARY OF THE DISCLOSURE

In a first aspect, the disclosure relates to a probe reagent for detecting wildtype and variant C. trachomatis target nucleic acids. The probe reagent generally includes a first oligonucleotide probe having (i) a backbone, (ii) a sequence of bases attached to the backbone, the sequence of bases including SEQ ID NO:66 or the complement thereof allowing for substitution of RNA and DNA equivalent bases, and (iii) a label covalently attached to the backbone by a non-nucleotide linker. The label can produce a detectable signal if the first oligonucleotide probe hybridizes to a wildtype C. trachomatis nucleic acid sequence selected from the group consisting of SEQ ID NO:5 and SEQ ID NO:10, or the complements thereof allowing for substitution of RNA and DNA equivalent bases. Further, the label can produce a detectable signal if the first oligonucleotide probe hybridizes to a variant C. trachomatis nucleic acid sequence selected from the group consisting of SEQ ID NO:20, SEQ ID NO:30, SEQ ID NO:15, and SEQ ID NO:25, or the complements of any of these sequences allowing for substitution of RNA and DNA equivalent bases. In some embodiments, the first oligonucleotide probe is up to 24 bases in length, and the non-nucleotide linker is attached to the backbone between base positions 11 and 12 of SEQ ID NO:66. In some embodiments, the sequence of bases attached to the backbone of the first oligonucleotide probe is selected from the group consisting of SEQ ID NO:84, SEQ ID NO:85, SEQ ID NO:86, SEQ ID NO:87, SEQ ID NO:88, SEQ ID NO:76, and SEQ ID NO:73. In some embodiments, the sequence of bases attached to the backbone of the first oligonucleotide probe is SEQ ID NO:84 with the non-nucleotide linker being attached to the backbone between base positions 12 and 13. In some embodiments, the sequence of bases attached to the backbone of the first oligonucleotide probe is SEQ ID NO:85 with the non-nucleotide linker being attached to the backbone between base positions 13 and 14. In some embodiments, the sequence of bases attached to the backbone of the first oligonucleotide probe is SEQ ID NO:86 with the non-nucleotide linker being attached to the backbone between base positions 12 and 13. In some embodiments, the sequence of bases attached to the backbone of the first oligonucleotide probe is SEQ ID NO:87 with the non-nucleotide linker being attached to the backbone between base positions 11 and 12. In some embodiments, the sequence of bases attached to the backbone of the first oligonucleotide probe is SEQ ID NO:88 with the non-nucleotide linker being attached to the backbone between base positions 12 and 13. In some embodiments, the sequence of bases attached to the backbone of the first oligonucleotide probe is SEQ ID NO:76 with the non-nucleotide linker being attached to the backbone between base positions 13 and 14. In some embodiments, the sequence of bases attached to the backbone of the first oligonucleotide probe is SEQ ID NO:73 with the non-nucleotide linker being attached to the backbone between base positions 13 and 14. In some embodiments, the label of the first oligonucleotide probe includes a chemiluminescent label. In some embodiments, the chemiluminescent label is an acridinium ester. In some embodiments, the backbone of the first oligonucleotide probe includes one or more 2′-methoxy chemical groups. In some embodiments, the probe reagent further including a second oligonucleotide probe, where the second oligonucleotide probe includes a base sequence complementary to 23S ribosomal nucleic acid of C. trachomatis, or the complement thereof, and further includes a label covalently attached thereto, where the label of the second oligonucleotide probe produces a detectable signal if the second oligonucleotide probe is hybridized to a wildtype C. trachomatis nucleic acid sequence including SEQ ID NO:6, or the complement thereof allowing for substitution of RNA and DNA equivalent bases, and where the label of the second oligonucleotide probe does not produce a detectable signal if the second oligonucleotide probe is hybridized to a nucleic acid sequence selected from the group consisting of SEQ ID NO:21, SEQ ID NO:31, SEQ ID NO:16, and SEQ ID NO:26, or the complements thereof allowing for substitution of RNA and DNA equivalent bases. In some embodiments, the second oligonucleotide probe includes a DNA backbone. In some embodiments, the label of the first oligonucleotide probe is the same as the label of the second oligonucleotide probe. In some embodiments, the base sequence of the second oligonucleotide probe is SEQ ID NO:38.

In another aspect, the disclosure relates to a probe reagent for detecting wildtype and variant C. trachomatis target nucleic acids. Generally speaking, the probe reagent can include a first oligonucleotide probe having (i) a backbone, (ii) a sequence of bases attached to the backbone, the sequence of bases including SEQ ID NO:66 or the complement thereof allowing for substitution of RNA and DNA equivalent bases, and (iii) a label covalently attached to the backbone by a non-nucleotide linker. The label produces a detectable signal if the oligonucleotide probe hybridizes to a target nucleic acid sequence of SEQ ID NO:2 or the complement thereof allowing for substitution of RNA and DNA equivalent bases. The label can produce a detectable signal if the oligonucleotide probe hybridizes to a target nucleic acid sequence of SEQ ID NO:7 or the complement thereof allowing for substitution of RNA and DNA equivalent bases. The label can produce a detectable signal if the oligonucleotide probe hybridizes to a target nucleic acid sequence of SEQ ID NO: 17 or the complement thereof allowing for substitution of RNA and DNA equivalent bases. The label can produce a detectable signal if the oligonucleotide probe hybridizes to a target nucleic acid sequence of SEQ ID NO:27 or the complement thereof allowing for substitution of RNA and DNA equivalent bases. The label can produce a detectable signal if the oligonucleotide probe hybridizes to a target nucleic acid sequence of SEQ ID NO:12 or the complement thereof allowing for substitution of RNA and DNA equivalent bases. Further, the label can produce a detectable signal if the oligonucleotide probe hybridizes to a target nucleic acid sequence of SEQ ID NO:22 or the complement thereof allowing for substitution of RNA and DNA equivalent bases. In some embodiments, the first oligonucleotide probe is up to 24 bases in length, and the non-nucleotide linker is attached to the backbone between base positions 11 and 12 of SEQ ID NO:66. In some embodiments, the sequence of bases attached to the backbone of the first oligonucleotide probe is selected from the group consisting of SEQ ID NO:84, SEQ ID NO:85, SEQ ID NO:86, SEQ ID NO:87, SEQ ID NO:88, SEQ ID NO:76, and SEQ ID NO:73. In some embodiments, the sequence of bases attached to the backbone of the first oligonucleotide probe is SEQ ID NO:84 with the non-nucleotide linker being attached to the backbone between base positions 12 and 13. In some embodiments, the sequence of bases attached to the backbone of the first oligonucleotide probe is SEQ ID NO:85 with the non-nucleotide linker being attached to the backbone between base positions 13 and 14. In some embodiments, the sequence of bases attached to the backbone of the first oligonucleotide probe is SEQ ID NO:86 with the non-nucleotide linker being attached to the backbone between base positions 12 and 13. In some embodiments, the sequence of bases attached to the backbone of the first oligonucleotide probe is SEQ ID NO:87 with the non-nucleotide linker being attached to the backbone between base positions 11 and 12. In some embodiments, the sequence of bases attached to the backbone of the first oligonucleotide probe is SEQ ID NO:88 with the non-nucleotide linker being attached to the backbone between base positions 12 and 13. In some embodiments, the sequence of bases attached to the backbone of the first oligonucleotide probe is SEQ ID NO:76 with the non-nucleotide linker being attached to the backbone between base positions 13 and 14. In some embodiments, the sequence of bases attached to the backbone of the first oligonucleotide probe is SEQ ID NO:73 with the non-nucleotide linker being attached to the backbone between base positions 13 and 14. In some embodiments, the label of the first oligonucleotide probe includes a chemiluminescent label. In some embodiments, the chemiluminescent label is an acridinium ester. In some embodiments, the backbone of the first oligonucleotide probe includes one or more 2′-methoxy chemical groups. In some embodiments, the probe reagent further includes a second oligonucleotide probe, where the second oligonucleotide probe includes a base sequence complementary to 23S ribosomal nucleic acid of wildtype C. trachomatis, or the complement thereof, and further includes a label covalently attached thereto. The label of the second oligonucleotide probe can be positioned to produce a detectable signal if the second oligonucleotide probe is hybridized to a wildtype C. trachomatis nucleic acid sequence including SEQ ID NO:6 or the complement thereof allowing for substitution of RNA and DNA equivalent bases. The label of the second oligonucleotide probe does not produce a detectable signal if the second oligonucleotide probe is hybridized to nucleic acid of any of a variant C. trachomatis nucleic acid sequence of SEQ ID NO:17, SEQ ID NO:27, SEQ ID NO:12, or SEQ ID NO:22 or the complements of these sequences allowing for substitution of RNA and DNA equivalent bases. In some embodiments, the second oligonucleotide probe includes a DNA backbone. In some embodiments, the label of the first oligonucleotide probe is the same as the label of the second oligonucleotide probe. In some embodiments, the base sequence of the second oligonucleotide probe is SEQ ID NO:38.

In another aspect, the disclosure relates to a kit for detecting a 23S ribosomal nucleic acid of wildtype and variant C. trachomatis. Generally speaking, the kit can include a packaged combination of one or more vials containing: a first oligonucleotide probe that produces a detectable signal if hybridized to the wildtype C. trachomatis sequence of SEQ ID NO:6, or the complement thereof allowing for substitution of RNA and DNA equivalent bases, but does not produce a detectable signal if hybridized to variant C. trachomatis nucleic acid sequences of any of SEQ ID NO:14, SEQ ID NO:19, SEQ ID NO:24, and SEQ ID NO:29, or the complements thereof allowing for substitution of RNA and DNA equivalent bases; and a second oligonucleotide probe that produces a detectable signal if hybridized to nucleic acid sequences of any of SEQ ID NO:4, SEQ ID NO:9, SEQ ID NO:14, SEQ ID NO:19, SEQ ID NO:24, and SEQ ID NO:29, or the complements thereof allowing for substitution of RNA and DNA equivalent bases. In some embodiments, the first oligonucleotide probe does not produce a detectable signal if hybridized to a nucleic acid including the sequence of any of SEQ ID NO:16, SEQ ID NO:21, SEQ ID NO:26, and SEQ ID NO:31, and the second oligonucleotide probe produces a detectable signal if hybridized to a nucleic acid including the sequence of any of SEQ ID NO:6, SEQ ID NO:11, SEQ ID NO:16, SEQ ID NO:21, SEQ ID NO:26, and SEQ ID NO:31. In some embodiments, the kit further includes a promoter-primer having an upstream promoter and a downstream target-hybridizing sequence, where the downstream target-hybridizing sequence is complementary to 23S ribosomal nucleic acid of wildtype C. trachomatis sequences of SEQ ID NO:2 and SEQ ID NO:7, and complementary to variant C. trachomatis sequences of SEQ ID NO:12, SEQ ID NO: 17, SEQ ID NO:22, and SEQ ID NO:27, and where enzymatic extension of the promoter-primer using the nucleic acid of either SEQ ID NO:2 or SEQ ID NO:7 as templates produces extension products including sequences complementary to each of the first and second oligonucleotide probes. In some embodiments, each of the first and second oligonucleotide probes includes a backbone and a label covalently attached to the backbone by a non-nucleotide linker. In some embodiments, the backbone of one of the first and second oligonucleotide probes includes at least one 2′-methoxy chemical group. In some embodiments, the backbone of the first oligonucleotide probe includes DNA, and the backbone of the second oligonucleotide probe includes at least one 2′-methoxy chemical group. In some embodiments, the second oligonucleotide probe includes a sequence of bases attached to the backbone thereof, the sequence of bases including SEQ ID NO:66, or the complement thereof allowing for substitution of RNA and DNA equivalent bases, and the non-nucleotide linker is attached to the backbone of the second oligonucleotide probe between base positions 11 and 12 of SEQ ID NO:66. In some embodiments, the sequence of bases of the second oligonucleotide probe is selected from the group consisting of SEQ ID NO:84, SEQ ID NO:85, SEQ ID NO:86, SEQ ID NO:87, SEQ ID NO:88, SEQ ID NO:76, and SEQ ID NO:73. In some embodiments, the sequence of bases attached to the backbone of the second oligonucleotide probe is SEQ ID NO:84 with the non-nucleotide linker being attached to the backbone between base positions 12 and 13. In some embodiments, the sequence of bases attached to the backbone of the second oligonucleotide probe is SEQ ID NO:85 with the non-nucleotide linker being attached to the backbone between base positions 13 and 14. In some embodiments, the sequence of bases attached to the backbone of the second oligonucleotide probe is SEQ ID NO:86 with the non-nucleotide linker being attached to the backbone between base positions 12 and 13. In some embodiments, the sequence of bases attached to the backbone of the second oligonucleotide probe is SEQ ID NO:87 with the non-nucleotide linker being attached to the backbone between base positions 11 and 12. In some embodiments, the sequence of bases attached to the backbone of the second oligonucleotide probe is SEQ ID NO:88 with the non-nucleotide linker being attached to the backbone between base positions 12 and 13. In some embodiments, the sequence of bases attached to the backbone of the second oligonucleotide probe is SEQ ID NO:76 with the non-nucleotide linker being attached to the backbone between base positions 13 and 14. In some embodiments, the sequence of bases attached to the backbone of the second oligonucleotide probe is SEQ ID NO:73 with the non-nucleotide linker being attached to the backbone between base positions 13 and 14. In some embodiments, the label of each of the first and second oligonucleotide probes is the same as the other. In some embodiments, the label of each of the first and second oligonucleotide probes includes a chemiluminescent label. In some embodiments, the chemiluminescent label attached to each of the first and second oligonucleotide probes includes the same chemiluminescent label. In some embodiments, the chemiluminescent label attached to each of the first and second oligonucleotide probes includes an acridinium ester.

In another aspect, the disclosure relates to a kit for detecting a 23S ribosomal nucleic acid of wildtype and variant C. trachomatis. Generally speaking, the kit can include in packaged combination: a first oligonucleotide probe, and a promoter-primer that includes an upstream promoter and a downstream target-hybridizing sequence. The first oligonucleotide probe includes a base sequence selected from the group consisting of SEQ ID NO:84, SEQ ID NO:85, SEQ ID NO:86, SEQ ID NO:87, SEQ ID NO:88, SEQ ID NO:76, SEQ ID NO:73, or the complements thereof allowing for substitution of RNA and DNA equivalent bases. The first oligonucleotide probe can further include a label covalently attached thereto. The label can produce a detectable signal if the first oligonucleotide probe is hybridized to (i) a wildtype C. trachomatis nucleic acid complementary to either SEQ ID NO:3 or SEQ ID NO:8, allowing for substitution of RNA and DNA equivalent bases, or (ii) a variant C. trachomatis sequence complementary to any of SEQ ID NO: 18, SEQ ID NO:28, SEQ ID NO: 13, and SEQ ID NO:23, allowing for substitution of RNA and DNA equivalent bases. The downstream target-hybridizing sequence of the promoter-primer can be complementary to 23S ribosomal nucleic acid of C. trachomatis, and enzymatic extension of the promoter-primer using 23S ribosomal nucleic acid of wildtype or variant C. trachomatis as templates can produce extension products including sequences complementary to the first oligonucleotide probe. In some embodiments, the first oligonucleotide probe includes a backbone with one or more 2′-methoxy chemical groups. In some embodiments, the base sequence of the first oligonucleotide probe is selected from the group consisting of SEQ ID NO:84, SEQ ID NO:85, SEQ ID NO:86, SEQ ID NO:87, SEQ ID NO:88, SEQ ID NO:76, and SEQ ID NO:73. In some embodiments, the kit further includes a second oligonucleotide probe, where the second oligonucleotide probe includes a label covalently attached thereto, and where the label of the second oligonucleotide probe produces a detectable signal if the second oligonucleotide probe hybridizes to a wildtype C. trachomatis nucleic acid of SEQ ID NO:3 or SEQ ID NO:8, or the complements thereof allowing for substitution of RNA and DNA equivalent bases, and where the label of the second oligonucleotide probe does not produce a detectable signal if the second oligonucleotide probe is hybridized to a nucleic acid sequence selected from the group consisting of SEQ ID NO:18, SEQ ID NO:28, SEQ ID NO:13, and SEQ ID NO:23, or the complements thereof allowing for substitution of RNA and DNA equivalent bases. In some embodiments, the label of each of the first and second oligonucleotide probes is a chemiluminescent label. In some embodiments, the chemiluminescent label of each of the first and second oligonucleotide probes is an acridinium ester. In some embodiments, the chemiluminescent labels of the first and second oligonucleotide probes are the same acridinium ester. In some embodiments, the kit further includes a reverse transcriptase and an RNA polymerase. In some embodiments, the label of the first oligonucleotide probe is covalently attached to the backbone by a non-nucleotide linker. In some embodiments, the base sequence of the first oligonucleotide probe is SEQ ID NO:84 with the non-nucleotide linker being attached to the backbone between base positions 12 and 13. In some embodiments, the base sequence of the first oligonucleotide probe is SEQ ID NO:85 with the non-nucleotide linker being attached to the backbone between base positions 13 and 14. In some embodiments, the base sequence of the first oligonucleotide probe is SEQ ID NO:86 with the non-nucleotide linker being attached to the backbone between base positions 12 and 13. In some embodiments, the base sequence of the first oligonucleotide probe is SEQ ID NO:87 with the non-nucleotide linker being attached to the backbone between base positions 11 and 12. In some embodiments, the base sequence of the first oligonucleotide probe is SEQ ID NO:88 with the non-nucleotide linker being attached to the backbone between base positions 12 and 13. In some embodiments, the base sequence of the first oligonucleotide probe is SEQ ID NO:76 with the non-nucleotide linker being attached to the backbone between base positions 13 and 14. In some embodiments, the base sequence of the first oligonucleotide probe is SEQ ID NO:73 with the non-nucleotide linker being attached to the backbone between base positions 13 and 14.

In another aspect, the disclosure relates to a probe reagent for detecting 23S ribosomal nucleic acid of wildtype and variant C. trachomatis. Generally speaking, the probe reagent includes a first oligonucleotide probe having (i) a backbone including one or more 2′-methoxy chemical groups, (ii) a sequence of bases attached to the backbone, the sequence of bases including SEQ ID NO:58, or the complement thereof allowing for substitution of RNA and DNA equivalent bases, and (iii) a label covalently attached to the backbone by a non-nucleotide linker either between base positions 6 and 7, base positions 8 and 9, or base positions 9 and 10 of the sequence of SEQ ID NO:58. The label of the first oligonucleotide probe can produce a detectable signal if the first oligonucleotide probe hybridizes to a target nucleic acid sequence of SEQ ID NO:6, or the complement thereof allowing for substitution of RNA and DNA equivalent bases. The label of the first oligonucleotide probe can produce a detectable signal if the first oligonucleotide probe hybridizes to a target nucleic acid sequence of SEQ ID NO:21, or the complement thereof allowing for substitution of RNA and DNA equivalent bases. The label of the first oligonucleotide probe can produce a detectable signal if the first oligonucleotide probe hybridizes to a target nucleic acid sequence of SEQ ID NO:31, or the complement thereof allowing for substitution of RNA and DNA equivalent bases. The label of the first oligonucleotide probe can produce a detectable signal if the first oligonucleotide probe hybridizes to a target nucleic acid sequence of SEQ ID NO:16, or the complement thereof allowing for substitution of RNA and DNA equivalent bases. As well, the label of the first oligonucleotide probe can produce a detectable signal if the first oligonucleotide probe hybridizes to a target nucleic acid sequence of SEQ ID NO:26, or the complement thereof allowing for substitution of RNA and DNA equivalent bases. In some embodiments, the sequence of bases attached to the backbone of the first oligonucleotide probe is selected from the group consisting of SEQ ID NO:59 with the non-nucleotide linker attached between base positions 9 and 10, SEQ ID NO:60 with the non-nucleotide linker attached between base positions 10 and 11, SEQ ID NO:61 with the non-nucleotide linker attached between base positions 8 and 9, SEQ ID NO:62 with the non-nucleotide linker attached between base positions 9 and 10, SEQ ID NO:63 with the non-nucleotide linker attached between base positions 10 and 11, SEQ ID NO:64 with the non-nucleotide linker attached between base positions 11 and 12, and SEQ ID NO:65 with the non-nucleotide linker attached between base positions 12 and 13. In some embodiments, the sequence of bases attached to the backbone of the first oligonucleotide probe is SEQ ID NO:60. In some embodiments, the label includes a chemiluminescent label. In some embodiments, the chemiluminescent label is an acridinium ester. In some embodiments, the probe reagent further includes a second oligonucleotide probe that detects 23S ribosomal nucleic acid of wildtype C. trachomatis. In some embodiments, the second oligonucleotide includes a label that is the same as the label joined to the backbone of the first oligonucleotide probe. In some embodiments, wildtype C. trachomatis nucleic acids that can be detected include SEQ ID NO:2 and SEQ ID NO:7, and variant C. trachomatis nucleic acids that can be detected include SEQ ID NO:12, SEQ ID NO:17, SEQ ID NO:22, and SEQ ID NO:27.

In another aspect, the disclosure relates to a probe reagent for detecting the nucleic acid of a variant C. trachomatis. The probe reagent can include: a first oligonucleotide probe having (i) a backbone, (ii) a sequence of bases attached to the backbone, the sequence of bases including SEQ ID NO:39, or the complement thereof allowing for substitution of RNA and DNA equivalent bases, and (iii) a label covalently attached to the backbone by a non-nucleotide linker between base positions 6 and 7 of SEQ ID NO:39. The label can produce a detectable signal if the first oligonucleotide probe hybridizes to the nucleic acid sequence of SEQ ID NO: 18, or the complement thereof allowing for substitution of RNA and DNA equivalent bases. The label does not produce the detectable signal if the first oligonucleotide probe hybridizes to the nucleic acid sequence of either SEQ ID NO:3 or SEQ ID NO:8, or the complements of these sequences allowing for substitution of RNA and DNA equivalent bases. In some embodiments, the backbone of the first oligonucleotide probe includes DNA. In some embodiments, the sequence of bases of the first oligonucleotide probe includes SEQ ID NO:39, where the label produces the detectable signal if the first oligonucleotide probe hybridizes to nucleic acid complementary to the sequence of SEQ ID NO:18, allowing for substitution of RNA and DNA equivalent bases, and where the label does not produce the detectable signal if the first oligonucleotide probe hybridizes to the nucleic acid sequence complementary to the sequence of either SEQ ID NO:3 or SEQ ID NO:8. In some embodiments, the base sequence of the first oligonucleotide probe is selected from the group consisting of SEQ ID NO:43, SEQ ID NO:54, and SEQ ID NO:45. In some embodiments, the probe reagent further includes a second oligonucleotide probe that produces a detectable signal if the second oligonucleotide probe hybridizes to the wildtype C. trachomatis nucleic acid sequence complementary to the sequence of either SEQ ID NO:3 or SEQ ID NO:8. In some embodiments, the label of the first oligonucleotide probe includes a chemiluminescent label. In some embodiments, the chemiluminescent label is an acridinium ester. In some embodiments, the base sequence of the first oligonucleotide probe is SEQ ID NO:43 with the non-nucleotide linker being attached to the backbone between base positions 7 and 8. In some embodiments, the base sequence of the first oligonucleotide probe is SEQ ID NO:54 with the non-nucleotide linker being attached to the backbone between base positions 6 and 7. In some embodiments, the base sequence of the first oligonucleotide probe is SEQ ID NO:45 with the non-nucleotide linker being attached to the backbone between base positions 7 and 8.

In another aspect, the disclosure relates to a reaction mixture for detecting C. trachomatis nucleic acids that may be present in a test sample. Generally speaking, the reaction mixture can include a nucleic acid amplification product produced from a 23S ribosomal nucleic acid template of a wildtype or a variant C. trachomatis; and one or more detectably labeled hybridization probes, each of the detectably labeled hybridization probes hybridizing to the nucleic acid amplification product, at least one of the detectably labeled hybridization probes including 2′-methoxy nucleotide analogs, and at least one of the detectably labeled hybridization probes producing a detectable signal after hybridizing to the nucleic acid amplification product. In some embodiments, the variant C. trachomatis nucleic acids that are detectable include the sequences of SEQ ID NO: 17, SEQ ID NO:12, SEQ ID NO:22, and SEQ ID NO:27.

### BRIEF DESCRIPTION OF THE DRAWINGS

FIG. 1 shows aligned segments of the Chlamydia trachomatis 23S ribosomal nucleic acid from seven different isolates. The isolates are: wildtype (“Chlamydia trachomatis E/Bour” (SEQ ID NO:1)); wildtype WT (SEQ ID NO:2); wildtype WT-A (SEQ ID NO:7); JP-nvCT C1522T (SEQ ID NO:12); FI-nvCT C1515T (SEQ ID NO:17); US-nvCT G1526A (SEQ ID NO:22); and NO-nvCT G1523A (SEQ ID NO:27). Single nucleotide differences relative to the wildtype sequence are illustrated.

FIG. 2 is a plot showing relative light unit (RLU) signals on the vertical axis, with the horizontal axis indicating different probes (identified as A-Q) hybridized to one of four panels. The panels are products of TMA amplification reactions performed using the following templates: Mut (FI-nvCT C1515T (SEQ ID NO:17)); STM (specimen transport medium); WT (wildtype C. trachomatis (SEQ ID NO:2)); and the combination of the WT and Mut templates. Probes referenced in the figure are: A (SEQ ID NO:40); B (SEQ ID NO:41); C (SEQ ID NO:42); D (SEQ ID NO:43); E (SEQ ID NO:44); F (SEQ ID NO:45); G (SEQ ID NO:46); H (SEQ ID NO:47); I (SEQ ID NO:48); J (SEQ ID NO:49); K (SEQ ID NO:50); L (SEQ ID NO:51); M (SEQ ID NO:52); N (SEQ ID NO:53); O (SEQ ID NO:54); P (SEQ ID NO:55); and Q (SEQ ID NO:56).

FIG. 3 is a bar graph showing results from processing the data from FIG. 2 to determine ratios of the Mut hybridization signal to the wildtype hybridization signal for probes A-Q (horizontal axis). Higher ratios reflect greater probe specificity for the Mut target. Probes referenced in the figure are: A (SEQ ID NO:40); B (SEQ ID NO:41); C (SEQ ID NO:42); D (SEQ ID NO:43); E (SEQ ID NO:44); F (SEQ ID NO:45); G (SEQ ID NO:46); H (SEQ ID NO:47); I (SEQ ID NO:48); J (SEQ ID NO:49); K (SEQ ID NO:50); L (SEQ ID NO:51); M (SEQ ID NO:52); N (SEQ ID NO:53); O (SEQ ID NO:54); P (SEQ ID NO:55); and Q (SEQ ID NO:56).

FIG. 4 is a plot showing relative light unit (RLU) signals for different amplified nucleic acid samples (templates being either FI-nvCT C1515T (SEQ ID NO:17) or wildtype C. trachomatis (SEQ ID NO:2) nucleic acids to produce in vitro transcripts for use in amplification reactions) hybridized to different probes (either 346978 (SEQ ID NO:38), or the combination of 350078 (SEQ ID NO:57) and 346978 (SEQ ID NO:38)) that detect C. trachomatis ribosomal nucleic acid sequences.

FIGS. 5A and 5B are plots showing relative light unit (RLU) signals (vertical axis) for different hybridization reactions. FIG. 5A shows results for hybridization of the methoxy backbone probe of 350116.1 (SEQ ID NO:59) to different targets. These were: a pooled lysate of potentially cross-hybridizing non-Chlamydia trachomatis species (i.e., Chlamydia pneumoniae and Chlamydia psittaci); FI-nvCT C1515T (SEQ ID NO:17) IVT at input levels of 1×10³ or 3×10³ copies; US-nvCT G1526A (SEQ ID NO:22) IVT at input levels of 1×10³ or 3×10³ copies; wildtype (SEQ ID NO:2) IVT at input levels of 1×10³ or 3×10³ copies; and an STM negative control (i.e., no input template). FIG. 5B shows results for hybridization of the methoxy probe of 350547 (SEQ ID NO:60) to lysates of potentially cross-hybridizing non-Chlamydia trachomatis species (i.e., Chlamydia pneumoniae (C. pne) and Chlamydia psittaci (C. psi)), each being used at 1×10⁵ CFU/ml; FI-nvCT C1515T (SEQ ID NO: 17) IVT at 3×10³ copies/ml (CT_MutB); US-nvCT G1526A (SEQ ID NO:22) IVT at 3×10³ copies/ml (CT_MutC); JP-nvCT C1522T (SEQ ID NO:12) IVT at 3×10³ copies/ml (CT_MutD); NO-nvCT G1523A (SEQ ID NO:27) IVT at 3×10³ copies/ml (CT_MutE); wildtype (SEQ ID NO:2) IVT at 3×10³ copies/ml (CT_WT); wildtype strain A (SEQ ID NO:7) IVT at 3×10³ copies/ml (CT_WT-A); and an STM negative control (i.e., no input template).

FIG. 6 is a plot showing RLU signal (vertical axis) for hybridization of different probes for three different target conditions. The target conditions were represented by products of TMA reactions using as template sources: Chlamydia pneumoniae lysate, Chlamydia psittaci lysate, or wildtype IVT. Probes used in the procedure were 350502 or “502” (SEQ ID NO:71), 350506 or “506” (SEQ ID NO:72), 350507 or “507” (SEQ ID NO:73), 350509 or “509” (SEQ ID NO:74), 350510 or “510” (SEQ ID NO:75), 350511 or “511” (SEQ ID NO:76), 350563 or “563” (SEQ ID NO:80), 350565 or “565” (SEQ ID NO:79), 350566 or “566” (SEQ ID NO:78), 350567 or “567” (SEQ ID NO:77), 350568 or “568” (SEQ ID NO:81), 350570 or “570” (SEQ ID NO:82), 350571 or “571” (SEQ ID NO:83), 350600 or “600” (SEQ ID NO:88), 350601 or “601” (SEQ ID NO:87), 350609 or “609” (SEQ ID NO:86), 350610 or “610” (SEQ ID NO:85), 350611 or “611” (SEQ ID NO:84), and 346978 or “978” (SEQ ID NO:38).

### DETAILED DESCRIPTION

### Introduction and Overview

Disclosed herein are compositions, methods, and kits for selectively detecting the nucleic acids of C. trachomatis in a biological sample. Probes in accordance with the disclosure can be used either in diagnostic applications or for screening of samples that may contain this bacterium. In some embodiments, the disclosed probes can be used for detecting a particular C. trachomatis variant that harbors a genetic difference relative to a wildtype in a sequence encoding the 23S rRNA. In other embodiments, the disclosed probes can be used for detecting wildtype and variant sequences encoding the 23S rRNA.

Those having an ordinary level of skill in the art will appreciate that standardized workflows frequently impose constraints on the reagents and methods used for amplifying and detecting C. trachomatis nucleic acids. For example, preselected hybridization temperatures may determine the length and melting temperature (i.e., “Tm”) profiles of useful probes. As detailed below, the structure of labeled probes profoundly influenced the ability to detect genetic differences in amplified nucleic acid products.

Below there are described different approaches that can be used to detect nucleic acids of wildtype C. trachomatis, one or more variants of C. trachomatis, or the combination of wildtype and one or more variants of C. trachomatis.

### Definitions

The following terms have the indicated meanings in the specification unless expressly indicated to have a different meaning.

The terms “a,” “an,” and “the” include plural referents, unless the context clearly indicates otherwise. For example, “a nucleic acid” as used herein is understood to represent one or more nucleic acids. As such, the terms “a” (or “an”), “one or more,” and “at least one” can be used interchangeably herein.

By “sample” or “test sample” is meant any substance suspected of containing a target organism or nucleic acid derived from the target organism. The substance may be, for example, an unprocessed clinical specimen, such as a sputum or urethral specimen, a buffered medium containing the specimen, a medium containing the specimen and lytic agents for releasing nucleic acid belonging to the target organism, or a medium containing nucleic acid derived from the target organism which has been isolated and/or purified in a reaction receptacle or on a reaction material or device. In the claims, the terms “sample” and “test sample” may refer to specimen in its raw form or to any stage of processing to release, isolate and purify nucleic acid derived from target organisms in the specimen.

By “target nucleic acid” or “target” is meant a nucleic acid containing a target nucleic acid sequence.

By “target nucleic acid sequence,” “target nucleotide sequence,” “target sequence” or “target region” is meant a specific deoxyribonucleotide or ribonucleotide sequence comprising all or part of the nucleotide sequence of a single-stranded nucleic acid molecule, and the deoxyribonucleotide or ribonucleotide sequence complementary thereto. The claims, however, may restrict a target sequence to the particular sense of the recited sequence with a proviso excluding complementary sequences thereof.

“Nucleic acid” and “polynucleotide” refer to a multimeric compound comprising nucleosides or nucleoside analogs which have nitrogenous heterocyclic bases or base analogs linked together to form a polynucleotide, including conventional RNA, DNA, mixed RNA-DNA, and polymers that are analogs thereof. A nucleic acid “backbone” may be made up of a variety of linkages, including one or more of sugar-phosphodiester linkages, peptide-nucleic acid bonds (“peptide nucleic acids” or PNA; PCT Publication No. WO 95/32305), phosphorothioate linkages, methylphosphonate linkages, or combinations thereof. Sugar moieties of a nucleic acid may be ribose, deoxyribose, or similar compounds with substitutions (e.g., 2′ methoxy or 2′ halide substitutions). Nitrogenous bases may be conventional bases (A, G, C, T, U), analogs thereof (e.g., inosine or others; see The Biochemistry of the Nucleic Acids 5-36, Adams et al., ed., 11^{th} ed., 1992), derivatives of purines or pyrimidines (e.g., N⁴-methyl deoxyguanosine, deaza- or aza-purines, deaza- or aza-pyrimidines, pyrimidine bases with substituent groups at the 5 or 6 position, purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT Publication No. WO 93/13121). Nucleic acids may include one or more “abasic” residues where the backbone includes no nitrogenous base for position(s) of the polymer (U.S. Pat. No. 5,585,481). A nucleic acid may comprise only conventional RNA or DNA sugars, bases and linkages, or may include both conventional components and substitutions (e.g., conventional bases with 2′ methoxy backbones, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). Embodiments of oligomers that may affect stability of a hybridization complex include PNA oligomers, oligomers that include 2′-methoxy or 2′-fluoro substituted RNA, or oligomers that affect the overall charge, charge density, or steric associations of a hybridization complex, including oligomers that contain charged linkages (e.g., phosphorothioates) or neutral groups (e.g., methylphosphonates). 5-methylcytosines may be used in conjunction with any of the foregoing backbones/sugars/linkages including RNA or DNA backbones (or mixtures thereof) unless otherwise indicated. It is understood that when referring to ranges for the length of an oligonucleotide, amplicon, or other nucleic acid, that the range is inclusive of all whole numbers (e.g., 19-25 contiguous nucleotides in length includes 19, 20, 21, 22, 23, 24, and 25).

A “nucleotide” as used herein is a subunit of a nucleic acid consisting of a phosphate group, a 5-carbon sugar, and a nitrogenous base (also referred to herein as “nucleobase”). The 5-carbon sugar found in RNA is ribose. In DNA, the 5-carbon sugar is 2′-deoxyribose. The term also includes analogs of such subunits, such as a methoxy group at the 2′ position of the ribose (also referred to herein alternatively as “2′-O-methyl” or “2′-O-Me” or “2′-methoxy” or “2′-OMe”).

“Oligomer,” “oligonucleotide,” or “oligo” refers to a nucleic acid of generally less than 1,000 nucleotides (nt), including those in a size range having a lower limit of about 2 to 5 nucleotides and an upper limit of about 500 to 900 nucleotides. Some particular embodiments are oligomers in a size range with a lower limit of about 18, 19, 20, 21, 22, 23, 24, or 25 nucleotides and an upper limit of about 50 to 60 nucleotides, and other particular embodiments are in a size range with a lower limit of about 10 to 20 nucleotides and an upper limit of about 22 to 100 nucleotides. Oligomers may be purified from naturally occurring sources, but may be synthesized by using any well-known enzymatic or chemical method. The term oligonucleotide does not denote any particular function of the reagent; rather, it is used generically to cover all such reagents described herein. An oligonucleotide may serve various different functions. For example, it may function as a primer if it is specific for and capable of hybridizing to a complementary strand and can further be extended in the presence of a nucleic acid polymerase; it may function as a primer and provide a promoter if it contains a sequence recognized by an RNA polymerase and allows for transcription (e.g., a T7 Primer); and it may function to detect a target nucleic acid if it is capable of hybridizing to the target nucleic acid, or an amplicon thereof, and further provides a detectible moiety (e.g., an acridinium-ester compound). Oligomers may be referred to by a functional name (e.g., capture probe, primer or promoter primer) but those skilled in the art will understand that such terms refer to oligomers.

Oligonucleotides of a defined sequence may be produced by techniques known to those of ordinary skill in the art, such as by chemical or biochemical synthesis, and by in vitro or in vivo expression from recombinant nucleic acid molecules (e.g., bacterial or retroviral vectors). As intended by this disclosure, an oligonucleotide may not consist of wild-type chromosomal DNA or the in vivo transcription products thereof. For example, oligonucleotide hybridization probes can include non-nucleotide linkers and/or detectable labels that are not found in naturally occurring nucleic acids.

“Detection probe oligomer,” “detection probe,” or “probe” refers to an oligomer that hybridizes specifically to a target sequence, including an amplified sequence, under conditions that promote nucleic acid hybridization, for detection of the target nucleic acid. Detection may either be direct (i.e., probe hybridized directly to the target) or indirect (i.e., a probe hybridized to an intermediate structure that links the probe to the target). Detection probes may be DNA, RNA, analogs thereof or combinations thereof (e.g., DNA/RNA chimerics), and they may be labeled or unlabeled. Detection probes may further include alternative backbone linkages (e.g., 2′-O-methyl linkages). A probe's target sequence generally refers to the specific sequence within a larger sequence which the probe hybridizes specifically. A detection probe may include target-specific sequence(s) and non-target-specific sequence(s). Such non-target-specific sequences can include sequences which will confer a desired secondary or tertiary structure, such as a hairpin structure, which can be used to facilitate detection and/or amplification (see, e.g., U.S. Pat. Nos. 5,118,801, 5,312,728, 6,835,542, and 6,849,412). Probes of a defined sequence may be produced by techniques known to those of ordinary skill in the art, such as by chemical synthesis, and by in vitro or in vivo expression from recombinant nucleic acid molecules.

As used herein, a “probe reagent” is a composition that includes one or more probes. In some embodiments, probes of the reagent are oligonucleotide probes, optionally including a detectable label (e.g., a label that can be detected using optical means, such as a luminometer or a fluorometer). Example probe reagents include one or more detectably labeled oligonucleotide probes.

“Label” or “detectable label” refers to a moiety or compound joined directly or indirectly to a probe that is detected or leads to a detectable signal. Direct joining may use covalent bonds or non-covalent interactions (e.g., hydrogen bonding, hydrophobic or ionic interactions, and chelate or coordination complex formation) whereas indirect joining may use a bridging moiety or linker (e.g., via an antibody or additional oligonucleotide(s)). Any detectable moiety may be used, including a radionuclide, a ligand such as biotin or avidin or even a polynucleotide sequence, an enzyme, an enzyme substrate, a reactive group, a chromophore such as a dye or particle (e.g., a latex or metal bead) that imparts a detectable color, a luminescent compound (e.g., bioluminescent, phosphorescent, or a chemiluminescent compound), and a fluorescent compound or moiety (i.e., fluorophore). Embodiments of fluorophores include those that absorb light in the range of about 495 to 650 nm and emit light in the range of about 520 to 670 nm, which include those known as FAM™, TET™, CAL FLUOR™ (Orange or Red), and QUASAR™ compounds. Fluorophores may be used in combination with a quencher molecule that absorbs light when in close proximity to the fluorophore to diminish background fluorescence. Such quenchers are well known in the art and include, for example, BLACK HOLE QUENCHER™ (or BHQ™) or TAMRA™ compounds. Particular embodiments include a “homogeneous detectable label” that is detectable in a homogeneous system in which bound labeled probe in a mixture exhibits a detectable change compared to unbound labeled probe, which allows the label to be detected without physically removing hybridized from unhybridized labeled probe (e.g., U.S. Pat. Nos. 5,283,174, 5,656,207, and 5,658,737). Particular homogeneous detectable labels include chemiluminescent compounds, including acridinium ester (“AE”) compounds, such as standard AE or AE derivatives which are well known (U.S. Pat. Nos. 5,656,207, 5,658,737, and 5,639,604). Methods of synthesizing labels, attaching labels to nucleic acid, and detecting signals from labels are well known (e.g., Sambrook et al., Molecular Cloning, A Laboratory Manual, 2nd ed. (Cold Spring Harbor Laboratory Press, Cold Spring Harbor, N.Y., 1989) at Chapt. 10, and U.S. Pat. Nos. 5,658,737, 5,656,207, 5,547,842, 5,283,174, and 4,581,333, and EP Pat. App. 0 747 706). Particular methods of linking an AE compound to a nucleic acid are known (e.g., U.S. Pat. Nos. 5,585,481 and 5,639,604, see column 10, line 6 to column 11, line 3, and Example 8). Particular AE labeling positions are a probe's central region and near a region of A/T base pairs, at a probe's 3′ or 5′ terminus, or at or near a mismatch site with a known sequence that is the probe should not detect compared to the desired target sequence. Other detectably labeled probes include TaqMan™ probes, molecular torches, and molecular beacons. TaqMan™ probes include a donor and acceptor label wherein fluorescence is detected upon enzymatically degrading the probe during amplification in order to release the fluorophore from the presence of the quencher. Molecular torches and beacons exist in open and closed configurations wherein the closed configuration quenches the fluorophore and the open position separates the fluorophore from the quencher to allow fluorescence. Hybridization to a target nucleic acid opens the otherwise closed probes.

By “stably,” “stable” or “stable for detection” is meant that the temperature of a reaction mixture is at least 2° C. below the melting temperature of a nucleic acid duplex. The temperature of the reaction mixture is more preferably at least 5° C. below the melting temperature of the nucleic acid duplex, and even more preferably at least 10° C. below the melting temperature of the reaction mixture.

By “substantially homologous,” “substantially corresponding” or “substantially corresponds” is meant that the subject oligonucleotide has a base sequence containing an at least 10 contiguous base region that is at least about 80% homologous, preferably at least about 90% homologous, and most preferably 100% homologous to an at least 10 contiguous base region present in a reference base sequence (excluding RNA and DNA equivalents). (Those skilled in the art will readily appreciate modifications that could be made to the hybridization assay conditions at various percentages of homology to permit hybridization of the oligonucleotide to the target sequence while preventing levels of non-specific hybridization sufficient to interfere with detection of the target nucleic acid.) The degree of similarity is determined by comparing the order of nucleobases making up the two sequences and does not take into consideration other structural differences which may exist between the two sequences, provided the structural differences do not prevent hydrogen bonding with complementary bases. The degree of homology between two sequences can also be expressed in terms of the number of base differences between each set of at least 10 contiguous bases being compared, which may be 0, 1 or 2 base differences.

By “substantially complementary” is meant that the subject oligonucleotide has abase sequence containing an at least 10 contiguous base region that is at least 80% complementary, preferably at least 90% complementary, and most preferably 100% complementary to an at least 10 contiguous base region present in a target nucleic acid sequence (excluding RNA and DNA equivalents). Those skilled in the art will readily appreciate modifications that could be made to the hybridization assay conditions at various percentages of complementarity to permit hybridization of the oligonucleotide to the target sequence while preventing levels of non-specific hybridization sufficient to interfere with detection of the target nucleic acid. The degree of complementarity is determined by comparing the order of nucleobases making up the two sequences and does not take into consideration other structural differences which may exist between the two sequences, provided the structural differences do not prevent hydrogen bonding with complementary bases. The degree of complementarity between two sequences can also be expressed in terms of the number of base mismatches present in each set of at least 10 contiguous bases being compared, which may be 0, 1 or 2 base mismatches.

It will be appreciated that there is an implied “about” prior to the temperatures, concentrations, and times discussed in the present disclosure, such that slight and insubstantial deviations are within the scope of the present teachings. In general, the term “about” indicates insubstantial variation in a quantity of a component of a composition not having any significant effect on the activity or stability of the composition. All ranges are to be interpreted as encompassing the endpoints in the absence of express exclusions such as “not including the endpoints”; thus, for example, “within 10-15” includes the values 10 and 15. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

By “RNA and DNA equivalents” is meant RNA and DNA molecules having essentially the same complementary base pair hybridization properties. RNA and DNA equivalents have different sugar moieties (i.e., ribose versus deoxyribose) and may differ by the presence of uracil in RNA and thymine in DNA. The differences between RNA and DNA equivalents do not contribute to differences in homology because the equivalents have the same degree of complementarity to a particular sequence.

By “RNA and DNA equivalent bases” is meant nucleotide bases having the same complementary base pair hybridization properties in RNA and DNA. Here the base uracil can be substituted in place of the base thymine, or vice versa, and so uracil and thymine are RNA and DNA equivalent bases. A polynucleotide base sequence 5′-AGCT-3′ that allows for substitution of RNA and DNA equivalent bases would also describe the sequence 5′-AGCU-3′. The differences between RNA and DNA equivalent bases do not contribute to differences in homology because the equivalents have the same degree of complementarity to a particular sequence.

The term, “complement” refers to a nucleic acid molecule that comprises a contiguous nucleic acid sequence that is complementary to a contiguous nucleic acid sequence of another nucleic acid molecule (for standard nucleotides A:T, A:U, C:G). For example, 5′-AACTGUC-3′ is the complement of 5′-GACAGTT-3′.

A “target nucleic acid” as used herein is a nucleic acid comprising a target sequence to be amplified. Target nucleic acids may be DNA or RNA, and may be either single-stranded or double-stranded. The target nucleic acid may include other sequences besides the target sequence, which may not be amplified.

The term “target sequence” as used herein refers to the particular nucleotide sequence of the target nucleic acid that is to be amplified and/or detected. The “target sequence” includes the complexing sequences to which oligonucleotides (e.g., priming oligonucleotides and/or promoter oligonucleotides) complex during an amplification processes (e.g., PCR, TMA). Where the target nucleic acid is originally single-stranded, the term “target sequence” will also refer to the sequence complementary to the “target sequence” as present in the target nucleic acid. Where the target nucleic acid is originally double-stranded, the term “target sequence” refers to both the sense (+) and antisense (−) strands.

“Target-hybridizing sequence of bases” or “target-hybridizing sequence” or “target-specific sequence” is used herein to refer to the portion of an oligomer that is configured to hybridize with a target nucleic acid sequence. Preferably, the target-hybridizing sequences are configured to specifically hybridize with a target nucleic acid sequence. Target-hybridizing sequences may be 100% complementary to the portion of the target sequence to which they are configured to hybridize, but not necessarily. Target-hybridizing sequences may also include inserted, deleted and/or substituted nucleotide residues relative to a target sequence. Less than 100% complementarity of a target-hybridizing sequence to a target sequence may arise, for example, when the target nucleic acid is a plurality strains within a species, such as would be the case for an oligomer configured to hybridize to sequence variants. It is understood that other reasons exist for configuring a target-hybridizing sequence to have less than 100% complementarity to a target nucleic acid.

By “hybridization” or “hybridize” is meant the ability of two completely or partially complementary nucleic acid strands to come together under specified hybridization assay conditions in a parallel or antiparallel orientation to form a stable structure having a double-stranded region. The two constituent strands of this double-stranded structure, sometimes called a hybrid, are held together by hydrogen bonds. Although these hydrogen bonds most commonly form between nucleotides containing the bases adenine and thymine or uracil (A and T or U) or cytosine and guanine (C and G) on single nucleic acid strands, base pairing can also form between bases which are not members of these “canonical” pairs. Non-canonical base pairing is well-known in the art. See, e.g., R. L. P. Adams et al., The Biochemistry of the Nucleic Acids (11th ed. 1992).

By “preferentially hybridize” is meant that under stringent hybridization assay conditions, hybridization assay probes can hybridize to their target nucleic acids to form stable probe:target hybrids indicating the presence of at least one organism of interest (“detectable hybrids”), and there is not formed a sufficient number of detectable stable probe:non-target hybrids to indicate the presence of non-targeted organisms (“non-detectable hybrids”), especially phylogenetically closely related organisms. Thus, the probe hybridizes to target nucleic acid to a sufficiently greater extent than to non-target nucleic acid to enable one having ordinary skill in the art to accurately detect the presence (or absence) of nucleic acid derived from C. trachomatis, and distinguish its presence from that of a phylogenetically closely related organism in a test sample. In general, reducing the degree of complementarity between an oligonucleotide sequence and its target sequence will decrease the degree or rate of hybridization of the oligonucleotide to its target region. However, the inclusion of one or more non-complementary bases may facilitate the ability of an oligonucleotide to discriminate against non-target organisms.

Preferential hybridization can be measured using any of a variety of techniques known in the art, including, but not limited to those based on light emission, mass changes, and changes in conductivity or turbidity. A number of detection means are described herein, and one in particular is used in the Examples provided below. Preferably, there is at least a 10-fold difference between target and non-target hybridization signals in a test sample, more preferably at least a 100-fold difference, and most preferably at least a 500-fold difference. Preferably, non-target hybridization signals in a test sample are no more than the background signal level.

By “stringent hybridization assay conditions,” “hybridization assay conditions,” “stringent hybridization conditions,” or “stringent conditions” is meant conditions permitting a hybridization assay probe to preferentially hybridize to a target nucleic acid (preferably rRNA or rDNA derived from C. trachomatis over nucleic acid derived from a closely related non-target microorganism. Stringent hybridization assay conditions may vary depending upon factors including the GC content and length of the probe, the degree of similarity between the probe sequence and sequences of non-target sequences which may be present in the test sample, and the target sequence. Hybridization conditions include the temperature and the composition of the hybridization reagents or solutions. While the Examples section infra provides preferred hybridization assay conditions for detecting target nucleic acids derived from C. trachomatis using the probes of the present disclosure, other stringent conditions could be easily ascertained by someone having ordinary skill in the art.

By “assay conditions” is meant conditions permitting stable hybridization of an oligonucleotide to a target nucleic acid. Assay conditions do not require preferential hybridization of the oligonucleotide to the target nucleic acid.

A “homogeneous detectable label” refers to a label that can be detected in a homogeneous fashion by determining whether the label is on a probe hybridized to a target sequence. That is, homogeneous detectable labels can be detected without physically removing hybridized from unhybridized forms of the label or labeled probe. Homogeneous detectable labels are preferred when using labeled probes for detecting amplified nucleic acids. Examples of homogeneous labels have been described in detail by Arnold et al., U.S. Pat. No. 5,283,174; Woodhead et al., U.S. Pat. No. 5,656,207; and Nelson et al., U.S. Pat. No. 5,658,737. Preferred labels for use in homogenous assays include chemiluminescent compounds (e.g., see Woodhead et al., U.S. Pat. No. 5,656,207; Nelson et al., U.S. Pat. No. 5,658,737; and Arnold, Jr., et al., U.S. Pat. No. 5,639,604). Preferred chemiluminescent labels are acridinium ester (“AE”) compounds, such as standard AE or derivatives thereof (e.g., naphthyl-AE, ortho-AE, 1- or 3-methyl-AE, 2,7-dimethyl-AE, 4,5-dimethyl-AE, ortho-dibromo-AE, ortho-dimethyl-AE, meta-dimethyl-AE, ortho-methoxy-AE, ortho-methoxy(cinnamyl)-AE, ortho-methyl-AE, ortho-fluoro-AE, 1- or 3-methyl-ortho-fluoro-AE, 1- or 3-methyl-meta-difluoro-AE, and 2-methyl-AE).

A “homogeneous assay” refers to a detection procedure that does not require physical separation of hybridized probe from non-hybridized probe prior to determining the extent of specific probe hybridization. Exemplary homogeneous assays, such as those described herein, can employ molecular beacons or other self-reporting probes which emit fluorescent signals when hybridized to an appropriate target, chemiluminescent acridinium ester labels which can be selectively destroyed by chemical means unless present in a hybrid duplex, and other homogeneously detectable labels that will be familiar to those having an ordinary level of skill in the art.

By “consisting essentially of” is meant that additional component(s), composition(s) or method step(s) that do not materially change the basic and novel characteristics of the present invention may be included in the compositions or kits or methods of the present invention. Any component(s), composition(s), or method step(s) that have a material effect on the basic and novel characteristics of the present invention would fall outside of this term. For example, additions or deletions to an oligonucleotide can be non-material variations which do not prevent the oligonucleotide from having its claimed property (i.e., preferentially hybridizing under stringent hybridization assay conditions to the target nucleic acid over non-target nucleic acids). The oligonucleotide may include other nucleic acid molecules which do not participate in hybridization of the probe to the target nucleic acid and which do not affect such hybridization.

By “nucleic acid duplex,” “duplex,” “nucleic acid hybrid” or “hybrid” is meant a stable nucleic acid structure comprising a double-stranded, hydrogen-bonded region. Such hybrids include RNA:RNA, RNA:DNA and DNA:DNA duplex molecules and analogs thereof. The structure is sufficiently stable to be detectable by any known means.

An “amplification oligonucleotide” or “amplification oligomer” is an oligonucleotide that hybridizes to a target nucleic acid, or its complement, and participates in a nucleic acid amplification reaction (e.g., serving as a primer or promoter-primer). Particular amplification oligomers contain at least about 10 contiguous bases, and optionally at least 18, 19, 20, 21, 22, or 23 contiguous bases that are complementary to a region of the target nucleic acid sequence or its complementary strand. The contiguous bases may be at least about 80%, at least about 90%, or completely complementary to the target sequence to which the amplification oligomer binds. One skilled in the art will understand that the recited ranges include all whole and rational numbers within the range (e.g., 92% or 98.377%). Particular amplification oligomers are about 10 to about 60 bases long, or more preferably about 18 to about 26 bases long and optionally may include modified nucleotides.

A “primer” is an oligomer that hybridizes to a template nucleic acid and has a 3′ end that is extended by a polymerase enzyme. A primer may be optionally modified, e.g., by including a 5′ region that is non-complementary to the target sequence. Such modification can include functional additions, such as tags, promoters, or other non-target-specific sequences used or useful for manipulating or amplifying the primer or target oligonucleotide.

Within the context of transcription-mediated amplification, a primer modified with a 5′ promoter sequence is referred to herein as a “promoter-primer.” A person of ordinary skill in the art of molecular biology or biochemistry will understand that an oligomer that can function as a primer can be modified to include a 5′ promoter sequence and then function as a promoter-primer, and, similarly, any promoter-primer can serve as a primer with or without its 5′ promoter sequence. A promoter-primer modified to incorporate a 3′ blocked end is referred to herein as a “promoter provider,” which is capable of hybridizing to a target nucleic acid and providing an upstream promoter sequence that serves to initiate transcription, but does not provide a primer for oligo extension.

“Nucleic acid amplification” or “target amplification” or simply “amplification” refers to any in vitro procedure that produces multiple copies of a target nucleic acid sequence, or its complementary sequence, or fragments thereof (i.e., an amplified sequence containing less than the complete target nucleic acid). Examples of nucleic acid amplification procedures include transcription associated methods, such as transcription-mediated amplification (TMA), nucleic acid sequence-based amplification (NASBA) and others (e.g., U.S. Pat. Nos. 5,399,491, 5,554,516, 5,437,990, 5,130,238, 4,868,105, and 5,124,246), replicase-mediated amplification (e.g., U.S. Pat. No. 4,786,600), the polymerase chain reaction (PCR) (e.g., U.S. Pat. Nos. 4,683,195, 4,683,202, and 4,800,159), ligase chain reaction (LCR) (e.g., EP Patent No. 0320308), helicase-dependent amplification (e.g., U.S. Pat. No. 7,282,328), and strand-displacement amplification (SDA) (e.g., U.S. Pat. No. 5,422,252). Amplification may be linear or exponential. PCR amplification uses DNA polymerase, primers, and thermal cycling steps to synthesize multiple copies of the two complementary strands of DNA or cDNA. LCR amplification uses at least four separate oligonucleotides to amplify a target and its complementary strand by using multiple cycles of hybridization, ligation, and denaturation. Helicase-dependent amplification uses a helicase to separate the two strands of a DNA duplex generating single-stranded templates, followed by hybridization of sequence-specific primers hybridize to the templates and extension by DNA polymerase to amplify the target sequence. SDA uses a primer that contains a recognition site for a restriction endonuclease that will nick one strand of a hemimodified DNA duplex that includes the target sequence, followed by amplification in a series of primer extension and strand displacement steps. Replicase-mediated amplification uses self-replicating RNA molecules, and a replicase such as QB-replicase. Particular embodiments use PCR or TMA, but it will be apparent to persons of ordinary skill in the art that oligomers disclosed herein may be readily used as primers in other amplification methods.

Transcription-associated amplification uses a DNA polymerase, an RNA polymerase, deoxyribonucleoside triphosphates, ribonucleoside triphosphates, a promoter-containing oligonucleotide, and optionally may include other oligonucleotides, to ultimately produce multiple RNA transcripts from a nucleic acid template (described in detail in, e.g., U.S. Pat. Nos. 5,399,491 and 5,554,516 to Kacian et al.; U.S. Pat. No. 5,437,990 to Burg et al.; PCT Publication Nos. WO 88/01302 and WO 88/10315 (Gingeras et al.); U.S. Pat. No. 5,130,238 to Malek et al.; U.S. Pat. Nos. 4,868,105 and 5,124,246 to Urdea et al.; PCT Publication No. WO 94/03472 (McDonough et al.); and PCT Publication No. WO 95/03430 (Ryder et al.)). Methods that use TMA are described in detail previously (e.g., U.S. Pat. Nos. 5,399,491 and 5,554,516).

By “amplification conditions” is meant conditions permitting nucleic acid amplification. While the Examples section infra provides preferred amplification conditions for amplifying target nucleic acid sequences derived from Chlamydia organisms using primers of the present disclosure in a transcription-mediated amplification method, other acceptable amplification conditions could be easily determined by one having ordinary skill in the art, depending on the particular method of amplification desired.

By “opposite sense” or “opposite strand” is meant a nucleic acid molecule perfectly complementary to a reference, or sense, nucleic acid strand.

By “sense,” “same-sense” or “positive sense” is meant a nucleic acid molecule perfectly homologous to a reference nucleic acid molecule.

By “amplicon” is meant a nucleic acid molecule generated in a nucleic acid amplification reaction and which is derived from a target nucleic acid. An amplicon contains a target nucleic acid sequence that may be of the same or opposite sense as the target nucleic acid.

### Hybridization Conditions and Probe Design

Hybridization reaction conditions, most importantly the temperature of hybridization and the concentration of salt in the hybridization solution, can be selected to allow the hybridization assay probes of the present disclosure to preferentially hybridize to nucleic acids having a target nucleic sequence derived from C. trachomatis. At decreased salt concentrations and/or increased temperatures (conditions of increased stringency) the extent of nucleic acid hybridization decreases as hydrogen bonding between paired nucleotide bases in the double-stranded hybrid molecule is disrupted. This process is known as “melting.”

Generally speaking, the most stable hybrids are those having the largest number of contiguous, perfectly matched (i.e., hydrogen-bonded) nucleotide base pairs. Such hybrids would usually be expected to be the last to melt as the stringency of the hybridization conditions increases. However, a double-stranded nucleic acid region containing one or more mismatched, “non-canonical,” or imperfect base pairs (resulting in weaker or non-existent base pairing at that position in the nucleotide sequence of a nucleic acid) may still be sufficiently stable under conditions of relatively high stringency to allow the nucleic acid hybrid to be formed and detected in a hybridization assay without cross-reacting with other, non-selected nucleic acids which may be present in a test sample.

Hence, depending on the degree of similarity between the nucleotide sequences of the target nucleic acid and those of non-target nucleic acids belonging to phylogenetically distinct, but closely related organisms on the one hand, and the degree of complementarity between the nucleotide sequences of a particular probe and those of the target and non-target nucleic acids on the other, one or more mismatches will not necessarily defeat the ability of an oligonucleotide contained in the probe or primer to hybridize to the target nucleic acid and not to non-target nucleic acids.

The hybridization assay probes of the present disclosure were chosen, selected, and/or designed to maximize the difference between the melting temperatures Tₘ of the probe:target hybrid (Tₘ is defined as the temperature at which half of the potentially double-stranded molecules in a given reaction mixture are in a single-stranded, denatured state) and the Tₘ of a mismatched hybrid formed between the probe and rRNA or rDNA of the phylogenetically most closely-related organisms expected to be present in the test sample, but not sought to be detected. While the unlabeled amplification primers and capture probes need not have such an extremely high degree of specificity as the hybridization assay probe to be useful in the present disclosure, they are designed in a similar manner to preferentially hybridize to one or more target nucleic acids over other nucleic acids under specified amplification or hybridization assay conditions.

Within the rRNA molecule there is a close relationship between secondary structure (caused in part by intra-molecular hydrogen bonding) and function. This fact imposes restrictions on evolutionary changes in the primary nucleotide sequence causing the secondary structure to be maintained. For example, if a base is changed in one “strand” of a double helix (due to intra-molecular hydrogen bonding, both “strands” are part of the same rRNA molecule), a compensating substitution usually occurs in the primary sequence of the other “strand” in order to preserve complementarity (this is referred to as co-variance), and thus the necessary secondary structure. This allows two very different rRNA sequences to be aligned based both on the conserved primary sequence and also on the conserved secondary structure elements. Potential target sequences for the hybridization assay probes described herein were identified by noting variations in the homology of the aligned sequences.

The sequence evolution at each of the variable regions is mostly divergent. As a result of this divergence, corresponding rRNA variable regions of more distant phylogenetic relatives of C. trachomatis show greater differences from the rRNA of these organisms than do the rRNAs of phylogenetically closer relatives.

Merely identifying putatively unique potential target nucleotide sequences does not guarantee that a functionally species-specific hybridization assay probe may be made to hybridize to C. trachomatis rRNA or rDNA comprising that sequence. Various other factors will determine the suitability of a nucleic acid locus as a target site for species-specific probes. Because the extent and specificity of hybridization reactions, such as those described herein, are affected by a number of factors, manipulation of one or more of those factors will determine the exact sensitivity and specificity of a particular oligonucleotide, whether perfectly complementary to its target or not. The importance and effect of various assay conditions are known to those skilled in the art and are disclosed by the following: Kohne, “Method for Detection, Identification and Quantitation of Non-Viral Organisms,” U.S. Pat. No. 4,851,330; Hogan et al., “Nucleic Acid Probes to Mycobacterium gordonae,” U.S. Pat. No. 5,216,143; and Hogan, “Nucleic Acid Probes for Detection and/or Quantitation of Non-Viral Organisms,” U.S. Pat. No. 5,840,488.

The desired temperature of hybridization and the hybridization solution composition (such as salt concentration, detergents and other solutes) can also affect the stability of double-stranded hybrids. Conditions such as ionic strength and the temperature at which a probe will be allowed to hybridize to a target must be taken into account in constructing a species-specific probe. The thermal stability of hybrid nucleic acids generally increases with the ionic strength of the reaction mixture. On the other hand, chemical reagents which disrupt hydrogen bonds, such as formamide, urea, dimethyl sulfoxide and alcohols, can greatly reduce the thermal stability of the hybrids.

To maximize the specificity of a probe for its target, probes of the present disclosure were designed to hybridize to their targets under conditions of high stringency. Under such conditions only single nucleic acid strands (or regions) having a high degree of complementarity will hybridize to each other. Single nucleic acid strands without such a high degree of complementarity will not form hybrids. Accordingly, the stringency of the assay conditions determines the amount of complementarity which should exist between two nucleic acid strands in order to form a hybrid. Stringency is chosen to maximize the difference in stability between the hybrid formed between the probe and the target nucleic acid and potential hybrids between the probe and any non-target nucleic acids present in a test sample.

Proper specificity may be achieved by minimizing the length of the hybridization assay probe having perfect complementarity to sequences of non-target organisms, by avoiding G and C rich regions of complementarity to non-target nucleic acids, and by constructing the probe to contain as many destabilizing mismatches to non-target sequences as possible. Whether a probe is appropriate for detecting only a specific type of organism depends largely on the thermal stability difference between probe:target hybrids versus probe:non-target hybrids. In designing probes, the differences in these Tₘ values should be as large as possible (preferably 2-5° C. or more). Manipulation of the Tₘ can be accomplished by changes to probe length and probe composition, such as GC content versus AT content or the inclusion of nucleotide analogs (e.g., ribonucleotides having a 2′-O-methyl substitution to the ribofuranosyl moiety).

In general, the optimal hybridization temperature for oligonucleotide probes is approximately 5° C. below the melting temperature for a given duplex. Incubation at temperatures below the optimum temperature may allow mismatched base sequences to hybridize and can therefore decrease specificity. The longer the probe, the more hydrogen bonding between base pairs and, in general, the higher the Tₘ. Increasing the percentage of G and C also increases the Tₘ because G-C base pairs exhibit additional hydrogen bonding and therefore greater thermal stability than A-T base pairs. Such considerations are known in the art. See, e.g., J. SAMBROOK ET AL., MOLECULAR CLONING: A LABORATORY MANUAL CH. 11 (2d ed. 1989).

A preferred method for determining Tₘ measures hybridization using the well-known hybridization protection assay (HPA) disclosed by Arnold et al., “Homogenous Protection Assay,” U.S. Pat. No. 5,283,174. The Tₘ can be measured using HPA in the following manner. Probe molecules are labeled with an acridinium ester and permitted to form probe:target hybrids in a lithium succinate buffer (0.1 M lithium succinate buffer, pH 4.7, 20 mM EDTA, 15 mM aldrithiol-2, 1.2 M LiCl, 3% (v/v) ethanol absolute, 2% (w/v) lithium lauryl sulfate) using an excess amount of target. Aliquots of the solution containing the probe:target hybrids are then diluted in the lithium succinate buffered solution and incubated for five minutes at various temperatures starting below that of the anticipated Tₘ (typically 55° C.) and increasing in 2-5° C. increments. This solution is then diluted with a mild alkaline borate buffer (600 mM boric acid, 240 mM NaOH, 1% (v/v) TRITON® X-100, pH 8.5) and incubated at an equal or lower temperature (for example 50° C.) for ten minutes.

Under these conditions the acridinium ester attached to the single-stranded probe is hydrolyzed, while the acridinium ester attached to hybridized probe is relatively protected from hydrolysis. Thus, the amount of acridinium ester remaining after hydrolysis treatment is proportional to the number of hybrid molecules. The remaining acridinium ester can be measured by monitoring the chemiluminescence produced from the remaining acridinium ester by adding hydrogen peroxide and alkali to the solution. Chemiluminescence can be measured in a luminometer, such as a LEADER® 450i luminometer (Gen-Probe Incorporated, San Diego, Calif.). The resulting data can be plotted as percent of maximum signal (usually from the lowest temperature) versus temperature. The Tₘ is defined as the temperature at which 50% of the maximum signal remains. In addition to the method above, Tₘ may be determined by isotopic methods known to those skilled in the art (see, e.g., U.S. Pat. No. 5,840,488).

It should be noted that the Tₘ for a given hybrid varies depending on the nature of the hybridization solution used. Factors such as the salt concentration, detergents, and other solutes can affect hybrid stability during thermal denaturation (see, e.g., SAMBROOK ET AL., supra, ch. 11). Conditions such as ionic strength and the temperature at which a probe will be allowed to hybridize to target should be taken into account in probe construction. Generally speaking, the thermal stability of a hybrid nucleic acid increases with the ionic strength of the reaction mixture. On the other hand, chemical reagents that disrupt hydrogen bonds, such as formamide, urea, dimethyl sulfoxide and alcohols, can greatly reduce hybrid thermal stability.

To ensure specificity of a hybridization assay probe for its target, it is preferable to design probes which hybridize only to target nucleic acid under conditions of high stringency. Only highly complementary sequences will form hybrids under conditions of high stringency. Accordingly, the stringency of the assay conditions determines the amount of complementarity needed between two sequences in order for a stable hybrid to form. Stringency should be chosen to maximize the difference in stability between the probe:target hybrid and potential probe:non-target hybrids.

Examples of specific stringent hybridization conditions are provided herein. Of course, alternative stringent hybridization conditions could be determined by those of ordinary skill in the art based on the present disclosure. (See, e.g., SAMBROOK ET AL., supra, ch. 11.)

The length of the target nucleic acid sequence region and, accordingly, the length of the probe sequence can also be important. In some cases, there may be several sequences from a particular region, varying in location and length, which may be used to design probes with the desired hybridization characteristics. In other cases, one probe may be significantly better with regard to specificity than another which differs from it merely by a single base. While it is possible for nucleic acids that are not perfectly complementary to hybridize, the longest stretch of perfectly complementary bases, as well as the base compositions, will generally determine hybrid stability.

Regions of rRNA known to form strong internal structures inhibitory to hybridization are less preferred target regions. Likewise, probes with extensive self-complementarity are generally to be avoided. However, that some degree of self-complementarity in a probe may be desirable, as in hairpin probes like the molecular beacons and molecular torches discussed below. If a strand is wholly or partially involved in an intra-molecular or inter-molecular hybrid, it will be less able to participate in the formation of a new inter-molecular probe:target hybrid without a change in the reaction conditions. Ribosomal RNA molecules are known to form very stable intra-molecular helices and secondary structures by hydrogen bonding. By designing a probe to a region of the target nucleic acid which remains substantially single-stranded under hybridization conditions, the rate and extent of hybridization between probe and target may be increased.

A genomic ribosomal nucleic acid (rDNA) target occurs naturally in a double-stranded form, as does a product of the polymerase chain reaction (PCR). These double-stranded targets are naturally inhibitory to hybridization with a probe and require denaturation prior to hybridization. Appropriate denaturation and hybridization conditions are known in the art (see, e.g., Southern, E. M., J. Mol. Biol., 98:503 (1975)).

A number of formulae are available which will provide an estimate of the melting temperature for perfectly matched oligonucleotides to their target nucleic acids. One such formula is the following:

Tₘ=81.5+16.6(log₁₀[Na⁺])+0.41(fraction G+C)−(600/N)

(where N=the length of the oligonucleotide in number of nucleotides) provides a good estimate of the Tₘ for oligonucleotides between 14 and 60 to 70 nucleotides in length. From such calculations, subsequent empirical verification or “fine tuning” of the Tₘ may be made using screening techniques well known in the art. For further information on hybridization and oligonucleotide probes reference, may be made to SAMBROOK ET AL., supra, ch. 11. This reference, among others well known in the art, also provides estimates of the effect of mismatches on the Tₘ of a hybrid. Thus, from the known nucleotide sequence of a given region of the ribosomal RNA (or rDNA) of two or more organisms, oligonucleotides may be designed which will distinguish these organisms from one another.

### Preparation of Oligonucleotides

The hybridization assay probes, amplification primers and capture probes of the present disclosure can be readily prepared by methods known in the art. Preferably, the oligonucleotides are synthesized using solid phase methods. Standard phosphoramidite solid-phase chemistry for joining nucleotides by phosphodiester linkages is disclosed by Caruthers et al., in “Chemical Synthesis of Deoxynucleotides by the Phosphoramidite Method,” Methods Enzymol., 154:287 (1987). Automated solid-phase chemical synthesis using cyanoethyl phosphoramidite precursors has been described by Barone. See Barone et al., “In Situ Activation of bis-dialkylaminephosphines—a New Method for Synthesizing Deoxyoligonucleotides on Polymer Supports,” Nucleic Acids Res., 12(10):4051 (1984). Batt discloses a procedure for synthesizing oligonucleotides containing phosphorothioate linkages in U.S. Pat. No. 5,449,769, entitled “Method and Reagent for Sulfurization of Organophosphorous Compounds.” In addition, Riley et al. disclose the synthesis of oligonucleotides having different linkages including methylphosphonate linkages in U.S. Pat. No. 5,811,538, entitled “Process for the Purification of Oligomers.” Moreover, methods for the organic synthesis of oligonucleotides are known to those of skill in the art and are described in, for example, SAMBROOK ET AL., supra, ch. 10.

Following synthesis and purification of a particular oligonucleotide, several different procedures may be utilized to purify and control the quality of the oligonucleotide. Suitable procedures include polyacrylamide gel electrophoresis or high pressure liquid chromatography. Both of these procedures are well known to those skilled in the art.

All of the oligonucleotides of the present disclosure, whether hybridization assay probes, amplification primers or capture probes, may be modified with chemical groups to enhance their performance or to facilitate the characterization of amplification products. For example, backbone-modified oligonucleotides such as those having phosphorothioate, methylphosphonate, 2′-O-alkyl or peptide groups which render the oligonucleotides resistant to the nucleolytic activity of certain polymerases or to nuclease enzymes may allow the use of such enzymes in an amplification or other reaction. Another example of a modification involves using non-nucleotide linkers incorporated between nucleotides in the nucleic acid chain of a probe or primer, and which do not prevent hybridization of a probe or hybridization and elongation of a primer. See Arnold et al., “Non-Nucleotide Linking Reagents for Nucleotide Probes,” U.S. Pat. No. 6,031,091. The oligonucleotides of the present disclosure may also contain mixtures of the desired modified and natural nucleotides.

The 3′ end of an amplification primer can be modified or blocked to prevent or inhibit initiation of DNA synthesis, as disclosed by Kacian et al. in U.S. Pat. No. 5,554,516. The 3′ end of the primer can be modified in a variety of ways well known in the art. By way of example, appropriate modifications to a primer can include the addition of ribonucleotides, 3′ deoxynucleotide residues (e.g., cordycepin), 2′,3′-dideoxynucleotide residues, modified nucleotides such as phosphorothioates, and non-nucleotide linkages such as those disclosed by Arnold et al. in U.S. Pat. No. 6,031,091 or alkane-diol modifications (see Wilk et al., “Backbone-Modified Oligonucleotides Containing a Butanediol-1,3 Moiety as a ‘Vicarious Segment’ for the Deoxyribosyl Moiety—Synthesis and Enzyme Studies,” Nucleic Acids Res., 18(8):2065 (1990)), or the modification may simply consist of a region 3′ to the priming sequence that is non-complementary to the target nucleic acid sequence. Additionally, a mixture of different 3′ blocked primers or of 3′ blocked and unblocked primers may increase the efficiency of nucleic acid amplification, as disclosed therein.

The 5′ end of primers can be modified to be resistant to the 5′-exonuclease activity present in some nucleic acid polymerases. Such modifications can be carried out by adding a non-nucleotide group to the terminal 5′ nucleotide of the primer using techniques such as those disclosed by Arnold et al. in U.S. Pat. No. 6,031,091. To facilitate strand displacement, the 5′ end may also be modified to include non-complementary nucleotides as disclosed by Dattagupta et al, “Isothermal Strand Displacement Nucleic Acid Amplification,” U.S. Pat. No. 6,087,133.

Once synthesized, a selected oligonucleotide may be labeled by any of several well-known methods (see, e.g., SAMBROOK, supra, ch. 10). Useful labels include radioisotopes as well as non-radioactive reporting groups. Isotopic labels include ³H, ³⁵S, ³²P, ¹²⁵I, ⁵⁷Co and ¹⁴C. Isotopic labels can be introduced into the oligonucleotide by techniques known in the art such as nick translation, end labeling, second strand synthesis, the use of reverse transcription, and by chemical methods. When using radiolabeled probes, hybridization can be detected by autoradiography, scintillation counting or gamma counting. The detection method selected will depend upon the particular radioisotope used for labeling.

Non-isotopic materials can also be used for labeling and may be introduced internally into the nucleic acid sequence or at the end of the nucleic acid sequence. Modified nucleotides may be incorporated enzymatically or chemically. Chemical modifications of the probe may be performed during or after synthesis of the probe, for example, through the use of non-nucleotide linker groups, as disclosed by Arnold et al. in U.S. Pat. No. 6,031,091. Non-isotopic labels include fluorescent molecules (individual labels or combinations of interacting labels, such as the fluorescence resonance energy transfer (FRET) pairs disclosed by Tyagi et al. in U.S. Pat. No. 5,925,517), chemiluminescent molecules, enzymes, cofactors, enzyme substrates, haptens or other ligands. With the hybridization assay probes of the present disclosure, the probes are preferably labeled by means of a non-nucleotide linker with an acridinium ester (AE), such as standard AE. Acridinium ester labeling may be performed as disclosed by Arnold et al., “Acridinium Ester Labelling and Purification of Nucleotide Probes,” U.S. Pat. No. 5,185,439.

### Nucleic Acid Amplification

Preferably, the amplification primers of the present disclosure are oligodeoxynucleotides and are sufficiently long to be used as a substrate for the synthesis of extension products by a nucleic acid polymerase. Optimal primer length should take into account several factors, including the temperature of reaction, the structure and base composition of the primer, and how the primer is to be used. For example, for optimal specificity the oligonucleotide primer generally should be at least 12 bases in length, depending on the complexity of the target nucleic acid sequence. If such specificity is not essential, shorter primers may be used. In such a case, it may be desirable to carry out the reaction at cooler temperatures in order to form stable hybrid complexes with the template nucleic acid.

Useful guidelines for designing amplification primers with desired characteristics are described above in the section entitled “Preparation of Oligonucleotides.” Optimal sites for amplifying and probing contain at least two, and preferably three, conserved regions of C. trachomatis nucleic acid. These regions are about 15 to 350 bases in length, and preferably between about 15 and 150 bases in length.

The degree of amplification observed with a set of amplification primers (primers and/or promoter-primers) depends on several factors, including the ability of the primers to hybridize to their specific target sequences and their ability to be extended or copied enzymatically. While amplification primers of different lengths and base compositions may be used, amplification primers preferred in this disclosure have target binding regions of 18 to 40 bases with a predicted Tₘ to target above 42° C., preferably at least about 50° C.

Parameters affecting probe hybridization, such as melting temperature, complementarity and secondary structure of the target sequence, also affect amplification primer hybridization and therefore performance of the amplification primers. The degree of non-specific extension (primer-dimer or non-target copying) can also affect amplification efficiency. Thus, amplification primers are generally selected to have low self-complementarity or cross-complementarity, particularly at the 3′ ends of their sequences. Notwithstanding, amplification primers including regions of self-complementarity may be useful, such as the self-reporting “signal primers” disclosed by Nadeau et al., “Detection of Nucleic Acids by Fluorescence Quenching,” U.S. Pat. No. 5,958,700, and the “hairpin primers” disclosed by Nazarenko et al., “Nucleic Acid Amplification Oligonucleotides with Molecular Energy Transfer Labels and Methods Based Thereon,” U.S. Pat. No. 5,866,336. Lengthy homopolymer runs and high GC content are avoided to reduce spurious primer extension. Computer programs are available to aid in this aspect of the design, including Oligo Tech® analysis software available from Oligo Therapeutics, Inc.

A nucleic acid polymerase used in conjunction with the amplification primers of the present disclosure refers to a chemical, physical or biological agent which incorporates either ribonucleotides or deoxyribonucleotides, or both, into a nucleic acid polymer, or strand, in a template-dependent manner. Examples of nucleic acid polymerases include DNA-directed DNA polymerases, RNA-directed DNA polymerases, and RNA-directed RNA polymerases. DNA polymerases bring about nucleic acid synthesis in a template-dependent manner and in a 5′ to 3′ direction. Because of the typical anti-parallel orientation of the two strands in a double-stranded nucleic acid, this direction is from a 3′ region on the template to a 5′ region on the template. Examples of DNA-directed DNA polymerases include E. coli DNA polymerase I, the thermostable DNA polymerase from Thermus aquaticus (Taq), and the large fragment of DNA polymerase I from Bacillus stearothermophilus (Bst). See, e.g., Riggs et al., “Purified DNA Polymerase from Bacillus stearothermophilus,” U.S. Pat. No. 6,066,483. Examples of RNA-directed DNA polymerases include various retroviral reverse transcriptases, such as Moloney murine leukemia virus (MMLV) reverse transcriptase or avian myeloblastosis virus (AMV) reverse transcriptase.

During most nucleic acid amplification reactions, a nucleic acid polymerase adds nucleotide residues to the 3′ end of the primer using the target nucleic acid as a template, thus synthesizing a second nucleic acid strand having a nucleotide sequence partially or completely complementary to a region of the target nucleic acid. In many nucleic acid amplification reactions, the two strands comprising the resulting double-stranded structure must be separated by chemical or physical means in order to allow the amplification reaction to proceed. Alternatively, the newly-synthesized template strand may be made available for hybridization with a second primer or promoter-primer by other means, such as through strand displacement or the use of a nucleolytic enzyme which digests part or all of the original target strand. In this way the process may be repeated through a number of cycles, resulting in a large increase in the number of nucleic acid molecules having the target nucleotide sequence. Either the first or second amplification primer, or both, may be a promoter-primer. In some applications, the amplification primers may only consist of promoter-primers which are complementary to the sense strand, as disclosed by Kacian et al., “Nucleic Acid Sequence Amplification Method, Composition and Kit,” U.S. Pat. No. 5,554,516. A promoter-primer usually contains an oligonucleotide segment that is not complementary to a nucleotide sequence present in the target nucleic acid molecule or primer extension product(s) (see, e.g., Kacian et al., “Nucleic Acid Sequence Amplification Methods,” U.S. Pat. No. 5,399,491). These non-complementary sequences may be located 5′ to the complementary sequences on the amplification primer and may provide a locus for initiation of RNA synthesis when made double-stranded through the action of a nucleic acid polymerase. The promoter thus provided may allow for the in vitro transcription of multiple RNA copies of the target nucleic acid sequence. It will be appreciated that all references to primers herein are inclusive of primers and promoter-primers, unless the context clearly indicates otherwise.

### Diagnostic Systems

The present disclosure also contemplates diagnostic systems in kit form. A diagnostic system of the present disclosure may include a kit which contains, in an amount sufficient for at least one assay, any of the hybridization assay probes of the present disclosure, and optionally primers that amplify both wildtype and variant C. trachomatis nucleic acids, in a packaging material. Typically, the kits will also include instructions recorded in a tangible form (e.g., contained on paper or an electronic medium) for using the packaged probes and/or primers in an amplification and/or detection assay for determining the presence or amount of C. trachomatis in a test sample.

The various components of the diagnostic systems may be provided in a variety of forms. For example, the required enzymes, the nucleotide triphosphates, the probes and/or primers may be provided as a lyophilized reagent. These lyophilized reagents may be pre-mixed before lyophilization so that when reconstituted they form a complete mixture with the proper ratio of each of the components ready for use in the assay. In addition, the diagnostic systems of the present disclosure may contain a reconstitution reagent for reconstituting the lyophilized reagents of the kit. In preferred kits for amplifying target nucleic acid derived from C. trachomatis, the enzymes, nucleotide triphosphates and required cofactors for the enzymes are provided as a single lyophilized reagent that, when reconstituted, forms a proper reagent for use in the present amplification methods. In these kits, a lyophilized primer reagent may also be provided. In other preferred kits, lyophilized probe reagents are provided.

Typical packaging materials would include solid matrices such as glass, plastic, paper, foil, micro-particles, and the like, capable of holding within fixed limits hybridization assay probes and/or amplification primers of the present disclosure. Thus, for example, the packaging materials can include glass or plastic vials used to contain sub-milligram (e.g., picogram or nanogram) quantities of a contemplated probe or primer, or they can be microtiter plate wells to which probes or primers of the present disclosure have been operatively affixed, i.e., linked so as to be capable of participating in an amplification and/or detection method of the present disclosure.

The instructions will typically indicate the reagents and/or concentrations of reagents and at least one assay method parameter which might be, for example, the relative amounts of reagents to use per amount of sample. In addition, such specifics as maintenance, time periods, temperature and buffer conditions may also be included. The diagnostic systems of the present disclosure contemplate kits having any of the hybridization assay probes described herein, whether provided individually or in one of the preferred combinations described above, for use in amplifying and/or determining the presence or amount of C. trachomatis in a test sample.

Examples are provided below illustrating different aspects and embodiments of the disclosure. Skilled artisans will appreciate that these examples are not intended to limit the disclosure to the specific embodiments described therein.

### Transcription-Mediated Amplification

Amplification of a target sequence in the following Examples was a transcription-mediated amplification (TMA) procedure disclosed by, for example, Kacian et al. in U.S. Pat. Nos. 5,399,491 and 5,480,784 and by LEE ET AL., supra, ch. 8. TMA is an isothermal amplification procedure which allows for a greater than one billion-fold increase in copy number of the target sequence using reverse transcriptase and RNA polymerase (see Enzyme Reagents below). A TMA reaction involves converting a single-stranded target sequence to a double-stranded DNA intermediate by reverse transcriptase in the presence of a sense primer (sometimes a “second primer” or “non-T7 primer,” herein) and an antisense primer (sometimes “T7 promoter-primer” or “first primer,” herein) having a 5′ RNA polymerase-specific promoter sequence. Included in this DNA intermediate is a double-stranded promoter sequence which is recognized by RNA polymerase and transcribed into hundreds of copies of RNA. Each of these transcribed RNA molecules, in turn, can be converted to a double-stranded DNA intermediate which is used for producing additional RNA. Thus, the TMA reaction proceeds exponentially. The particulars of the TMA reactions used in the following examples are set forth below.

Primers used for illustrating the technique disclosed herein had defined sequences. A “first” primer (e.g., a T7 promoter-primer) included the target-complementary sequence of SEQ ID NO:35 joined downstream of a T7 promoter sequence. In this case, the promoter sequence was the T7 promoter sequence of SEQ ID NO:36 (although alternative promoter sequences can be substituted), so the complete first primer had the sequence of SEQ ID NO:37. Polymerase-dependent extension of the first primer using the C. trachomatis 23S rRNA as a template yielded a primer-extension product complementary to (e.g., hybridizible to) the second primer. In some preferred embodiments, the enzymatic extension product of the first primer can hybridize to a detectably labeled probe to indicate the presence of the C. trachomatis 23S rRNA template. The second primer that participated in the amplification reaction (i.e., the non-T7 primer participating in a TMA reaction) had the sequence of SEQ ID NO:34.

### Hybridization Assay Probes

Featured in the Examples are hybridization assay probes having defined nucleotide sequences and detection specificities. All of the hybridization probes described below were synthesized using standard phosphoramidite chemistry using standard procedures well known in the art. See, e.g., Caruthers et al., Methods in Enzymol., 154:287 (1987). Synthesis was performed using an Expedite™ 8909 Nucleic Acid Synthesizer (Applied Biosystems; Foster City, Calif.). Hybridization probes were also synthesized to include a non-nucleotide linker, as described by Arnold et al. in U.S. Pat. No. 6,031,091, and labeled with a chemiluminescent acridinium ester, as described by Arnold et al. in U.S. Pat. No. 5,185,439. The reactivity and specificity of these probes was demonstrated using a single phase homogeneous assay format, essentially as disclosed by Arnold et al. in U.S. Pat. No. 5,283,174. All probe hybridization results are given in relative light units (RLU), which is a measure of the photons detected by a luminometer.

### Nucleic Acids: Templates, Amplicons, and Probe-Binding Sequences

Template nucleic acids used for preparing amplification products (“amplicons”) that were detected in probe-binding assays corresponded either to wildtype or variant C. trachomatis 23S ribosomal nucleic acid sequences. The sequence of the C. trachomatis E/Bour (SEQ ID NO:1) 23S ribosomal nucleic acid served as a model of the wildtype. Probes used for detection harbored labels that produced detectable signals (e.g., detectable optical signals) after hybridizing to appropriate target nucleic acids (e.g., amplicons).

A single nucleotide difference distinguished two wildtype template sequences that were detected in the below-described procedures. “Wildtype” is used herein to refer to a sequence that is common or prevalent in a population, and that can be distinguished from “variants” having different sequences. There can be more than one “wildtype” sequence common among a population of organisms. In the first instance, the wildtype WT template of (SEQ ID NO:2) included a binding site for a first primer. The extension product of the first primer using the WT template contained a binding site for an opposite-strand primer used in an amplification reaction. The subregion of WT that was detected in the probe hybridization procedure had the sequence of WT(0) (SEQ ID NO:3), or the complement thereof allowing for substitution of RNA and DNA equivalent bases. An exemplary amplicon resulting from use of the WT template in a TMA reaction included the probe-binding sequence of WT(1) (SEQ ID NO:4). Contained within this amplification product was a first region having the sequence of WT(2) (SEQ ID NO:5), detectable using one of the “tandem” probes disclosed herein. As well, a second region contained within WT(1), identified as WT(3) (SEQ ID NO:6), was detectable using, for example, a probe having the sequence of 346978 (SEQ ID NO:38) or 350547 (SEQ ID NO:60). Importantly, detection of wildtype nucleic acid sequences is desirable under some circumstances, but undesirable under other circumstances. Likewise, an alternative wildtype Wildtype A (WT-A) template (SEQ ID NO:7) included a binding site for a first primer. The extension product of the first primer using the WT-A template contained a binding site for the opposite strand primer used in the amplification reaction. The subregion of WT-A that was detected in the probe hybridization procedure had the sequence of WT-A(0) (SEQ ID NO:8), or the complement thereof allowing for substitution of RNA and DNA equivalent bases. An exemplary amplicon resulting from use of the wildtype WT-A template in a TMA reaction included the probe-binding sequence of WT-A(1) (SEQ ID NO:9). Contained within this amplification product was a first region having the sequence of WT-A(2) (SEQ ID NO: 10), detectable using one of the “tandem” probes disclosed herein. As well, a second region contained within WT-A (1), identified as WT-A(3) (SEQ ID NO: 11), was detectable using, for example, a probe having the sequence of 346978 (SEQ ID NO:38) or 350547 (SEQ ID NO:60). Notably, the wildtype sequences of WT(2) and WT-A(2) (and so also of WT(1) and WT-A(1)) differed by a single base (i.e., position 8 of WT(2) and WT-A(2)). Again, detection of wildtype nucleic acid sequences is desirable under some circumstances, but undesirable under other circumstances.

The 23S ribosomal nucleic acid variant identified as JP-nvCT C1522T was represented by the sequence of SEQ ID NO:12, which included a binding site for a first primer. The extension product of the first primer using the JP-nvCT C1522T template contained a binding site for an opposite-strand primer used in the amplification reaction. The subregion of JP-nvCT C1522T that was detected in the probe hybridization procedures had the sequence of JP(0) (SEQ ID NO:13), or the complement thereof allowing for substitution of RNA and DNA equivalent bases. An exemplary amplicon resulting from use of the JP-nvCT C1522T template in a TMA reaction included the probe-binding sequence of JP(1) (SEQ ID NO:14). Contained within this amplification product was a first region having the sequence of JP(2) (SEQ ID NO:15), detectable using one of the “tandem” probes disclosed herein. A second region contained within JP(1), identified herein as JP(3) (SEQ ID NO:16) was not detected by the DNA probe of 346978 (SEQ ID NO:38). Probes disclosed herein that detected the JP-nvCT C1522T variant all detected sequences contained within the amplification product of JP(1). Indeed, contained within this amplification product was a first region having the sequence of JP(2) (SEQ ID NO: 15), detectable using “tandem” probes disclosed herein. As well, a second region contained within JP(1), identified as JP(3) (SEQ ID NO:16), was detectable using, for example, a probe having the sequence of 350547 (SEQ ID NO:60). In some embodiments, the sequence of JP(3) (SEQ ID NO:16) was detected using a probe having a detectable label and/or backbone that included nucleotides where sugar moieties had 2′ methoxy groups. Generally speaking, preferred probes in accordance with the disclosure are capable of generating a detectable signal following hybridization to the sequence of at least one of JP(2) (SEQ ID NO: 15) and JP(3) (SEQ ID NO:16). Notably, the sequence of JP(3) differed from the corresponding wildtype sequences of WT(3) and WT-A(3) by a single base.

The 23S ribosomal nucleic acid variant identified as FI-nvCT C1515T was represented by the sequence of SEQ ID NO:17, which included a binding site for a first primer. The extension product of the first primer using the FI-nvCT C1515T template contained a binding site for the opposite strand primer used in the amplification reaction. The subregion of FI-nvCT C1515T that was detected in the probe hybridization procedures had the sequence of FI(0) (SEQ ID NO:18), or the complement thereof allowing for substitution of RNA and DNA equivalent bases. An exemplary amplicon resulting from use of the wildtype template of FI-nvCT C1515T in a TMA reaction included the probe-binding sequence of FI(1) (SEQ ID NO: 19). Contained within this amplification product was a first region having the sequence of FI(2) (SEQ ID NO:20), detectable using one of the “tandem” probes disclosed herein. A second region contained within FI(1), identified herein as FI(3) (SEQ ID NO:21) was not detected by the DNA probe of 346978 (SEQ ID NO:38). Probes disclosed herein that detected the FI-nvCT C1515T variant all detected sequences contained within the amplification product of FI(1). Indeed, contained within this amplification product was a first region having the sequence of FI(2) (SEQ ID NO:20), detectable using “tandem” probes disclosed herein. As well, a second region contained within FI(1), identified as FI(3) (SEQ ID NO:21), was detectable using, for example, a probe having the sequence of 350547 (SEQ ID NO:60) or 350116.1 (SEQ ID NO:59). In some embodiments, the sequence of FI(3) was detected using a probe having a detectable label and/or backbone that included nucleotides where sugar moieties have 2′ methoxy groups. Generally speaking, preferred probes in accordance with the disclosure are capable of generating a detectable signal following hybridization to the sequence of at least one of FI(2) and FI(3). Notably, the sequence of FI(3) differed from the corresponding wildtype sequences of WT(3) (SEQ ID NO:6) and WT-A(3) (SEQ ID NO:11) by a single base.

The 23S ribosomal nucleic acid variant identified as US-nvCT G1526A was represented by the sequence of SEQ ID NO:22, which included a binding site for a first primer. The extension product of the first primer using the US-nvCT G1526A template contained a binding site for the opposite strand primer used in the amplification reaction. The subregion of US-nvCT G1526A that was detected in the probe hybridization procedures had the sequence of US(0) (SEQ ID NO:23), or the complement thereof allowing for substitution of RNA and DNA equivalent bases. An exemplary amplicon resulting from use of the wildtype template of US-nvCT G1526A in a TMA reaction included the probe-binding sequence of US(1) (SEQ ID NO:24). Contained within this amplification product was a first region having the sequence of US(2) (SEQ ID NO:25), detectable using one of the “tandem” probes disclosed herein. A second region contained within US(1), identified herein as US(3) (SEQ ID NO:26) was not detected by the DNA probe of 346978 (SEQ ID NO:38). Probes disclosed herein that detected the US-nvCT G1526A variant all detected sequences contained within the amplification product of US(1). Indeed, contained within this amplification product was a first region having the sequence of US(2) (SEQ ID NO:25), detectable using “tandem” probes disclosed herein. As well, a second region contained within US(1), identified as US(3) (SEQ ID NO:26), was detectable using, for example, a probe having the sequence of 350547 (SEQ ID NO:60) or 350116.1 (SEQ ID NO:59). In some embodiments, the sequence of US(3) was detected using a probe having a detectable label and/or backbone that included nucleotides where sugar moieties have 2′ methoxy groups. Generally speaking, preferred probes in accordance with the disclosure are capable of generating a detectable signal following hybridization to the sequence of at least one of US(2) and US(3). Notably, the sequence of US(3) differed from the corresponding wildtype sequences of WT(3) (SEQ ID NO:6) and WT-A(3) (SEQ ID NO: 11) by a single base.

The 23S ribosomal nucleic acid variant identified as NO-nvCT G1523A was represented by the sequence of SEQ ID NO:27, which included a binding site for a first primer. The extension product of the first primer using the NO-nvCT G1523A template contained a binding site for the opposite strand primer used in the amplification reaction. The subregion of NO-nvCT G1523A that was detected in the probe hybridization procedures had the sequence of NO(0) (SEQ ID NO:28), or the complement thereof allowing for substitution of RNA and DNA equivalent bases. An exemplary amplicon resulting from use of the wildtype template of NO-nvCT G1523A in a TMA reaction included the probe-binding sequence of NO(1) (SEQ ID NO:29). Contained within this amplification product was a first region having the sequence of NO(2) (SEQ ID NO:30), detectable using one of the “tandem” probes disclosed herein. A second region contained within NO(1), identified herein as NO(3) (SEQ ID NO:31) was not detected by the DNA probe of 346978 (SEQ ID NO:38). Probes disclosed herein that detected the NO-nvCT G1523A variant all detected sequences contained within the amplification product of NO(1). Indeed, contained within this amplification product was a first region having the sequence of NO(2) (SEQ ID NO:30), detectable using “tandem” probes disclosed herein. As well, a second region contained within NO(1), identified as NO(3) (SEQ ID NO:31), was detectable using, for example, a probe having the sequence of 350547 (SEQ ID NO:60) or 350116.1 (SEQ ID NO:59). In some embodiments, the sequence of NO(3) was detected using a probe having a detectable label and/or backbone that includes nucleotides where sugar moieties have 2′ methoxy groups. Generally speaking, preferred probes in accordance with the disclosure are capable of generating a detectable signal following hybridization to the sequence of at least one of NO(2) and NO(3). Notably, the sequence of NO(3) differed from the corresponding wildtype sequences of WT(3) (SEQ ID NO:6) and WT-A(3) (SEQ ID NO: 11) by a single base.

### Detection Strategies

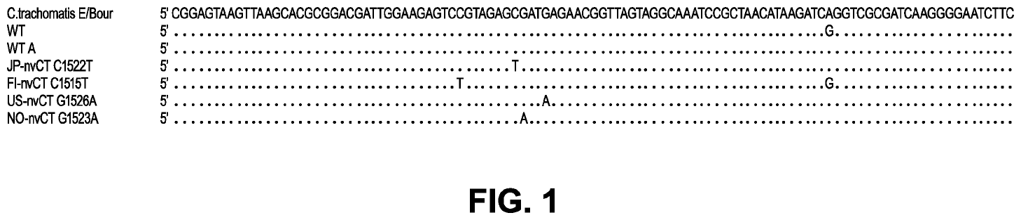

We sought to detect several closely related C. trachomatis rRNA target sequences, either singly or in combination, using a chemiluminescent detection format. FIG. 1 presents an alignment of the C. trachomatis wildtype 23S rRNA sequence (referenced in the figure as “Chlamydia trachomatis E/Bour”) (SEQ ID NO: 1) with four different variants that occur naturally around the world. The variants are identified as FI-nvCT C1515T (SEQ ID NO:17), NO-nvCT G1523A (SEQ ID NO:27), JP-nvCT C1522T (SEQ ID NO: 12), and US-nvCT G1526A (SEQ ID NO:22). Two alternative wildtype sequences presented in the figure (WT or SEQ ID NO:2; and WT-A or SEQ ID NO:7) were used for preparing in vitro transcripts (IVTs)(i.e., where T residues in the sequences were replaced by U residues in the IVTs).

Each variant differs from the wildtype sequence and from each other at only one or two base positions. In vitro transcripts corresponding to the DNA sequences shown in FIG. 1 served as templates in nucleic acid amplification (e.g., TMA) reactions that produced binding partners for the probes disclosed herein.

Three different strategies were developed to facilitate detection of either: (a) a single C. trachomatis variant without detecting wildtype sequences or any other of a particular set of variants; or (b) all variants (including the wildtype sequence). In some instances, base mismatches were deliberately introduced into the base sequences of probes. The different strategies relied on detection within C. trachomatis amplification products that were synthesized using a single pair of primers.

Example 1 describes procedures that identified hybridization probes having specificity for a particular C. trachomatis variant without detecting the wildtype sequence. Relative to a probe designed to detect an amplified wildtype C. trachomatis sequence, the approach used in this Example involved simultaneously introducing a C to T base change at position 1515, and changing the attachment of the non-nucleotide linker and AE label from position 1517 to 1515 to facilitate detection of the FI-nvCT C1515T variant sequence (SEQ ID NO: 17) without detecting the wildtype C. trachomatis sequence (e.g., SEQ ID NO:2, SEQ ID NO:3, SEQ ID NO:4, SEQ ID NO:5, or SEQ ID NO:6). The variant-specific probe in this Example can be used for epidemiological surveillance of the corresponding variant species in populations, and so is sometimes referred to as a “surveillance” probe. This approach can be adapted to detect any single variant.

### Example 1

### Identifying a Surveillance Probe Specific for a Variant Sequence

Oligonucleotide hybridization probes were synthesized and labeled with an acridinium ester using procedures familiar to those having an ordinary level of skill in the art. Base sequences of oligonucleotides used for preparation of surveillance probes are presented in Table 1. In every case, oligonucleotides were synthesized with DNA backbones and then labeled with a standard acridinium ester moiety joined to the probe backbone by an RXL non-nucleotide linker (e.g., see U.S. Pat. No. 5,585,481). Notably, the probe of 346978 (SEQ ID NO:38) was found to detect the amplified wildtype C. trachomatis sequence, but not any of the variants identified as FI-nvCT C1515T (SEQ ID NO: 17), NO-nvCT G1523A (SEQ ID NO:27), JP-nvCT C1522T (SEQ ID NO: 12), or US-nvCT G1526A (SEQ ID NO:22).

TABLE 1
Probe Oligomers

| Probe | |
|---|---|
| Identifier | Sequence |
| 346978 | AGAGTCCGT^AGAGCGATGAGAACG |
| (SEQ ID NO: 38) | |
| 350317(A) | AGAGTC^TGTAGAGCGATGAGAACG |
| (SEQ ID NO: 40) | |
| 350318 (B) | AGAGTC^TGTAGGGCGATGAGAACG |
| (SEQ ID NO: 41) | |
| 350319 (C) | AGAGTC^TGTAGAGTGATGAGAACG |
| (SEQ ID NO: 42) | |
| 350320 (D) | AGAGTCT^GTAGAGCGATGAGAACG |
| (SEQ ID NO: 43) | |
| 350321 (E) | AGAGTCT^GTAGGGCGATGAGAACG |
| (SEQ ID NO: 44) | |
| 350322 (F) | AGAGTCT^GTAGAGTGATGAGAACG |
| (SEQ ID NO: 45) | |
| 350323 (G) | AGAGTC^TGTAGAGCGATGAGA |
| (SEQ ID NO: 46) | |
| 350324 (H) | AGAGTC^TGTAGGGCGATGAGA |
| (SEQ ID NO: 47) | |
| 350325 (I) | AGAGTC^TGTAGATCGATGAGA |
| (SEQ ID NO: 48) | |
| 350326 (J) | AGAGTCT^GTAGAGCGATGAGA |
| (SEQ ID NO: 49) | |
| 350327(K) | AGAGTCT^GTAGGGCGATGAGA |
| (SEQ ID NO: 50) | |
| 350328 (L) | AGAGTCT^GTAGAGTGATGAGA |
| (SEQ ID NO: 51) | |
| 350418 (M) | AGAGTCT^GTAGAGCGATGAGAAC |
| (SEQ ID NO: 52) | |
| 350419(N) | AGAGTCT^GTAGAGCGATGAGAA |
| (SEQ ID NO: 53) | |
| 350420 (O) | GAGTCT^GTAGAGCGATGAGAACG |
| (SEQ ID NO: 54) | |
| 350421(P) | GAGTCT^GTAGAGCGATGAGAAC |
| (SEQ ID NO: 55) | |
| 350422(Q) | GAGTCT^GTAGAGCGATGAGAA |
| (SEQ ID NO: 56) | |

“^” indicates positioning of the non-nucleotide linker and AE label

Key functional parameters, including specificity and background signal production, of various acridinium ester labeled probes were assessed by procedures that involved hybridizing the probes to nucleic acid amplification products. In vitro transcripts harboring the sequence of the wildtype (SEQ ID NO:2) or the FI-nvCT C1515T variant (SEQ ID NO:17) C. trachomatis 23S rRNA were used as templates to prime transcription mediated amplification (TMA) reactions essentially as described in U.S. Pat. No. 5,514,551, the disclosure of which is incorporated by reference. Amplification reactions included either 10³ copies/ml of the variant in vitro transcript, 10⁸ copies/ml of the wildtype in vitro transcript, or the combination of 10³ copies/ml of the variant and 10⁸ copies/ml of the wildtype in vitro transcripts. Primers used in the amplification reaction were essentially as described in U.S. Pat. No. 5,514,551, the disclosure of which is incorporated by reference. Negative control amplification reactions did not receive any in vitro transcript for use as a template in the reaction. At the conclusions of the amplification procedure, each reaction tube was provided with AE-labeled probe in amounts of 1.5×10⁶ or 3×10⁶ RLU/reaction. Reaction tubes further received 100 μl of a hybridization reagent (pH 4.70) that included succinic acid, lithium lauryl sulfate, lithium hydroxide, aldrithiol-2, lithium chloride, EDTA, ethyl alcohol. Hybridization was facilitated by incubating the tubes at 62° C. for 20 minutes, followed by cooling at ambient temperatures for 5 minutes. Next, 250 μl of a selection reagent (pH 8.5) that included boric acid, sodium hydroxide, and 1% TRITON X-100 (Union Carbide Corporation; Danbury, Conn.) was added to each tube. The contents of the tubes were mixed and incubated at 62° C. for 10 minutes to hydrolyze acridinium ester labels associated with unhybridized probe, and then cooled to 23° C. Samples were then analyzed in a LEADER® HC+ luminometer (Gen-Probe Incorporated; San Diego, Calif.) equipped for automatic injection of 1 mM nitric acid and 0.1% (v/v) hydrogen peroxide, followed by injection of a solution containing 1 N sodium hydroxide. Results for the chemiluminescent reactions were measured in relative light units (RLU). All reactions were carried out in replicates of 10.

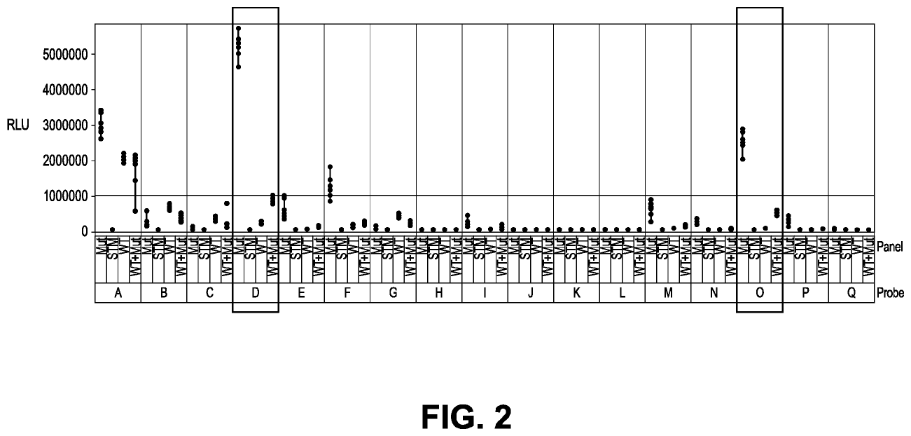

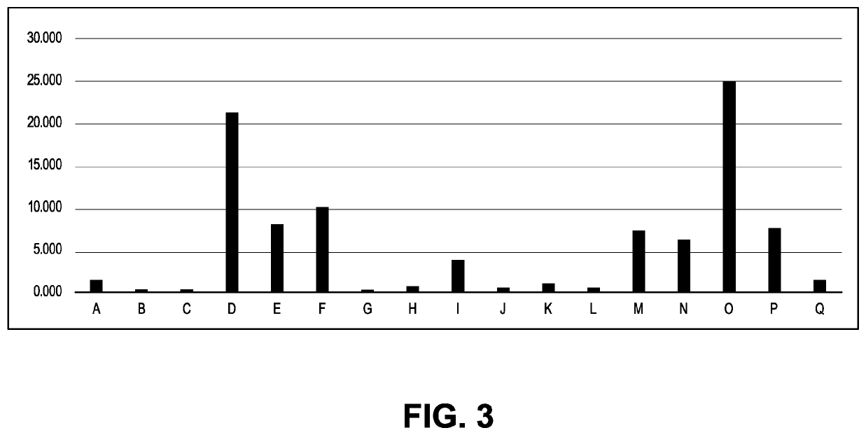

The results presented in FIG. 2 indicated that the different hybridization probes exhibited different performance characteristics. For example, probes identified in the figure as D (350320; SEQ ID NO:43), O (350420; SEQ ID NO:54), and F (350322; SEQ ID NO:45) advantageously yielded strong signals when hybridized to the variant nucleic acid sequence (“mut” in the figure), and yielded substantially weaker signals when hybridized to the wildtype (“wt”) nucleic acid sequence. FIG. 3 presents the results of processing the data from FIG. 2 to identify the relative specificity of each probe for the variant target nucleic acid. Clearly, probes D, O and F exhibited the greatest ratio of variant to wildtype signal specificity. These characteristics made these probes useful for specific detection of the FI-nvCT C1515T variant sequence (SEQ ID NO:17). Each of probes D, O, and F exhibited characteristics desirable for surveillance monitoring of the C. trachomatis variant among a population or within a geographic region. Comparison testing of probes D and O to determine limits of detection at the 95% confidence interval using a standard Probit analysis revealed that probe D was useful for detecting lower input target copy levels, possibly by as much as 5-10 fold (data not shown). The probe D 95% limit of detection using known quantities of in vitro transcripts as templates in the amplification reaction was 210 copies/ml in a urine matrix, or 231 copies/ml in specimen transport medium (STM). STM is a phosphate-buffered detergent solution which, in addition to lysing cells, protects released RNAs by inhibiting the activity of RNases that may be present in the test sample.

Certain surveillance probes useful for detecting FI-nvCT C1515T without also detecting wildtype (e.g., WT and WT-A) nucleic acid sequences can include a target-specific sequence (e.g., a “core” sequence) given by SEQ ID NO:39, or the complement thereof, allowing for substitution of RNA and DNA equivalent bases. In some embodiments, a detectable label is attached to the backbone of the surveillance probe by a non-nucleotide linker at a position between bases 6 and 7 of SEQ ID NO:39. Some preferred probes contain the sequence of SEQ ID NO:39 and include the non-nucleotide linker In some embodiments, the surveillance probe is up to 24 bases in length.

Example 2 describes two different approaches that were used to produce “duplicate” probes for detecting a variant C. trachomatis nucleic acid sequence. In the context of the present disclosure, by “duplicate” probe is meant a probe that competes with a different probe for binding to the same target sequence. A first approach involved modifying a wildtype probe by substituting a single nucleotide to achieve a complementary match to the variant target sequence. A second approach involved a backbone modification. The probes can be used independently to detect the variant sequence, but also can be used as first probes in combination with a second probe that detects wildtype C. trachomatis nucleic acid.

Example 2 describes procedures used to prepare a probe that could be used independently, or that could be used in combination with a second probe that detected wildtype sequences.

### Example 2

### Duplicate Probes Useful Alone or in Combination with Other Probes

Amplification and detection procedures essentially as described under Example 1 were followed to investigate detection of amplified wildtype (SEQ ID NO:2) and FI-nvCT C1515T variant (SEQ ID NO: 17) nucleic acid sequences using either a probe that detected the wildtype sequence, or a probe combination that further included a probe that detected the variant sequence. In this instance the probe used to detect the nucleic acid of the FI-nvCT C1515T variant had the sequence of 350078 (SEQ ID NO:57) (see Table 2). In vitro transcripts produced using template sequences of the wildtype (SEQ ID NO:2) or FI-nvCT C1515T variant (SEQ ID NO:17) served as templates in amplification reactions at input levels of 0.5 fg/reaction. Amplification reaction products were hybridized with the acridinium ester labeled probe of 346978 (SEQ ID NO:38) to detect the wildtype sequence, or hybridized with the acridinium ester labeled probe of 350078 (SEQ ID NO:57) to detect the FI-nvCT C1515T variant (SEQ ID NO:17). Probe hybridization signals indicating the presence of target sequences in the reaction mixtures were assessed as described under Example 1.

TABLE 2
Probe Oligomers

| Identifier | Sequence |
|---|---|
| 346978; DNA | AGAGTCCGT^AGAGCGATGAGAACG |
| (SEQ ID NO: 38) | |
| 350078; DNA | AGAGTCTGT^AGAGCGATGAGAACG |
| (SEQ ID NO: 57) | |

“^” indicates positioning of the non-nucleotide linker and AE label

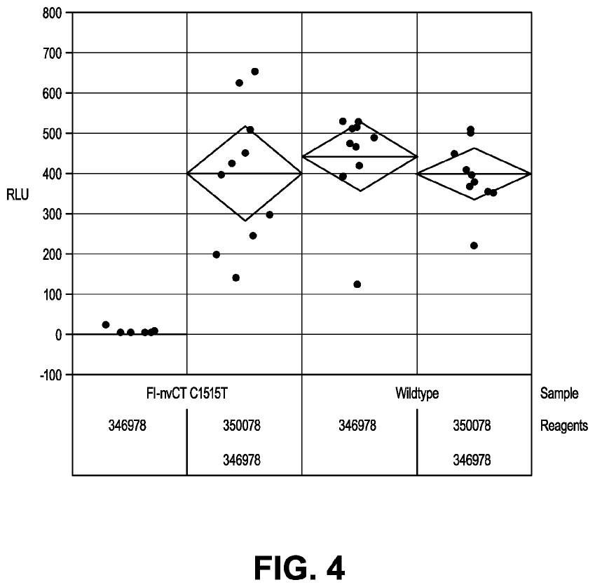

Results presented in FIG. 4 indicated that the probe of 346978 (SEQ ID NO:38) could be combined with the variant-specific probe of 350078 (SEQ ID NO:57) to produce a probe reagent capable of signaling the presence of either wildtype or variant amplified C. trachomatis target sequences. More specifically, the labeled detection probe of 346978 (SEQ ID NO:38) substantially produced a hybridization signal after hybridizing to amplification products synthesized in a TMA reaction employing an IVT corresponding to the wildtype sequence (SEQ ID NO:2), but not the FI-nvCT C1515T variant (SEQ ID NO:17) as a template. Conversely, the combination of labeled probes having the sequences of 346978 (SEQ ID NO:38) and 350078 (SEQ ID NO:57) gave strong hybridization signals following hybridization to either the amplified wildtype or FI-nvCT C1515T variant sequences. This confirmed that the two probe sequences were compatible with each other in the same reaction mixture, where the combination facilitated detection of either wildtype or FI-nvCT C1515T variant sequences. Stated differently, there was no disadvantage to combining the probes for detecting either target, even though the two probe sequences were very closely related (e.g., competitive binding being possible).

A different approach was used to create a “universal” probe for detecting both C. trachomatis wildtype and variant sequences. Here the base sequence of the probe that detected wildtype, but not variant C. trachomatis sequence was used in combination with a modified backbone analog. More specifically, universal oligonucleotide probes tested in the procedure were synthesized using nucleotide analogs having 2′-O-methyl modified pentose moieties. Base sequences of the “methoxy” probes are presented in Table 3. Again, probes used to detect the wildtype and variant target sequences were labeled with acridinium ester moieties attached via non-nucleotide linkers, and then used individually or in combination. Amplification reactions were carried out using as templates either IVTs that included sequences corresponding to FI-nvCT C1515T (SEQ ID NO:17), US-nvCT G1526A (SEQ ID NO:22), or wildtype C. trachomatis (SEQ ID NO:2). To verify specificity, one amplification reaction included a pool of lysates prepared from three organisms that were closely related to C. trachomatis. Here the candidate cross-reacting organisms were Chlamydia pneumoniae and Chlamydia psittaci. A negative control amplification reaction omitted the template nucleic acid.

TABLE 3
Probe Oligomers

| Identifier | Sequence |
|---|---|
| 350116.1; methoxy RNA | AGAGUCCGU^AGAGCGAUGAGAACG |
| (SEQ ID NO: 59) | |
| 350547; methoxy RNA | GAGUCCGUAG^AGCGAUGAGAAC |
| (SEQ ID NO: 60) | |
| 350545; methoxy RNA | GUCCGUAG^AGCGAUGAGA |
| (SEQ ID NO: 61) | |
| 350537; methoxy RNA | AGUCCGUAG^AGCGAUGAGAA |
| (SEQ ID NO: 62) | |
| 350538; methoxy RNA | AGUCCGUAGA^GCGAUGAGAA |
| (SEQ ID NO: 63) | |
| 350539; methoxy RNA | AGAGUCCGUAG^AGCGAUGAGAACG |
| (SEQ ID NO: 64) | |
| 350541; methoxy RNA | AGAGUCCGUAGA^GCGAUGAGAACG |
| (SEQ ID NO: 65) | |

“^” indicates positioning of the AE label

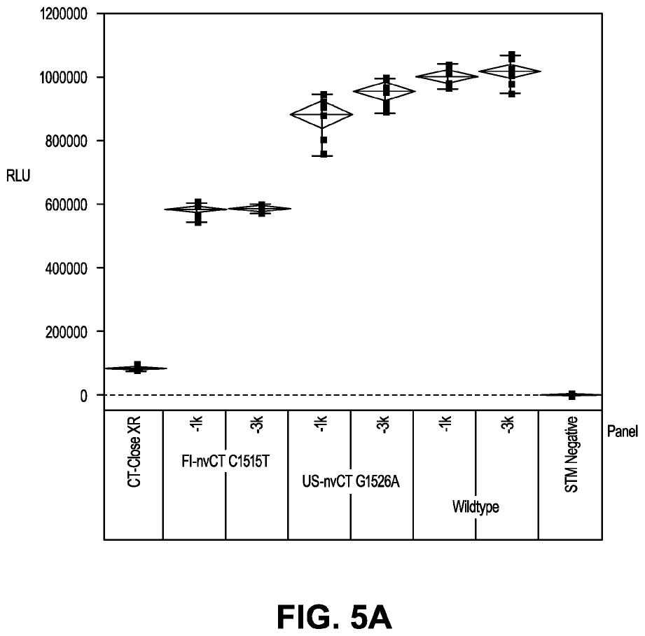

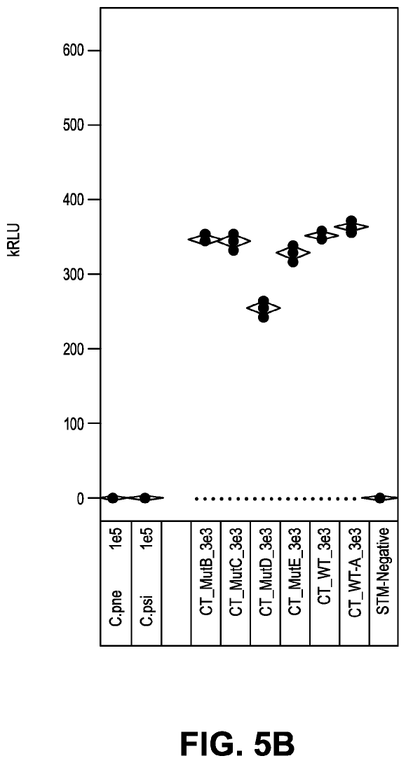

FIGS. 5A-5B present representative results obtained using one methoxy backbone probe in the absence of other probes that detect the C. trachomatis target nucleic acid. Results shown in FIG. 5A were obtained using the methoxy backbone probe of 350116.1 (SEQ ID NO:59), and indicated that substantially no hybridization signal was detected in the negative control reaction that omitted template nucleic acids. Notably, the probe of 350116.1 (SEQ ID NO:59) exhibited undesirable cross-reactive hybridization with nucleic acids of closely related, non-C. trachomatis species (“CT-Close XR” in the figure). Chemiluminescent signals well above background were observed for trials that included amplified wildtype, as well as FI-nvCT C1515T and US-nvCT G1526A variant sequences. This demonstrated that the methoxy probe was useful for detecting multiple tested target sequences. Other testing results (not shown) demonstrated that the methoxy probe could be used as a component of a probe reagent that included the AE labeled probe of 346978 (SEQ ID NO:38) for use in detecting either wildtype or variant sequences. Results shown in FIG. 5B were obtained using the methoxy probe of 350547 (SEQ ID NO:60), where this probe had a base sequence closely related to 346978 (SEQ ID NO:38), but with a different label attachment site. The probe of 350547 (SEQ ID NO:60) is a highly sensitive probe for use alone to detect a C. trachomatis amplification product, or in combination with a second probe that detects the same C. trachomatis amplicon. All of the methoxy backbone probes listed in Table 3 detected amplified nucleic acids of variant and wildtype C. trachomatis.

Certain universal duplicate probes useful for detecting all wildtype (e.g., WT and WT-A) and variant (e.g., JP-nvCT C1522T, FI-nvCT C1515T, US-nvCT G1526A, and NO-nvCT G1523A) nucleic acid sequences can include target-specific sequences complementary to at least 18 contiguous bases of the sequence given by SEQ ID NO:32, or the complement thereof, allowing for substitution of RNA and DNA equivalent bases. In some embodiments, useful universal duplicate probes include a core sequence given by SEQ ID NO:58, or the complement thereof, allowing for substitution of RNA and DNA equivalent bases. In some embodiments, a detectable label is attached to the backbone of the universal duplicate probe by a non-nucleotide linker at a position between bases 6 and 7, or between bases 8 and 9, or between bases 9 and 10 of SEQ ID NO:58. Some preferred probes contain the sequence of SEQ ID NO:58 and include the non-nucleotide linker. In some embodiments, the universal duplicate probe is up to 24 bases in length.

The following Example describes procedures used for detecting C. trachomatis variants by a method employing probes (e.g., paired sets of probes) hybridizing to different, non-overlapping sequences of amplified C. trachomatis nucleic acid. By “non-overlapping” is meant that the probes of different sequences can hybridize simultaneously to the same amplified nucleic acid strand such that hybridization of one probe does not exclude hybridization of the other. This is referred to herein as a “tandem” probe arrangement. Significantly, the tandem probes disclosed herein can be used alone (e.g., in the absence of a second C. trachomatis-specific probe hybridizing to the same amplicon), but can also be used in combination with a second probe that hybridizes to the same target nucleic acid (i.e., amplicon). Preferred second probes produce a detectable signal following hybridization to a C. trachomatis amplification product that is produced by amplification of a wildtype template, or produced by amplification of a variant template, such as any of FI-nvCT C1515T (SEQ ID NO:17), NO-nvCT G1523A (SEQ ID NO:27), JP-nvCT C1522T (SEQ ID NO:12), US-nvCT G1526A (SEQ ID NO:22), or other variant templates. The probe of 346978 (SEQ ID NO:38) served as an example second probe in this demonstration.

Example 3 describes development and testing of probes that can be used alone or in combination with second probes for detecting C. trachomatis wildtype or variant amplification products.

### Example 3

### Detection of C. trachomatis Sequences Using Tandem Probes

Detectably labeled probes that hybridized to a target sequence of C. trachomatis amplification products separate from the target sequence hybridized by the probe of 346978 (SEQ ID NO:38) were prepared by standard procedures that will be familiar to those having an ordinary level of skill in the art. The probes had the sequences presented in Table 4, and were modified to include acridinium ester labels using standard procedures. Positions of non-nucleotide linkers joining detectable labels to the probe also are given in the table. Initial testing was carried out using target nucleic acids produced as amplification products in TMA reactions primed either with the IVT corresponding to 23S ribosomal nucleic acids of wildtype C. trachomatis (SEQ ID NO:2), or with lysates from cultured bacteria of closely related species. The closely related species used in the procedure were Chlamydia pneumoniae and Chlamydia psittaci. The probe of 346978 (SEQ ID NO:38) served as a control in the procedure. The object of this initial testing was to identify probes exhibiting specific and sensitive detection of the C. trachomatis amplification product without also detecting amplification products generated from the C. pne or C. psi templates.

TABLE 4
Probe Oligomers

| Identifier | Sequence |
|---|---|
| 350440; DNA | CAAATCCGCTAACATAAG^ATCAGGTC |
| (SEQ ID NO: 67) | |
| 350441; DNA | CAAATCCGCTAACATAAGA^TCAGGTC |
| (SEQ ID NO: 68) | |
| 350442; DNA | AATCCGCTAACATAAG^ATCAGGTC |
| (SEQ ID NO: 69) | |
| 350443; DNA | AATCCGCTAACATAAGA^TCAGGTC |
| (SEQ ID NO: 70) | |
| 350502; methoxy RNA | UCCGCUAACAUAAG^AUCAGGUCGC |
| (SEQ ID NO: 71) | |
| 350506; methoxy RNA | AAUCCGCUAACAUAAG^AUCAGGUC |
| (SEQ ID NO: 72) | |
| 350507; methoxy RNA | CCGCUAACAUAAG^AUCAGGUCG |
| (SEQ ID NO: 73) | |
| 350509; methoxy RNA | CCGCUAACAUAAG^AUCAGGUCGC |
| (SEQ ID NO: 74) | |
| 350510; methoxy RNA | CCGCUAACAUAAG^AUCAGGUCGCG |
| (SEQ ID NO: 75) | |
| 350511; methoxy RNA | CCGCUAACAUAAG^AUCAGGUC |
| (SEQ ID NO: 76) | |
| 350567; methoxy RNA | CCGCUAACAUAAGA^UCAGGUCG |
| (SEQ ID NO: 77) | |
| 350566; methoxy RNA | CCGCUAACAUAAGA^UCAGGUCGCG |
| (SEQ ID NO: 78) | |
| 350565; methoxy RNA | CCGCUAACAUAAGA^UCAGGUCG |
| (SEQ ID NO: 79) | |
| 350563; methoxy RNA | UCCGCUAACAUAAG^AUCAGGUCGC |
| (SEQ ID NO: 80) | |
| 350568; methoxy RNA | GCUAACAUAAGA^UCAGGUCGCG |
| (SEQ ID NO: 81) | |
| 350570; methoxy RNA | GCTAACATAAGA^TCAGGTCGC |
| (SEQ ID NO: 82) | |
| 350571; methoxy RNA | GCTAACATAAGA^TCAGGTCGCG |
| (SEQ ID NO: 83) | |
| 350611; methoxy RNA | CGCUAACAUAAG^AUCAGGUCG |
| (SEQ ID NO: 84) | |
| 350610; methoxy RNA | CCGCUAGCAUAAG^AUCAGGUC |
| (SEQ ID NO: 85) | |
| 350609; methoxy RNA | CGCUAACAUAAG^AUCAGGUC |
| (SEQ ID NO: 86) | |
| 350601; methoxy RNA | GCUAACAUAAG^AUCAGGUCG |
| (SEQ ID NO: 87) | |
| 350600; methoxy RNA | CGCUAGCAUAAG^AUCAGGUC |
| (SEQ ID NO: 88) | |

“^” indicates positioning of the AE label

Notably, the DNA backbone probes of 350440 (SEQ ID NO:67) (350440: DNA; CT, 350441 (SEQ ID NO:68), 350442 (SEQ ID NO:69), and 350443 (SEQ ID NO:70) yielded unacceptably low sensitivity in the hybridization assays (results not shown).

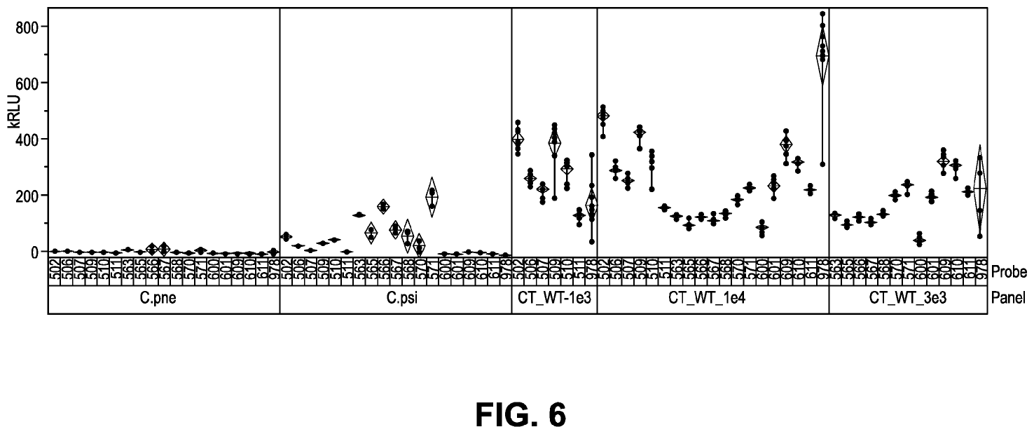

Results presented in FIG. 6 indicated that some of the probes exhibited particularly advantageous properties. As a result, we elected to explore the use of methoxy backbone probes as alternatives. Probes identified by 350507 (SEQ ID NO:73), 350511 (SEQ ID NO:76), 350600 (SEQ ID NO:88), 350601 (SEQ ID NO:87), 350609 (SEQ ID NO:86), 350610 (SEQ ID NO:85), and 350611 (SEQ ID NO:84) all exhibited very low signals when hybridized to amplification products of non-C. trachomatis species. Conversely, these same probes exhibited strong signals when hybridized to amplicons produced using the C. trachomatis IVT. This demonstrated good specificity and sensitivity for this collection of probes. Focusing on the portion of FIG. 6 showing results obtained for amplification of the wildtype IVT using an input level of either 1×10³ c/ml, 3×10³ c/ml, or 1×10⁴ c/ml, it should be clear that some probes yielded high signals (e.g., reflecting high sensitivity) with desirably narrow ranges (e.g., reflecting high precision). For example, results obtained using the probe of 350609 (SEQ ID NO:86) gave high signals with at least moderate precision while yielding advantageously low signals with the closely related Chlamydia pneumoniae and Chlamydia psittaci targets. Other probes exhibited somewhat lower hybridization signals when hybridized to wildtype target, but those signals were obtained with significantly greater precision. As well, these probes advantageously did not detect the non-C. trachomatis nucleic acid targets. More particularly, the probes of 350611 (SEQ ID NO:84), 350610 (SEQ ID NO:85), 350609 (SEQ ID NO:86), 350601 (SEQ ID NO:87), 350600 (SEQ ID NO:88), 350511 (SEQ ID NO:76), and 350507 (SEQ ID NO:73) gave very good signal strength when hybridized to wildtype target sequence, and exceedingly low signal strength when hybridized to non-C. trachomatis target nucleic acids. These probes were particularly preferred for detection of C. trachomatis with high sensitivity without detecting nucleic acids of Chlamydia pneumoniae or Chlamydia psittaci. Notably, the probe of 350611 (SEQ ID NO:84) gave exceptionally good results in this procedure.

Certain tandem probes useful for detecting all wildtype (e.g., WT and WT-A) and variant (e.g., JP-nvCT C1522T, FI-nvCT C1515T, US-nvCT G1526A, and NO-nvCT G1523A) nucleic acid sequences disclosed herein can include target-specific sequences complementary to at least 19 contiguous bases of the sequence given by SEQ ID NO:33, or the complement thereof, allowing for substitution of RNA and DNA equivalent bases. In some embodiments, useful tandem probes include a core sequence given by SEQ ID NO:66, or the complement thereof, allowing for substitution of RNA and DNA equivalent bases. In some embodiments, a detectable label is attached to the backbone of the tandem probe by a non-nucleotide linker at a position between bases 11 and 12 of SEQ ID NO:66. Some preferred probes contain the sequence of SEQ ID NO:66 and include the non-nucleotide linker. In some embodiments, the tandem probe is up to 27 bases in length.

While the present disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the disclosure requires features or combinations of features other than those expressly recited in the claims. Accordingly, the present disclosure is deemed to include all modifications and variations encompassed within the spirit and scope of the following numbered embodiments.

### NUMBERED EMBODIMENTS

Embodiment 1 is a probe reagent for detecting wildtype and variant C. trachomatis target nucleic acids, comprising:
a first oligonucleotide probe having
(i) a backbone,
(ii) a sequence of bases attached to the backbone, the sequence of bases comprising SEQ ID NO:66 or the complement thereof allowing for substitution of RNA and DNA equivalent bases, and
(iii) a label covalently attached to the backbone by a non-nucleotide linker,
   wherein the label produces a detectable signal if the first oligonucleotide probe hybridizes to a wildtype C. trachomatis nucleic acid sequence selected from the group consisting of SEQ ID NO:5 and SEQ ID NO: 10, or the complements thereof allowing for substitution of RNA and DNA equivalent bases, and
   wherein the label produces a detectable signal if the first oligonucleotide probe hybridizes to a variant C. trachomatis nucleic acid sequence selected from the group consisting of SEQ ID NO:20, SEQ ID NO:30, SEQ ID NO:15, and SEQ ID NO:25, or the complements of any of these sequences allowing for substitution of RNA and DNA equivalent bases.

Embodiment 2 is the probe reagent of embodiment 1, wherein the first oligonucleotide probe is up to 24 bases in length, and wherein the non-nucleotide linker is attached to the backbone between base positions 11 and 12 of SEQ ID NO:66.

Embodiment 3 is the probe reagent of either embodiment 1 or embodiment 2, wherein the sequence of bases attached to the backbone of the first oligonucleotide probe is selected from the group consisting of SEQ ID NO:84, SEQ ID NO:85, SEQ ID NO:86, SEQ ID NO:87, SEQ ID NO:88, SEQ ID NO:76, and SEQ ID NO:73.

Embodiment 4 if the probe reagent of either embodiment 2 or embodiment 3, wherein the sequence of bases attached to the backbone of the first oligonucleotide probe is SEQ ID NO:84 with the non-nucleotide linker being attached to the backbone between base positions 12 and 13.

Embodiment 5 is the probe reagent of either embodiment 2 or embodiment 3, wherein the sequence of bases attached to the backbone of the first oligonucleotide probe is SEQ ID NO:85 with the non-nucleotide linker being attached to the backbone between base positions 13 and 14.

Embodiment 6 is the probe reagent of either embodiment 2 or embodiment 3, wherein the sequence of bases attached to the backbone of the first oligonucleotide probe is SEQ ID NO:86 with the non-nucleotide linker being attached to the backbone between base positions 12 and 13.

Embodiment 7 is the probe reagent of either embodiment 2 or embodiment 3, wherein the sequence of bases attached to the backbone of the first oligonucleotide probe is SEQ ID NO:87 with the non-nucleotide linker being attached to the backbone between base positions 11 and 12.

Embodiment 8 is the probe reagent of either embodiment 2 or embodiment 3, wherein the sequence of bases attached to the backbone of the first oligonucleotide probe is SEQ ID NO:88 with the non-nucleotide linker being attached to the backbone between base positions 12 and 13.

Embodiment 9 is the probe reagent of either embodiment 2 or embodiment 3, wherein the sequence of bases attached to the backbone of the first oligonucleotide probe is SEQ ID NO:76 with the non-nucleotide linker being attached to the backbone between base positions 13 and 14.

Embodiment 10 is the probe reagent of either embodiment 2 or embodiment 3, wherein the sequence of bases attached to the backbone of the first oligonucleotide probe is SEQ ID NO:73 with the non-nucleotide linker being attached to the backbone between base positions 13 and 14.

Embodiment 11 is the probe reagent of any one of the preceding embodiments, wherein the label of the first oligonucleotide probe comprises a chemiluminescent label.

Embodiment 12 is the probe reagent of embodiment 11, wherein the chemiluminescent label is an acridinium ester.

Embodiment 13 is the probe reagent of any one of the preceding embodiments, wherein the backbone of the first oligonucleotide probe comprises one or more 2′-methoxy chemical groups.

Embodiment 14 is the probe reagent of any one of the preceding embodiments, further comprising a second oligonucleotide probe,

wherein the second oligonucleotide probe comprises a base sequence complementary to 23S ribosomal nucleic acid of C. trachomatis, or the complement thereof, and further comprises a label covalently attached thereto,

wherein the label of the second oligonucleotide probe produces a detectable signal if the second oligonucleotide probe is hybridized to a wildtype C. trachomatis nucleic acid sequence comprising SEQ ID NO:6, or the complement thereof allowing for substitution of RNA and DNA equivalent bases, and

wherein the label of the second oligonucleotide probe does not produce a detectable signal if the second oligonucleotide probe is hybridized to a nucleic acid sequence selected from the group consisting of SEQ ID NO:21, SEQ ID NO:31, SEQ ID NO:16, and SEQ ID NO:26, or the complements thereof allowing for substitution of RNA and DNA equivalent bases.

Embodiment 15 is the probe reagent of embodiment 14, wherein the second oligonucleotide probe comprises a DNA backbone.

Embodiment 16 is the probe reagent of either embodiment 14 or embodiment 15, wherein the label of the first oligonucleotide probe is the same as the label of the second oligonucleotide probe.

Embodiment 17 is the probe reagent of any one of embodiments 14 to 16, wherein the base sequence of the second oligonucleotide probe is SEQ ID NO:38.

Embodiment 18 is a probe reagent for detecting wildtype and variant C. trachomatis target nucleic acids, comprising:

a first oligonucleotide probe having
(i) a backbone,
(ii) a sequence of bases attached to the backbone, the sequence of bases comprising SEQ ID NO:66 or the complement thereof allowing for substitution of RNA and DNA equivalent bases, and
(iii) a label covalently attached to the backbone by a non-nucleotide linker,
   wherein the label produces a detectable signal if the oligonucleotide probe hybridizes to a target nucleic acid sequence of SEQ ID NO:2 or the complement thereof allowing for substitution of RNA and DNA equivalent bases,
   wherein the label produces a detectable signal if the oligonucleotide probe hybridizes to a target nucleic acid sequence of SEQ ID NO:7 or the complement thereof allowing for substitution of RNA and DNA equivalent bases,
   wherein the label produces a detectable signal if the oligonucleotide probe hybridizes to a target nucleic acid sequence of SEQ ID NO: 17 or the complement thereof allowing for substitution of RNA and DNA equivalent bases,
   wherein the label produces a detectable signal if the oligonucleotide probe hybridizes to a target nucleic acid sequence of SEQ ID NO:27 or the complement thereof allowing for substitution of RNA and DNA equivalent bases,
   wherein the label produces a detectable signal if the oligonucleotide probe hybridizes to a target nucleic acid sequence of SEQ ID NO: 12 or the complement thereof allowing for substitution of RNA and DNA equivalent bases, and
   wherein the label produces a detectable signal if the oligonucleotide probe hybridizes to a target nucleic acid sequence of SEQ ID NO:22 or the complement thereof allowing for substitution of RNA and DNA equivalent bases.

Embodiment 19 is the probe reagent of embodiment 18, wherein the first oligonucleotide probe is up to 24 bases in length, and wherein the non-nucleotide linker is attached to the backbone between base positions 11 and 12 of SEQ ID NO:66.

Embodiment 20 is the probe reagent of either embodiment 18 or embodiment 19, wherein the sequence of bases attached to the backbone of the first oligonucleotide probe is selected from the group consisting of SEQ ID NO:84, SEQ ID NO:85, SEQ ID NO:86, SEQ ID NO:87, SEQ ID NO:88, SEQ ID NO:76, and SEQ ID NO:73.

Embodiment 21 is the probe reagent of either embodiment 19 or embodiment 20, wherein the sequence of bases attached to the backbone of the first oligonucleotide probe is SEQ ID NO:84 with the non-nucleotide linker being attached to the backbone between base positions 12 and 13.

Embodiment 22 is the probe reagent of either embodiment 19 or embodiment 20, wherein the sequence of bases attached to the backbone of the first oligonucleotide probe is SEQ ID NO:85 with the non-nucleotide linker being attached to the backbone between base positions 13 and 14.

Embodiment 23 is the probe reagent of either embodiment 19 or embodiment 20, wherein the sequence of bases attached to the backbone of the first oligonucleotide probe is SEQ ID NO:86 with the non-nucleotide linker being attached to the backbone between base positions 12 and 13.

Embodiment 24 is the probe reagent of either embodiment 19 or embodiment 20, wherein the sequence of bases attached to the backbone of the first oligonucleotide probe is SEQ ID NO:87 with the non-nucleotide linker being attached to the backbone between base positions 11 and 12.

Embodiment 25 is the probe reagent of either embodiment 19 or embodiment 20, wherein the sequence of bases attached to the backbone of the first oligonucleotide probe is SEQ ID NO:88 with the non-nucleotide linker being attached to the backbone between base positions 12 and 13.

Embodiment 26 is the probe reagent of either embodiment 19 or embodiment 20, wherein the sequence of bases attached to the backbone of the first oligonucleotide probe is SEQ ID NO:76 with the non-nucleotide linker being attached to the backbone between base positions 13 and 14.

Embodiment 27 is the probe reagent of either embodiment 19 or embodiment 20, wherein the sequence of bases attached to the backbone of the first oligonucleotide probe is SEQ ID NO:73 with the non-nucleotide linker being attached to the backbone between base positions 13 and 14.

Embodiment 28 is the probe reagent of any one of the preceding embodiments, wherein the label of the first oligonucleotide probe comprises a chemiluminescent label.

Embodiment 29 is the probe reagent of embodiment 28, wherein the chemiluminescent label is an acridinium ester.

Embodiment 30 is the probe reagent of any one of the preceding embodiments, wherein the backbone of the first oligonucleotide probe comprises one or more 2′-methoxy chemical groups.

Embodiment 31 is the probe reagent of any one of the preceding embodiments, further comprising a second oligonucleotide probe,

wherein the second oligonucleotide probe comprises a base sequence complementary to 23S ribosomal nucleic acid of wildtype C. trachomatis, or the complement thereof, and further comprises a label covalently attached thereto,

wherein the label of the second oligonucleotide probe is positioned to produce a detectable signal if the second oligonucleotide probe is hybridized to a wildtype C. trachomatis nucleic acid sequence comprising SEQ ID NO:6 or the complement thereof allowing for substitution of RNA and DNA equivalent bases, and

wherein the label of the second oligonucleotide probe does not produce a detectable signal if the second oligonucleotide probe is hybridized to nucleic acid of any of a variant C. trachomatis nucleic acid sequence of SEQ ID NO:17, SEQ ID NO:27, SEQ ID NO:12, or SEQ ID NO:22 or the complements of these sequences allowing for substitution of RNA and DNA equivalent bases.

Embodiment 32 is the probe reagent of embodiment 30, wherein the second oligonucleotide probe comprises a DNA backbone.

Embodiment 33 is the probe reagent of either embodiment 31 or embodiment 32, wherein the label of the first oligonucleotide probe is the same as the label of the second oligonucleotide probe.

Embodiment 34 is the probe reagent of any one of embodiments 31 to 18, wherein the base sequence of the second oligonucleotide probe is SEQ ID NO:38.

Embodiment 35 is a kit for detecting a 23S ribosomal nucleic acid of wildtype and variant C. trachomatis, comprising a packaged combination of one or more vials containing:

a first oligonucleotide probe that produces a detectable signal if hybridized to the wildtype C. trachomatis sequence of SEQ ID NO:6, or the complement thereof allowing for substitution of RNA and DNA equivalent bases, but does not produce a detectable signal if hybridized to variant C. trachomatis nucleic acid sequences of any of SEQ ID NO:14, SEQ ID NO: 19, SEQ ID NO:24, and SEQ ID NO:29, or the complements thereof allowing for substitution of RNA and DNA equivalent bases; and

a second oligonucleotide probe that produces a detectable signal if hybridized to nucleic acid sequences of any of SEQ ID NO:4, SEQ ID NO:9, SEQ ID NO:14, SEQ ID NO: 19, SEQ ID NO:24, and SEQ ID NO:29, or the complements thereof allowing for substitution of RNA and DNA equivalent bases.

Embodiment 36 is the kit of embodiment 35, wherein the first oligonucleotide probe does not produce a detectable signal if hybridized to a nucleic acid comprising the sequence of any of SEQ ID NO:16, SEQ ID NO:21, SEQ ID NO:26, and SEQ ID NO:31, and wherein the second oligonucleotide probe produces a detectable signal if hybridized to a nucleic acid comprising the sequence of any of SEQ ID NO:6, SEQ ID NO:11, SEQ ID NO:16, SEQ ID NO:21, SEQ ID NO:26, and SEQ ID NO:31.

Embodiment 37 is the kit of either embodiment 35 or embodiment 36, further comprising a promoter-primer having an upstream promoter and a downstream target-hybridizing sequence,
wherein the downstream target-hybridizing sequence is complementary to 23S ribosomal nucleic acid of wildtype C. trachomatis sequences of SEQ ID NO:2 and SEQ ID NO:7, and complementary to variant C. trachomatis sequences of SEQ ID NO: 12, SEQ ID NO:17, SEQ ID NO:22, and SEQ ID NO:27, and
wherein enzymatic extension of the promoter-primer using the nucleic acid of either SEQ ID NO:2 or SEQ ID NO:7 as templates produces extension products comprising sequences complementary to each of the first and second oligonucleotide probes.

Embodiment 38 is the kit of any one of the preceding embodiments, wherein each of the first and second oligonucleotide probes comprises a backbone and a label covalently attached to the backbone by a non-nucleotide linker.

Embodiment 39 is the kit of embodiment 38, wherein the backbone of one of the first and second oligonucleotide probes comprises at least one 2′-methoxy chemical group.

Embodiment 40 is the kit of either embodiment 38 or embodiment 39, wherein the backbone of the first oligonucleotide probe comprises DNA, and wherein the backbone of the second oligonucleotide probe comprises at least one 2′-methoxy chemical group.

Embodiment 41 is the kit of any one of embodiments 38 to 40,

wherein the second oligonucleotide probe comprises a sequence of bases attached to the backbone thereof, the sequence of bases comprising SEQ ID NO:66, or the complement thereof allowing for substitution of RNA and DNA equivalent bases, and

wherein the non-nucleotide linker is attached to the backbone of the second oligonucleotide probe between base positions 11 and 12 of SEQ ID NO:66.

Embodiment 42 is the kit of embodiment 41, wherein the sequence of bases of the second oligonucleotide probe is selected from the group consisting of SEQ ID NO:84, SEQ ID NO:85, SEQ ID NO:86, SEQ ID NO:87, SEQ ID NO:88, SEQ ID NO:76, and SEQ ID NO:73.

Embodiment 43 is the kit of embodiment 42, wherein the sequence of bases attached to the backbone of the second oligonucleotide probe is SEQ ID NO:84 with the non-nucleotide linker being attached to the backbone between base positions 12 and 13.

Embodiment 44 is the kit of embodiment 42, wherein the sequence of bases attached to the backbone of the second oligonucleotide probe is SEQ ID NO:85 with the non-nucleotide linker being attached to the backbone between base positions 13 and 14.

Embodiment 45 is the kit of embodiment 42, wherein the sequence of bases attached to the backbone of the second oligonucleotide probe is SEQ ID NO:86 with the non-nucleotide linker being attached to the backbone between base positions 12 and 13.

Embodiment 46 is the kit of embodiment 42, wherein the sequence of bases attached to the backbone of the second oligonucleotide probe is SEQ ID NO:87 with the non-nucleotide linker being attached to the backbone between base positions 11 and 12.

Embodiment 47 is the kit of embodiment 42, wherein the sequence of bases attached to the backbone of the second oligonucleotide probe is SEQ ID NO:88 with the non-nucleotide linker being attached to the backbone between base positions 12 and 13.

Embodiment 48 is the kit of embodiment 42, wherein the sequence of bases attached to the backbone of the second oligonucleotide probe is SEQ ID NO:76 with the non-nucleotide linker being attached to the backbone between base positions 13 and 14.

Embodiment 49 is the kit of embodiment 42, wherein the sequence of bases attached to the backbone of the second oligonucleotide probe is SEQ ID NO:73 with the non-nucleotide linker being attached to the backbone between base positions 13 and 14.

Embodiment 50 is the kit of any one of embodiments 38 to 49, wherein the label of each of the first and second oligonucleotide probes is the same as the other.

Embodiment 51 is the kit of any one of embodiments 38 to 42, wherein the label of each of the first and second oligonucleotide probes comprises a chemiluminescent label.

Embodiment 52 is the kit of embodiment 51, wherein the chemiluminescent label attached to each of the first and second oligonucleotide probes comprises the same chemiluminescent label.

Embodiment 53 is the kit of either embodiment 51 or embodiment 52, wherein the chemiluminescent label attached to each of the first and second oligonucleotide probes comprises an acridinium ester.

Embodiment 54 is a kit for detecting a 23S ribosomal nucleic acid of wildtype and variant C. trachomatis, comprising in packaged combination:

a first oligonucleotide probe,
wherein the first oligonucleotide probe comprises a base sequence selected from the group consisting of SEQ ID NO:84, SEQ ID NO:85, SEQ ID NO:86, SEQ ID NO:87, SEQ ID NO:88, SEQ ID NO:76, SEQ ID NO:73, or the complements thereof allowing for substitution of RNA and DNA equivalent bases,
wherein the first oligonucleotide probe further comprises a label covalently attached thereto, and
wherein the label produces a detectable signal if the first oligonucleotide probe is hybridized to (i) a wildtype C. trachomatis nucleic acid complementary to either SEQ ID NO:3 or SEQ ID NO:8, allowing for substitution of RNA and DNA equivalent bases, or (ii) a variant C. trachomatis sequence complementary to any of SEQ ID NO:18, SEQ ID NO:28, SEQ ID NO:13, and SEQ ID NO:23, allowing for substitution of RNA and DNA equivalent bases; and

a promoter-primer that comprises an upstream promoter and a downstream target-hybridizing sequence,
wherein the downstream target-hybridizing sequence is complementary to 23S ribosomal nucleic acid of C. trachomatis, and
wherein enzymatic extension of the promoter-primer using 23S ribosomal nucleic acid of wildtype or variant C. trachomatis as templates produces extension products comprising sequences complementary to the first oligonucleotide probe.

Embodiment 55 is the kit of embodiment 54, wherein the first oligonucleotide probe comprises a backbone with one or more 2′-methoxy chemical groups.

Embodiment 56 is the probe reagent of either embodiment 54 or embodiment 55, wherein the base sequence of the first oligonucleotide probe is selected from the group consisting of SEQ ID NO:84, SEQ ID NO:85, SEQ ID NO:86, SEQ ID NO:87, SEQ ID NO:88, SEQ ID NO:76, and SEQ ID NO:73.

Embodiment 57 is the kit of any one of the preceding embodiments, further comprising a second oligonucleotide probe,

wherein the second oligonucleotide probe comprises a label covalently attached thereto, and

wherein the label of the second oligonucleotide probe produces a detectable signal if the second oligonucleotide probe hybridizes to a wildtype C. trachomatis nucleic acid of SEQ ID NO:3 or SEQ ID NO:8, or the complements thereof allowing for substitution of RNA and DNA equivalent bases, and

wherein the label of the second oligonucleotide probe does not produce a detectable signal if the second oligonucleotide probe is hybridized to a nucleic acid sequence selected from the group consisting of SEQ ID NO:18, SEQ ID NO:28, SEQ ID NO:13, and SEQ ID NO:23, or the complements thereof allowing for substitution of RNA and DNA equivalent bases.

Embodiment 58 is the kit of embodiment 57, wherein the label of each of the first and second oligonucleotide probes is a chemiluminescent label.

Embodiment 59 is the kit of embodiment 58, wherein the chemiluminescent label of each of the first and second oligonucleotide probes is an acridinium ester.

Embodiment 60 is the kit of embodiment 59, wherein the chemiluminescent labels of the first and second oligonucleotide probes are the same acridinium ester.

Embodiment 61 is the kit of any one of the preceding embodiments, further comprising a reverse transcriptase and an RNA polymerase.

Embodiment 62 is the kit of any one of embodiments 55 to 61, wherein the label of the first oligonucleotide probe is covalently attached to the backbone by a non-nucleotide linker.

Embodiment 63 is the kit of embodiment 62, wherein the base sequence of the first oligonucleotide probe is SEQ ID NO:84 with the non-nucleotide linker being attached to the backbone between base positions 12 and 13.

Embodiment 64 is the kit of embodiment 62, wherein the base sequence of the first oligonucleotide probe is SEQ ID NO:85 with the non-nucleotide linker being attached to the backbone between base positions 13 and 14.

Embodiment 65 is the kit of embodiment 62, wherein the base sequence of the first oligonucleotide probe is SEQ ID NO:86 with the non-nucleotide linker being attached to the backbone between base positions 12 and 13.

Embodiment 66 is the kit of embodiment 62, wherein the base sequence of the first oligonucleotide probe is SEQ ID NO:87 with the non-nucleotide linker being attached to the backbone between base positions 11 and 12.

Embodiment 67 is the kit of embodiment 62, wherein the base sequence of the first oligonucleotide probe is SEQ ID NO:88 with the non-nucleotide linker being attached to the backbone between base positions 12 and 13.

Embodiment 68 is the kit of embodiment 62, wherein the base sequence of the first oligonucleotide probe is SEQ ID NO:76 with the non-nucleotide linker being attached to the backbone between base positions 13 and 14.

Embodiment 69 is the kit of embodiment 62, wherein the base sequence of the first oligonucleotide probe is SEQ ID NO:73 with the non-nucleotide linker being attached to the backbone between base positions 13 and 14.

Embodiment 70 is a probe reagent for detecting 23S ribosomal nucleic acid of wildtype and variant C. trachomatis, comprising:

a first oligonucleotide probe having
(i) a backbone comprising one or more 2′-methoxy chemical groups,
(ii) a sequence of bases attached to the backbone, the sequence of bases comprising SEQ ID NO:58, or the complement thereof allowing for substitution of RNA and DNA equivalent bases, and
(iii) a label covalently attached to the backbone by a non-nucleotide linker either between base positions 6 and 7, base positions 8 and 9, or base positions 9 and 10 of the sequence of SEQ ID NO:58,
wherein the label of the first oligonucleotide probe produces a detectable signal if the first oligonucleotide probe hybridizes to a target nucleic acid sequence of SEQ ID NO:6, or the complement thereof allowing for substitution of RNA and DNA equivalent bases,
wherein the label of the first oligonucleotide probe produces a detectable signal if the first oligonucleotide probe hybridizes to a target nucleic acid sequence of SEQ ID NO:21, or the complement thereof allowing for substitution of RNA and DNA equivalent bases,
wherein the label of the first oligonucleotide probe produces a detectable signal if the first oligonucleotide probe hybridizes to a target nucleic acid sequence of SEQ ID NO:31, or the complement thereof allowing for substitution of RNA and DNA equivalent bases,
wherein the label of the first oligonucleotide probe produces a detectable signal if the first oligonucleotide probe hybridizes to a target nucleic acid sequence of SEQ ID NO:16, or the complement thereof allowing for substitution of RNA and DNA equivalent bases, and
wherein the label of the first oligonucleotide probe produces a detectable signal if the first oligonucleotide probe hybridizes to a target nucleic acid sequence of SEQ ID NO:26, or the complement thereof allowing for substitution of RNA and DNA equivalent bases.

Embodiment 71 is the probe reagent of embodiment 70, wherein the sequence of bases attached to the backbone of the first oligonucleotide probe is selected from the group consisting of SEQ ID NO:59 with the non-nucleotide linker attached between base positions 9 and 10, SEQ ID NO:60 with the non-nucleotide linker attached between base positions 10 and 11, SEQ ID NO:61 with the non-nucleotide linker attached between base positions 8 and 9, SEQ ID NO:62 with the non-nucleotide linker attached between base positions 9 and 10, SEQ ID NO:63 with the non-nucleotide linker attached between base positions 10 and 11, SEQ ID NO:64 with the non-nucleotide linker attached between base positions 11 and 12, and SEQ ID NO:65 with the non-nucleotide linker attached between base positions 12 and 13.

Embodiment 72 is the probe reagent of embodiment 71, wherein the sequence of bases attached to the backbone of the first oligonucleotide probe is SEQ ID NO:60.

Embodiment 73 is the probe reagent of any one of the preceding embodiments, wherein the label comprises a chemiluminescent label.

Embodiment 74 is the probe reagent of embodiment 73, wherein the chemiluminescent label is an acridinium ester.

Embodiment 75 is the probe reagent of any one of the preceding embodiments, further comprising a second oligonucleotide probe that detects 23S ribosomal nucleic acid of wildtype C. trachomatis.

Embodiment 76 is the probe reagent of embodiment 75, wherein the second oligonucleotide comprises a label that is the same as the label joined to the backbone of the first oligonucleotide probe.

Embodiment 77 is the probe reagent of any one of embodiments 70 to 76, wherein wildtype C. trachomatis nucleic acids that can be detected comprise SEQ ID NO:2 and SEQ ID NO:7, and wherein variant C. trachomatis nucleic acids that can be detected comprise SEQ ID NO:12, SEQ ID NO:17, SEQ ID NO:22, and SEQ ID NO:27.

Embodiment 78 is a probe reagent for detecting the nucleic acid of a variant C. trachomatis, comprising:

a first oligonucleotide probe having
(i) a backbone,
(ii) a sequence of bases attached to the backbone, the sequence of bases comprising SEQ ID NO:39, or the complement thereof allowing for substitution of RNA and DNA equivalent bases, and
(iii) a label covalently attached to the backbone by a non-nucleotide linker between base positions 6 and 7 of SEQ ID NO:39,
   wherein the label produces a detectable signal if the first oligonucleotide probe hybridizes to the nucleic acid sequence of SEQ ID NO:18, or the complement thereof allowing for substitution of RNA and DNA equivalent bases, and
   wherein the label does not produce the detectable signal if the first oligonucleotide probe hybridizes to the nucleic acid sequence of either SEQ ID NO:3 or SEQ ID NO:8, or the complements of these sequences allowing for substitution of RNA and DNA equivalent bases.

Embodiment 79 is the probe reagent of embodiment 78, wherein the backbone of the first oligonucleotide probe comprises DNA.

Embodiment 80 is the probe reagent of either embodiment 78 or embodiment 79,
wherein the sequence of bases of the first oligonucleotide probe comprises SEQ ID NO:39,
wherein the label produces the detectable signal if the first oligonucleotide probe hybridizes to nucleic acid complementary to the sequence of SEQ ID NO:18, allowing for substitution of RNA and DNA equivalent bases, and
wherein the label does not produce the detectable signal if the first oligonucleotide probe hybridizes to the nucleic acid sequence complementary to the sequence of either SEQ ID NO:3 or SEQ ID NO:8.

Embodiment 81 is the probe reagent of embodiment 80, wherein the base sequence of the first oligonucleotide probe is selected from the group consisting of SEQ ID NO:43, SEQ ID NO:54, and SEQ ID NO:45.

Embodiment 82 is the probe reagent of embodiment 78, further comprising a second oligonucleotide probe that produces a detectable signal if the second oligonucleotide probe hybridizes to the wildtype C. trachomatis nucleic acid sequence complementary to the sequence of either SEQ ID NO:3 or SEQ ID NO:8.

Embodiment 83 is the probe reagent of embodiment 78, wherein the label of the first oligonucleotide probe comprises a chemiluminescent label.

Embodiment 84 is the probe reagent of embodiment 83, wherein the chemiluminescent label is an acridinium ester.

Embodiment 85 is the probe reagent of any one of the preceding embodiments, wherein the base sequence of the first oligonucleotide probe is SEQ ID NO:43 with the non-nucleotide linker being attached to the backbone between base positions 7 and 8.

Embodiment 86 is the reagent of any one of embodiments 78 to 84, wherein the base sequence of the first oligonucleotide probe is SEQ ID NO:54 with the non-nucleotide linker being attached to the backbone between base positions 6 and 7.

Embodiment 87 is the reagent of any one of embodiments 78 to 84, wherein the base sequence of the first oligonucleotide probe is SEQ ID NO:45 with the non-nucleotide linker being attached to the backbone between base positions 7 and 8.

Embodiment 88 is a reaction mixture for detecting C. trachomatis nucleic acids that may be present in a test sample, comprising:

a nucleic acid amplification product produced from a 23S ribosomal nucleic acid template of a wildtype or a variant C. trachomatis; and

one or more detectably labeled hybridization probes, each of the detectably labeled hybridization probes hybridizing to the nucleic acid amplification product, at least one of the detectably labeled hybridization probes comprising 2′-methoxy nucleotide analogs, and at least one of the detectably labeled hybridization probes producing a detectable signal after hybridizing to the nucleic acid amplification product.

Embodiment 89 is the reaction mixture of embodiment 88, wherein the variant C. trachomatis nucleic acids that are detectable comprise the sequences of SEQ ID NO:17, SEQ ID NO:12, SEQ ID NO:22, and SEQ ID NO:27.

The invention has been described with reference to a number of specific examples and embodiments. Of course, a number of different embodiments of the present invention will suggest themselves to those having ordinary skill in the art upon review of the foregoing description. Thus, the true scope of the present invention is to be determined upon reference to the appended claims.

1. A probe reagent for detecting wildtype and variant C. trachomatis target nucleic acids, comprising:
a first oligonucleotide probe having
(i) a backbone,
(ii) a sequence of bases attached to the backbone, the sequence of bases comprising SEQ ID NO:66 or the complement thereof allowing for substitution of RNA and DNA equivalent bases, and
(iii) a label covalently attached to the backbone by a non-nucleotide linker,
wherein the label produces a detectable signal if the first oligonucleotide probe hybridizes to a wildtype C. trachomatis nucleic acid sequence selected from the group consisting of SEQ ID NO:5 and SEQ ID NO:10, or the complements thereof allowing for substitution of RNA and DNA equivalent bases, and
wherein the label produces a detectable signal if the first oligonucleotide probe hybridizes to a variant C. trachomatis nucleic acid sequence selected from the group consisting of SEQ ID NO:20, SEQ ID NO:30, SEQ ID NO:15, and SEQ ID NO:25, or the complements of any of these sequences allowing for substitution of RNA and DNA equivalent bases.

2. The probe reagent of claim 1, wherein the first oligonucleotide probe is up to 24 bases in length, and wherein the non-nucleotide linker is attached to the backbone between base positions 11 and 12 of SEQ ID NO:66.

3. The probe reagent of claim 1, wherein the sequence of bases attached to the backbone of the first oligonucleotide probe is selected from the group consisting of SEQ ID NO:84, SEQ ID NO:85, SEQ ID NO:86, SEQ ID NO:87, SEQ ID NO:88, SEQ ID NO:76, and SEQ ID NO:73.

4. The probe reagent of claim 2, wherein the sequence of bases attached to the backbone of the first oligonucleotide probe is SEQ ID NO:84 with the non-nucleotide linker being attached to the backbone between base positions 12 and 13.

5. The probe reagent of claim 2, wherein the sequence of bases attached to the backbone of the first oligonucleotide probe is SEQ ID NO:85 with the non-nucleotide linker being attached to the backbone between base positions 13 and 14.

6. The probe reagent of claim 2, wherein the sequence of bases attached to the backbone of the first oligonucleotide probe is SEQ ID NO:86 with the non-nucleotide linker being attached to the backbone between base positions 12 and 13.

7. The probe reagent of claim 2, wherein the sequence of bases attached to the backbone of the first oligonucleotide probe is SEQ ID NO:87 with the non-nucleotide linker being attached to the backbone between base positions 11 and 12.

8. The probe reagent of claim 2, wherein the sequence of bases attached to the backbone of the first oligonucleotide probe is SEQ ID NO:88 with the non-nucleotide linker being attached to the backbone between base positions 12 and 13.

9. The probe reagent of claim 2, wherein the sequence of bases attached to the backbone of the first oligonucleotide probe is SEQ ID NO:76 with the non-nucleotide linker being attached to the backbone between base positions 13 and 14.

10. The probe reagent of claim 2, wherein the sequence of bases attached to the backbone of the first oligonucleotide probe is SEQ ID NO:73 with the non-nucleotide linker being attached to the backbone between base positions 13 and 14.

11. The probe reagent of claim 1, wherein the label of the first oligonucleotide probe comprises a chemiluminescent label.

12. The probe reagent of claim 11, wherein the chemiluminescent label is an acridinium ester.

13. The probe reagent of claim 1, wherein the backbone of the first oligonucleotide probe comprises one or more 2′-methoxy chemical groups.

14. The probe reagent of claim 11, further comprising a second oligonucleotide probe,
wherein the second oligonucleotide probe comprises a base sequence complementary to 23S ribosomal nucleic acid of C. trachomatis, or the complement thereof, and further comprises a label covalently attached thereto,
wherein the label of the second oligonucleotide probe produces a detectable signal if the second oligonucleotide probe is hybridized to a wildtype C. trachomatis nucleic acid sequence comprising SEQ ID NO:6, or the complement thereof allowing for substitution of RNA and DNA equivalent bases, and
wherein the label of the second oligonucleotide probe does not produce a detectable signal if the second oligonucleotide probe is hybridized to a nucleic acid sequence selected from the group consisting of SEQ ID NO:21, SEQ ID NO:31, SEQ ID NO:16, and SEQ ID NO:26, or the complements thereof allowing for substitution of RNA and DNA equivalent bases.

15. The probe reagent of claim 14, wherein the second oligonucleotide probe comprises a DNA backbone.

16. The probe reagent of claim 14, wherein the label of the first oligonucleotide probe is the same as the label of the second oligonucleotide probe.

17. The probe reagent of claim 16, wherein the base sequence of the second oligonucleotide probe is SEQ ID NO:38.

18. A probe reagent for detecting wildtype and variant C. trachomatis target nucleic acids, comprising:
a first oligonucleotide probe having
(i) a backbone,
(ii) a sequence of bases attached to the backbone, the sequence of bases comprising SEQ ID NO:66 or the complement thereof allowing for substitution of RNA and DNA equivalent bases, and
(iii) a label covalently attached to the backbone by a non-nucleotide linker,
wherein the label produces a detectable signal if the oligonucleotide probe hybridizes to a target nucleic acid sequence of SEQ ID NO:2 or the complement thereof allowing for substitution of RNA and DNA equivalent bases,
wherein the label produces a detectable signal if the oligonucleotide probe hybridizes to a target nucleic acid sequence of SEQ ID NO:7 or the complement thereof allowing for substitution of RNA and DNA equivalent bases,
wherein the label produces a detectable signal if the oligonucleotide probe hybridizes to a target nucleic acid sequence of SEQ ID NO:17 or the complement thereof allowing for substitution of RNA and DNA equivalent bases,
wherein the label produces a detectable signal if the oligonucleotide probe hybridizes to a target nucleic acid sequence of SEQ ID NO:27 or the complement thereof allowing for substitution of RNA and DNA equivalent bases,
wherein the label produces a detectable signal if the oligonucleotide probe hybridizes to a target nucleic acid sequence of SEQ ID NO:12 or the complement thereof allowing for substitution of RNA and DNA equivalent bases, and
wherein the label produces a detectable signal if the oligonucleotide probe hybridizes to a target nucleic acid sequence of SEQ ID NO:22 or the complement thereof allowing for substitution of RNA and DNA equivalent bases.

19-34. (canceled)

35. A kit for detecting a 23S ribosomal nucleic acid of wildtype and variant C. trachomatis, comprising a packaged combination of one or more vials containing:
a first oligonucleotide probe that produces a detectable signal if hybridized to the wildtype C. trachomatis sequence of SEQ ID NO:6, or the complement thereof allowing for substitution of RNA and DNA equivalent bases, but does not produce a detectable signal if hybridized to variant C. trachomatis nucleic acid sequences of any of SEQ ID NO:14, SEQ ID NO:19, SEQ ID NO:24, and SEQ ID NO:29, or the complements thereof allowing for substitution of RNA and DNA equivalent bases; and
a second oligonucleotide probe that produces a detectable signal if hybridized to nucleic acid sequences of any of SEQ ID NO:4, SEQ ID NO:9, SEQ ID NO:14, SEQ ID NO:19, SEQ ID NO:24, and SEQ ID NO:29, or the complements thereof allowing for substitution of RNA and DNA equivalent bases.

36. The kit of claim 35, wherein the first oligonucleotide probe does not produce a detectable signal if hybridized to a nucleic acid comprising the sequence of any of SEQ ID NO:16, SEQ ID NO:21, SEQ ID NO:26, and SEQ ID NO:31, and wherein the second oligonucleotide probe produces a detectable signal if hybridized to a nucleic acid comprising the sequence of any of SEQ ID NO:6, SEQ ID NO:11, SEQ ID NO:16, SEQ ID NO:21, SEQ ID NO:26, and SEQ ID NO:31.

37. The kit of claim 35, further comprising a promoter-primer having an upstream promoter and a downstream target-hybridizing sequence,
wherein the downstream target-hybridizing sequence is complementary to 23S ribosomal nucleic acid of wildtype C. trachomatis sequences of SEQ ID NO:2 and SEQ ID NO:7, and complementary to variant C. trachomatis sequences of SEQ ID NO:12, SEQ ID NO:17, SEQ ID NO:22, and SEQ ID NO:27, and
wherein enzymatic extension of the promoter-primer using the nucleic acid of either SEQ ID NO:2 or SEQ ID NO:7 as templates produces extension products comprising sequences complementary to each of the first and second oligonucleotide probes.

38. The kit of claim 35, wherein each of the first and second oligonucleotide probes comprises a backbone and a label covalently attached to the backbone by a non-nucleotide linker.

39. The kit of claim 38, wherein the backbone of one of the first and second oligonucleotide probes comprises at least one 2′-methoxy chemical group.

40. The kit of claim 38, wherein the backbone of the first oligonucleotide probe comprises DNA, and wherein the backbone of the second oligonucleotide probe comprises at least one 2′-methoxy chemical group.

41. The kit of claim 38,
wherein the second oligonucleotide probe comprises a sequence of bases attached to the backbone thereof, the sequence of bases comprising SEQ ID NO:66, or the complement thereof allowing for substitution of RNA and DNA equivalent bases, and
wherein the non-nucleotide linker is attached to the backbone of the second oligonucleotide probe between base positions 11 and 12 of SEQ ID NO:66.

42. The kit of claim 41, wherein the sequence of bases of the second oligonucleotide probe is selected from the group consisting of SEQ ID NO:84, SEQ ID NO:85, SEQ ID NO:86, SEQ ID NO:87, SEQ ID NO:88, SEQ ID NO:76, and SEQ ID NO:73.

43. The kit of claim 42, wherein the sequence of bases attached to the backbone of the first oligonucleotide probe is SEQ ID NO:84 with the non-nucleotide linker being attached to the backbone between base positions 12 and 13.

44. The kit of claim 42, wherein the sequence of bases attached to the backbone of the first oligonucleotide probe is SEQ ID NO:85 with the non-nucleotide linker being attached to the backbone between base positions 13 and 14.

45. The kit of claim 42, wherein the sequence of bases attached to the backbone of the first oligonucleotide probe is SEQ ID NO:86 with the non-nucleotide linker being attached to the backbone between base positions 12 and 13.

46. The kit of claim 42, wherein the sequence of bases attached to the backbone of the first oligonucleotide probe is SEQ ID NO:87 with the non-nucleotide linker being attached to the backbone between base positions 11 and 12.

47. The kit of claim 42, wherein the sequence of bases attached to the backbone of the first oligonucleotide probe is SEQ ID NO:88 with the non-nucleotide linker being attached to the backbone between base positions 12 and 13.

48. The kit of claim 42, wherein the sequence of bases attached to the backbone of the first oligonucleotide probe is SEQ ID NO:76 with the non-nucleotide linker being attached to the backbone between base positions 13 and 14.

49. The kit of claim 42, wherein the sequence of bases attached to the backbone of the first oligonucleotide probe is SEQ ID NO:73 with the non-nucleotide linker being attached to the backbone between base positions 13 and 14.

50. The kit of claim 38, wherein the label of each of the first and second oligonucleotide probes is the same as the other.

51. The kit of claim 38, wherein the label of each of the first and second oligonucleotide probes comprises a chemiluminescent label.

52. The kit of claim 51, wherein the chemiluminescent label attached to each of the first and second oligonucleotide probes comprises the same chemiluminescent label.

53. The kit of claim 51, wherein the chemiluminescent label attached to each of the first and second oligonucleotide probes comprises an acridinium ester.

54. A kit for detecting a 23S ribosomal nucleic acid of wildtype and variant C. trachomatis, comprising in packaged combination:
a first oligonucleotide probe,
wherein the first oligonucleotide probe comprises a base sequence selected from the group consisting of SEQ ID NO:84, SEQ ID NO:85, SEQ ID NO:86, SEQ ID NO:87, SEQ ID NO:88, SEQ ID NO:76, SEQ ID NO:73, or the complements thereof allowing for substitution of RNA and DNA equivalent bases,
wherein the first oligonucleotide probe further comprises a label covalently attached thereto, and
wherein the label produces a detectable signal if the first oligonucleotide probe is hybridized to (i) a wildtype C. trachomatis nucleic acid complementary to either SEQ ID NO:3 or SEQ ID NO:8, allowing for substitution of RNA and DNA equivalent bases, or (ii) a variant C. trachomatis sequence complementary to any of SEQ ID NO:18, SEQ ID NO:28, SEQ ID NO:13, and SEQ ID NO:23, allowing for substitution of RNA and DNA equivalent bases; and
a promoter-primer that comprises an upstream promoter and a downstream target-hybridizing sequence,
wherein the downstream target-hybridizing sequence is complementary to 23S ribosomal nucleic acid of C. trachomatis, and
wherein enzymatic extension of the promoter-primer using 23S ribosomal nucleic acid of wildtype or variant C. trachomatis as templates produces extension products comprising sequences complementary to the first oligonucleotide probe.

55. The kit of claim 54, wherein the first oligonucleotide probe comprises a backbone with one or more 2′-methoxy chemical groups.

56. The kit of claim 55, wherein the base sequence of the first oligonucleotide probe is selected from the group consisting of SEQ ID NO:84, SEQ ID NO:85, SEQ ID NO:86, SEQ ID NO:87, SEQ ID NO:88, SEQ ID NO:76, and SEQ ID NO:73.

57. The kit of claim 54, further comprising a second oligonucleotide probe,
wherein the second oligonucleotide probe comprises a label covalently attached thereto, and
wherein the label of the second oligonucleotide probe produces a detectable signal if the second oligonucleotide probe hybridizes to a wildtype C. trachomatis nucleic acid of SEQ ID NO:3 or SEQ ID NO:8, or the complements thereof allowing for substitution of RNA and DNA equivalent bases, and
wherein the label of the second oligonucleotide probe does not produce a detectable signal if the second oligonucleotide probe is hybridized to a nucleic acid sequence selected from the group consisting of SEQ ID NO:18, SEQ ID NO:28, SEQ ID NO:13, and SEQ ID NO:23, or the complements thereof allowing for substitution of RNA and DNA equivalent bases.

58. The kit of claim 57, wherein the label of each of the first and second oligonucleotide probes is a chemiluminescent label.

59. The kit of claim 58, wherein the chemiluminescent label of each of the first and second oligonucleotide probes is an acridinium ester.

60. The kit of claim 59, wherein the chemiluminescent labels of the first and second oligonucleotide probes are the same acridinium ester.

61. The kit of claim 54, further comprising a reverse transcriptase and an RNA polymerase.

62. The kit of claim 55, wherein the label of the first oligonucleotide probe is covalently attached to the backbone by a non-nucleotide linker.

63. The kit of claim 62, wherein the base sequence of the first oligonucleotide probe is SEQ ID NO:84 with the non-nucleotide linker being attached to the backbone between base positions 12 and 13.

64. The kit of claim 62, wherein the base sequence of the first oligonucleotide probe is SEQ ID NO:85 with the non-nucleotide linker being attached to the backbone between base positions 13 and 14.

65. The kit of claim 62, wherein the base sequence of the first oligonucleotide probe is SEQ ID NO:86 with the non-nucleotide linker being attached to the backbone between base positions 12 and 13.

66. The kit of claim 62, wherein the base sequence of the first oligonucleotide probe is SEQ ID NO:87 with the non-nucleotide linker being attached to the backbone between base positions 11 and 12.

67. The kit of claim 62, wherein the base sequence of the first oligonucleotide probe is SEQ ID NO:88 with the non-nucleotide linker being attached to the backbone between base positions 12 and 13.

68. The kit of claim 62, wherein the base sequence of the first oligonucleotide probe is SEQ ID NO:76 with the non-nucleotide linker being attached to the backbone between base positions 13 and 14.

69. The kit of claim 62, wherein the base sequence of the first oligonucleotide probe is SEQ ID NO:73 with the non-nucleotide linker being attached to the backbone between base positions 13 and 14.

70. A probe reagent for detecting 23S ribosomal nucleic acid of wildtype and variant C. trachomatis, comprising:
a first oligonucleotide probe having
(i) a backbone comprising one or more 2′-methoxy chemical groups,
(ii) a sequence of bases attached to the backbone, the sequence of bases comprising SEQ ID NO:58, or the complement thereof allowing for substitution of RNA and DNA equivalent bases, and
(iii) a label covalently attached to the backbone by a non-nucleotide linker either between base positions 6 and 7, base positions 8 and 9, or base positions 9 and 10 of the sequence of SEQ ID NO:58,
wherein the label of the first oligonucleotide probe produces a detectable signal if the first oligonucleotide probe hybridizes to a target nucleic acid sequence of SEQ ID NO:6, or the complement thereof allowing for substitution of RNA and DNA equivalent bases,
wherein the label of the first oligonucleotide probe produces a detectable signal if the first oligonucleotide probe hybridizes to a target nucleic acid sequence of SEQ ID NO:21, or the complement thereof allowing for substitution of RNA and DNA equivalent bases,
wherein the label of the first oligonucleotide probe produces a detectable signal if the first oligonucleotide probe hybridizes to a target nucleic acid sequence of SEQ ID NO:31, or the complement thereof allowing for substitution of RNA and DNA equivalent bases,
wherein the label of the first oligonucleotide probe produces a detectable signal if the first oligonucleotide probe hybridizes to a target nucleic acid sequence of SEQ ID NO:16, or the complement thereof allowing for substitution of RNA and DNA equivalent bases, and
wherein the label of the first oligonucleotide probe produces a detectable signal if the first oligonucleotide probe hybridizes to a target nucleic acid sequence of SEQ ID NO:26, or the complement thereof allowing for substitution of RNA and DNA equivalent bases.

71. The probe reagent of claim 70, wherein the sequence of bases attached to the backbone of the first oligonucleotide probe is selected from the group consisting of SEQ ID NO:59 with the non-nucleotide linker attached between base positions 9 and 10, SEQ ID NO:60 with the non-nucleotide linker attached between base positions 10 and 11, SEQ ID NO:61 with the non-nucleotide linker attached between base positions 8 and 9, SEQ ID NO:62 with the non-nucleotide linker attached between base positions 9 and 10, SEQ ID NO:63 with the non-nucleotide linker attached between base positions 10 and 11, SEQ ID NO:64 with the non-nucleotide linker attached between base positions 11 and 12, and SEQ ID NO:65 with the non-nucleotide linker attached between base positions 12 and 13.

72. The probe reagent of claim 71, wherein the sequence of bases attached to the backbone of the first oligonucleotide probe is SEQ ID NO:60.

73. The probe reagent of claim 71, wherein the label comprises a chemiluminescent label.

74. The probe reagent of claim 73, wherein the chemiluminescent label is an acridinium ester.

75. The probe reagent of claim 70, further comprising a second oligonucleotide probe that detects 23S ribosomal nucleic acid of wildtype C. trachomatis.

76. The probe reagent of claim 75, wherein the second oligonucleotide comprises a label that is the same as the label joined to the backbone of the first oligonucleotide probe.

77. The probe reagent of claim 70, wherein wildtype C. trachomatis nucleic acids that can be detected comprise SEQ ID NO:2 and SEQ ID NO:7, and wherein variant C. trachomatis nucleic acids that can be detected comprise SEQ ID NO:12, SEQ ID NO:17, SEQ ID NO:22, and SEQ ID NO:27.

78. A probe reagent for detecting the nucleic acid of a variant C. trachomatis, comprising:
a first oligonucleotide probe having
(i) a backbone,
(ii) a sequence of bases attached to the backbone, the sequence of bases comprising SEQ ID NO:39, or the complement thereof allowing for substitution of RNA and DNA equivalent bases, and
(iii) a label covalently attached to the backbone by a non-nucleotide linker between base positions 6 and 7 of SEQ ID NO:39,
wherein the label produces a detectable signal if the first oligonucleotide probe hybridizes to the nucleic acid sequence of SEQ ID NO:18, or the complement thereof allowing for substitution of RNA and DNA equivalent bases, and
wherein the label does not produce the detectable signal if the first oligonucleotide probe hybridizes to the nucleic acid sequence of either SEQ ID NO:3 or SEQ ID NO:8, or the complements of these sequences allowing for substitution of RNA and DNA equivalent bases.

79. The probe reagent of claim 78, wherein the backbone of the first oligonucleotide probe comprises DNA.

80. The probe reagent of claim 78,
wherein the sequence of bases of the first oligonucleotide probe comprises SEQ ID NO:39,
wherein the label produces the detectable signal if the first oligonucleotide probe hybridizes to nucleic acid complementary to the sequence of SEQ ID NO:18, allowing for substitution of RNA and DNA equivalent bases, and
wherein the label does not produce the detectable signal if the first oligonucleotide probe hybridizes to the nucleic acid sequence complementary to the sequence of either SEQ ID NO:3 or SEQ ID NO:8.

81. The probe reagent of claim 80, wherein the base sequence of the first oligonucleotide probe is selected from the group consisting of SEQ ID NO:43, SEQ ID NO:54, and SEQ ID NO:45.

82. The probe reagent of claim 78, further comprising a second oligonucleotide probe that produces a detectable signal if the second oligonucleotide probe hybridizes to the wildtype C. trachomatis nucleic acid sequence complementary to the sequence of either SEQ ID NO:3 or SEQ ID NO:8.

83. The probe reagent of claim 78, wherein the label of the first oligonucleotide probe comprises a chemiluminescent label.

84. The probe reagent of claim 83, wherein the chemiluminescent label is an acridinium ester.

85. The probe reagent of claim 82, wherein the base sequence of the first oligonucleotide probe is SEQ ID NO:43 with the non-nucleotide linker being attached to the backbone between base positions 7 and 8.

86. The probe reagent of claim 82, wherein the base sequence of the first oligonucleotide probe is SEQ ID NO:54 with the non-nucleotide linker being attached to the backbone between base positions 6 and 7.

87. The probe reagent of claim 82, wherein the base sequence of the first oligonucleotide probe is SEQ ID NO:45 with the non-nucleotide linker being attached to the backbone between base positions 7 and 8.

88. A reaction mixture for detecting C. trachomatis nucleic acids that may be present in a test sample, comprising:
a nucleic acid amplification product produced from a 23S ribosomal nucleic acid template of a wildtype or a variant C. trachomatis; and
one or more detectably labeled hybridization probes, each of the detectably labeled hybridization probes hybridizing to the nucleic acid amplification product, at least one of the detectably labeled hybridization probes comprising 2′-methoxy nucleotide analogs, and at least one of the detectably labeled hybridization probes producing a detectable signal after hybridizing to the nucleic acid amplification product.

89. The reaction mixture of claim 88, wherein the variant C. trachomatis nucleic acids that are detectable comprise the sequences of SEQ ID NO:17, SEQ ID NO:12, SEQ ID NO:22, and SEQ ID NO:27.


SEQUENCE LISTING (88 sequences)

SEQ ID NO: 1 - DNA, 108 bp, Chlamydia trachomatis
  misc_feature (1)..(108): C. trachomatis E/Bour target
cggagtaagttaagcacgcggacgattggaagagtccgtagagcgatgagaacggttagtaggcaaatccgctaacataagatcaggtcgcgatcaaggggaatcttc

SEQ ID NO: 2 - DNA, 108 bp, Chlamydia trachomatis
  misc_feature (1)..(108): Wildtype (WT) target/template sequence
cggagtaagttaagcacgcggacgattggaagagtccgtagagcgatgagaacggttagtaggcaaatccgctaacataagatcgggtcgcgatcaaggggaatcttc

SEQ ID NO: 3 - DNA, 62 bp, Chlamydia trachomatis
  misc_feature (1)..(62): WT(0) detected portion of the target/template sequence
agagtccgtagagcgatgagaacggttagtaggcaaatccgctaacataagatcgggtcgcg

SEQ ID NO: 4 - RNA, 62 nt, Chlamydia trachomatis
  misc_feature (1)..(62): WT(1) amplicon, shown as complement of WT(0)
  misc_feature (1)..(27): Probe binding site
  misc_feature (39)..(62): Probe binding site
cgcgacccgaucuuauguuagcggauuugccuacuaaccguucucaucgcucuacggacucu

SEQ ID NO: 5 - RNA, 27 nt, Chlamydia trachomatis
  misc_feature (1)..(27): WT(2) amplicon (tandem probe binding sequence)
cgcgacccgaucuuauguuagcggauu

SEQ ID NO: 6 - RNA, 24 nt, Chlamydia trachomatis
  misc_feature (1)..(24): WT(3) amplicon sequence
cguucucaucgcucuacggacucu

SEQ ID NO: 7 - DNA, 108 bp, Chlamydia trachomatis; identical to SEQ ID NO: 1
  misc_feature (1)..(108): Wildtype_A (WT-A) target/template

SEQ ID NO: 8 - DNA, 62 bp, Chlamydia trachomatis
  misc_feature (1)..(62): WT-A(0) detected portion of target/template sequence
agagtccgtagagcgatgagaacggttagtaggcaaatccgctaacataagatcaggtcgcg

SEQ ID NO: 9 - RNA, 62 nt, Chlamydia trachomatis
  misc_feature (1)..(62): WT-A(1) amplicon, shown as complement of WT-A(0)
  misc_feature (1)..(27): Probe binding site
  misc_feature (39)..(62): Probe binding site
cgcgaccugaucuuauguuagcggauuugccuacuaaccguucucaucgcucuacggacucu

SEQ ID NO: 10 - RNA, 27 nt, Chlamydia trachomatis
  misc_feature (1)..(27): WT-A(2) amplicon (tandem probe binding sequence)
cgcgaccugaucuuauguuagcggauu

SEQ ID NO: 11 - RNA, 24 nt, Chlamydia trachomatis
  misc_feature (1)..(24): WT-A(3) amplicon sequence
cguucucaucgcucuacggacucu

SEQ ID NO: 12 - DNA, 108 bp, Chlamydia trachomatis
  misc_feature (1)..(108): JP-nvCT C1522T target/template sequence
cggagtaagttaagcacgcggacgattggaagagtccgtagagtgatgagaacggttagtaggcaaatccgctaacataagatcaggtcgcgatcaaggggaatcttc

SEQ ID NO: 13 - DNA, 62 bp, Chlamydia trachomatis
  misc_feature (1)..(62): JP(0) detected portion of target/template sequence
agagtccgtagagtgatgagaacggttagtaggcaaatccgctaacataagatcaggtcgcg

SEQ ID NO: 14 - RNA, 62 nt, Chlamydia trachomatis
  misc_feature (1)..(62): JP(1) amplicon, shown as complement of JP(0)
  misc_feature (1)..(27): Probe binding site
  misc_feature (39)..(62): Probe binding site
cgcgaccugaucuuauguuagcggauuugccuacuaaccguucucaucacucuacggacucu

SEQ ID NO: 15 - RNA, 27 nt, Chlamydia trachomatis
  misc_feature (1)..(27): JP(2) amplicon (tandem probe binding sequence)
cgcgaccugaucuuauguuagcggauu

SEQ ID NO: 16 - RNA, 24 nt, Chlamydia trachomatis
  misc_feature (1)..(24): JP(3) amplicon JP variant
cguucucaucacucuacggacucu

SEQ ID NO: 17 - DNA, 108 bp, Chlamydia trachomatis
  misc_feature (1)..(108): FI-nvCT C1515T target/template
cggagtaagttaagcacgcggacgattggaagagtctgtagagcgatgagaacggttagtaggcaaatccgctaacataagatcaggtcgcgatcaaggggaatcttc

SEQ ID NO: 18 - DNA, 62 bp, Chlamydia trachomatis
  misc_feature (1)..(62): FI(0) detected portion of target/template sequence
agagtctgtagagcgatgagaacggttagtaggcaaatccgctaacataagatcaggtcgcg

SEQ ID NO: 19 - RNA, 62 nt, Chlamydia trachomatis
  misc_feature (1)..(62): FI(1) amplicon, shown as complement of FI(0)
  misc_feature (1)..(27): Probe binding site
  misc_feature (39)..(62): Probe binding site
cgcgaccugaucuuauguuagcggauuugccuacuaaccguucucaucgcucuacagacucu

SEQ ID NO: 20 - RNA, 27 nt, Chlamydia trachomatis
  misc_feature (1)..(27): FI(2) amplicon (tandem probe binding sequence)
cgcgaccugaucuuauguuagcggauu

SEQ ID NO: 21 - RNA, 24 nt, Chlamydia trachomatis
  misc_feature (1)..(24): FI(3) amplicon FI variant
cguucucaucgcucuacagacucu

SEQ ID NO: 22 - DNA, 108 bp, Chlamydia trachomatis
  misc_feature (1)..(108): US-nvCT G1526A target/template
cggagtaagttaagcacgcggacgattggaagagtccgtagagcgataagaacggttagtaggcaaatccgctaacataagatcaggtcgcgatcaaggggaatcttc

SEQ ID NO: 23 - DNA, 62 bp, Chlamydia trachomatis
  misc_feature (1)..(62): US(0) detected portion of target/template sequence
agagtccgtagagcgataagaacggttagtaggcaaatccgctaacataagatcaggtcgcg

SEQ ID NO: 24 - RNA, 62 nt, Chlamydia trachomatis
  misc_feature (1)..(62): US(1) amplicon, shown as complement of US(0)
  misc_feature (1)..(27): Probe binding site
  misc_feature (39)..(62): Probe binding site
cgcgaccugaucuuauguuagcggauuugccuacuaaccguucuuaucgcucuacggacucu

SEQ ID NO: 25 - RNA, 27 nt, Chlamydia trachomatis
  misc_feature (1)..(27): US(2) amplicon (tandem probe binding sequence)
cgcgaccugaucuuauguuagcggauu

SEQ ID NO: 26 - RNA, 24 nt, Chlamydia trachomatis
  misc_feature (1)..(24): US(3) amplicon US variant
cguucuuaucgcucuacggacucu

SEQ ID NO: 27 - DNA, 108 bp, Chlamydia trachomatis
  misc_feature (1)..(108): NO-nvCT G1523A target/template
cggagtaagttaagcacgcggacgattggaagagtccgtagagcaatgagaacggttagtaggcaaatccgctaacataagatcaggtcgcgatcaaggggaatcttc

SEQ ID NO: 28 - DNA, 62 bp, Chlamydia trachomatis
  misc_feature (1)..(62): NO(0) detected portion of target/template sequence
agagtccgtagagcaatgagaacggttagtaggcaaatccgctaacataagatcaggtcgcg

SEQ ID NO: 29 - RNA, 62 nt, Chlamydia trachomatis
  misc_feature (1)..(62): NO(1) amplicon, shown as complement of NO(0)
  misc_feature (1)..(27): Probe binding site
  misc_feature (39)..(62): Probe binding site
cgcgaccugaucuuauguuagcggauuugccuacuaaccguucucauugcucuacggacucu

SEQ ID NO: 30 - RNA, 27 nt, Chlamydia trachomatis
  misc_feature (1)..(27): NO(2) amplicon (tandem probe binding sequence)
cgcgaccugaucuuauguuagcggauu

SEQ ID NO: 31 - RNA, 24 nt, Chlamydia trachomatis
  misc_feature (1)..(24): NO(3) amplicon NO variant
cguucucauugcucuacggacucu

SEQ ID NO: 32 - DNA, 24 bp, Chlamydia trachomatis
  misc_feature (1)..(24): DP-1 (duplicate probe domain, 24-mer)
agagtccgtagagcgatgagaacg

SEQ ID NO: 33 - DNA, 27 bp, Chlamydia trachomatis
  misc_feature (1)..(27): TP-1 (tandem probe domain, 27-mer)
aatccgctaacataagatcaggtcgcg

SEQ ID NO: 34 - DNA, 33 bp, Chlamydia trachomatis
  misc_feature (1)..(33): Non-T7 primer
cggagtaagttaagcacgcggacgattggaaga

SEQ ID NO: 35 - DNA, 22 bp, Chlamydia trachomatis
  misc_feature (1)..(22): Target-binding sequence of T7 promoter-primer
cccgaagattccccttgatcgc

SEQ ID NO: 36 - DNA, 27 bp, Phage T7
  promoter (1)..(27)
aatttaatacgactcactatagggaga

SEQ ID NO: 37 - DNA, 49 bp, Artificial Sequence
  T7 promoter primer
  misc_feature (1)..(27): Phage T7 promoter sequence
  misc_feature (28)..(49): C. trachomatis sequence
aatttaatacgactcactatagggagacccgaagattccccttgatcgc

SEQ ID NO: 38 - DNA, 24 bp, Chlamydia trachomatis
  misc_feature (1)..(24): 346978
  misc_feature (9)..(10): Non-nucleotide linker and AE label attachment site
agagtccgtagagcgatgagaacg

SEQ ID NO: 39 - DNA, 12 bp, Chlamydia trachomatis
  misc_feature (1)..(12): Surveillance probe core sequence
  misc_feature (6)..(7): Non-nucleotide linker and AE label attachment site
gagtctgtagag

SEQ ID NO: 40 - DNA, 24 bp, Chlamydia trachomatis
  misc_feature (1)..(24): 350317 (A)
  misc_feature (6)..(7): Non-nucleotide linker and AE label attachment site
agagtctgtagagcgatgagaacg

SEQ ID NO: 41 - DNA, 24 bp, Chlamydia trachomatis
  misc_feature (1)..(24): 350318 (B)
  misc_feature (6)..(7): Non-nucleotide linker and AE label attachment site
agagtctgtagggcgatgagaacg

SEQ ID NO: 42 - DNA, 24 bp, Chlamydia trachomatis
  misc_feature (1)..(24): 350319 (C)
  misc_feature (6)..(7): Non-nucleotide linker and AE label attachment site
agagtctgtagagtgatgagaacg

SEQ ID NO: 43 - DNA, 24 bp, Chlamydia trachomatis
  misc_feature (1)..(24): 350320 (D)
  misc_feature (7)..(8): Non-nucleotide linker and AE label attachment site
agagtctgtagagcgatgagaacg

SEQ ID NO: 44 - DNA, 24 bp, Chlamydia trachomatis
  misc_feature (1)..(24): 350321 (E)
  misc_feature (7)..(8): Non-nucleotide linker and AE label attachment site
agagtctgtagggcgatgagaacg

SEQ ID NO: 45 - DNA, 24 bp, Chlamydia trachomatis
  misc_feature (1)..(24): 350322 (F)
  misc_feature (7)..(8): Non-nucleotide linker and AE label attachment site
agagtctgtagagtgatgagaacg

SEQ ID NO: 46 - DNA, 21 bp, Chlamydia trachomatis
  misc_feature (1)..(21): 350323 (G)
  misc_feature (6)..(7): Non-nucleotide linker and AE label attachment site
agagtctgtagagcgatgaga

SEQ ID NO: 47 - DNA, 21 bp, Chlamydia trachomatis
  misc_feature (1)..(21): 350324 (H)
  misc_feature (6)..(7): Non-nucleotide linker and AE label attachment site
agagtctgtagggcgatgaga

SEQ ID NO: 48 - DNA, 21 bp, Chlamydia trachomatis
  misc_feature (1)..(21): 350325 (I)
  misc_feature (6)..(7): Non-nucleotide linker and AE label attachment site
agagtctgtagatcgatgaga

SEQ ID NO: 49 - DNA, 21 bp, Chlamydia trachomatis
  misc_feature (1)..(21): 350326 (J)
  misc_feature (7)..(8): Non-nucleotide linker and AE label attachment site
agagtctgtagagcgatgaga

SEQ ID NO: 50 - DNA, 21 bp, Chlamydia trachomatis
  misc_feature (1)..(21): 350327 (K)
  misc_feature (7)..(8): Non-nucleotide linker and AE label attachment site
agagtctgtagggcgatgaga

SEQ ID NO: 51 - DNA, 21 bp, Chlamydia trachomatis
  misc_feature (1)..(21): 350328 (L)
  misc_feature (7)..(8): Non-nucleotide linker and AE label attachment site
agagtctgtagagtgatgaga

SEQ ID NO: 52 - DNA, 23 bp, Chlamydia trachomatis
  misc_feature (1)..(23): 350418 (M)
  misc_feature (7)..(8): Non-nucleotide linker and AE label attachment site
agagtctgtagagcgatgagaac

SEQ ID NO: 53 - DNA, 22 bp, Chlamydia trachomatis
  misc_feature (1)..(22): 350419 (N)
  misc_feature (7)..(8): Non-nucleotide linker and AE label attachment site
agagtctgtagagcgatgagaa

SEQ ID NO: 54 - DNA, 23 bp, Chlamydia trachomatis
  misc_feature (1)..(23): 350420 (O)
  misc_feature (6)..(7): Non-nucleotide linker and AE label attachment site
gagtctgtagagcgatgagaacg

SEQ ID NO: 55 - DNA, 22 bp, Chlamydia trachomatis
  misc_feature (1)..(22): 350421 (P)
  misc_feature (6)..(7): Non-nucleotide linker and AE label attachment site
gagtctgtagagcgatgagaac

SEQ ID NO: 56 - DNA, 21 bp, Chlamydia trachomatis
  misc_feature (1)..(21): 350422 (Q)
  misc_feature (6)..(7): Non-nucleotide linker and AE label attachment site
gagtctgtagagcgatgagaa

SEQ ID NO: 57 - DNA, 24 bp, Chlamydia trachomatis
  misc_feature (1)..(24): 350078
  misc_feature (9)..(10): Non-nucleotide linker and AE label attachment site
agagtctgtagagcgatgagaacg

SEQ ID NO: 58 - RNA, 18 nt, Chlamydia trachomatis
  misc_feature (1)..(18): Universal duplicate probe core sequence
  misc_feature (1)..(18): Backbone of 2′ methoxy linkages
guccguagagcgaugaga

SEQ ID NO: 59 - RNA, 24 nt, Chlamydia trachomatis
  misc_feature (1)..(24): Backbone of 2′ methoxy linkages
  misc_feature (1)..(24): 350116.1
  misc_feature (9)..(10): Non-nucleotide linker and AE label attachment site
agaguccguagagcgaugagaacg

SEQ ID NO: 60 - RNA, 22 nt, Chlamydia trachomatis
  misc_feature (1)..(22): Backbone of 2′ methoxy linkages
  misc_feature (1)..(22): 350547
  misc_feature (10)..(11): Non-nucleotide linker and AE label attachment site
gaguccguagagcgaugagaac

SEQ ID NO: 61 - RNA, 18 nt, Chlamydia trachomatis
  misc_feature (1)..(18): Backbone of 2′ methoxy linkages
  misc_feature (1)..(18): 350545
  misc_feature (8)..(9): Non-nucleotide linker and AE label attachment site
guccguagagcgaugaga

SEQ ID NO: 62 - RNA, 20 nt, Chlamydia trachomatis
  misc_feature (1)..(20): Backbone of 2′ methoxy linkages
  misc_feature (1)..(20): 350537
  misc_feature (9)..(10): Non-nucleotide linker and AE label attachment site
aguccguagagcgaugagaa

SEQ ID NO: 63 - RNA, 20 nt, Chlamydia trachomatis
  misc_feature (1)..(20): Backbone of 2′ methoxy linkages
  misc_feature (1)..(20): 350538
  misc_feature (10)..(11): Non-nucleotide linker and AE label attachment site
aguccguagagcgaugagaa

SEQ ID NO: 64 - RNA, 24 nt, Chlamydia trachomatis
  misc_feature (1)..(24): Backbone of 2′ methoxy linkages
  misc_feature (1)..(24): 350539
  misc_feature (11)..(12): Non-nucleotide linker and AE label attachment site
agaguccguagagcgaugagaacg

SEQ ID NO: 65 - RNA, 24 nt, Chlamydia trachomatis
  misc_feature (1)..(24): Backbone of 2′ methoxy linkages
  misc_feature (1)..(24): 350541
  misc_feature (12)..(13): Non-nucleotide linker and AE label attachment site
agaguccguagagcgaugagaacg

SEQ ID NO: 66 - RNA, 19 nt, Chlamydia trachomatis
  misc_feature (1)..(19): Tandem probe core sequence
  misc_feature (11)..(12): Non-nucleotide linker and AE label attachment site
gcuarcauaagaucagguc

SEQ ID NO: 67 - DNA, 26 bp, Chlamydia trachomatis
  misc_feature (1)..(26): 350440 (R)
  misc_feature (18)..(19): Non-nucleotide linker and AE label attachment site
caaatccgctaacataagatcaggtc

SEQ ID NO: 68 - DNA, 26 bp, Chlamydia trachomatis
  misc_feature (1)..(26): 350441 (S)
  misc_feature (19)..(20): Non-nucleotide linker and AE label attachment site
caaatccgctaacataagatcaggtc

SEQ ID NO: 69 - DNA, 24 bp, Chlamydia trachomatis
  misc_feature (1)..(24): 350442 (T)
  misc_feature (16)..(17): Non-nucleotide linker and AE label attachment site
aatccgctaacataagatcaggtc

SEQ ID NO: 70 - DNA, 24 bp, Chlamydia trachomatis
  misc_feature (1)..(24): 350443 (U)
  misc_feature (17)..(18): Non-nucleotide linker and AE label attachment site
aatccgctaacataagatcaggtc

SEQ ID NO: 71 - RNA, 24 nt, Chlamydia trachomatis
  misc_feature (1)..(24): 350502
  misc_feature (1)..(24): Backbone of 2′ methoxy linkages
  misc_feature (14)..(15): Non-nucleotide linker and AE label attachment site
uccgcuaacauaagaucaggucgc

SEQ ID NO: 72 - RNA, 24 nt, Chlamydia trachomatis
  misc_feature (1)..(24): 350506
  misc_feature (1)..(24): Backbone of 2′ methoxy linkages
  misc_feature (16)..(17): Non-nucleotide linker and AE label attachment site
aauccgcuaacauaagaucagguc

SEQ ID NO: 73 - RNA, 22 nt, Chlamydia trachomatis
  misc_feature (1)..(22): 350507
  misc_feature (1)..(22): Backbone of 2′ methoxy linkages
  misc_feature (13)..(14): Non-nucleotide linker and AE label attachment site
ccgcuaacauaagaucaggucg

SEQ ID NO: 74 - RNA, 23 nt, Chlamydia trachomatis
  misc_feature (1)..(23): 350509
  misc_feature (1)..(23): Backbone of 2′ methoxy linkages
  misc_feature (13)..(14): Non-nucleotide linker and AE label attachment site
ccgcuaacauaagaucaggucgc

SEQ ID NO: 75 - RNA, 24 nt, Chlamydia trachomatis
  misc_feature (1)..(24): 350510
  misc_feature (1)..(24): Backbone of 2′ methoxy linkages
  misc_feature (13)..(14): Non-nucleotide linker and AE label attachment site
ccgcuaacauaagaucaggucgcg

SEQ ID NO: 76 - RNA, 21 nt, Chlamydia trachomatis
  misc_feature (1)..(21): 350511
  misc_feature (1)..(21): Backbone of 2′ methoxy linkages
  misc_feature (13)..(14): Non-nucleotide linker and AE label attachment site
ccgcuaacauaagaucagguc

SEQ ID NO: 77 - RNA, 22 nt, Chlamydia trachomatis
  misc_feature (1)..(22): 350567
  misc_feature (1)..(22): Backbone of 2′ methoxy linkages
  misc_feature (14)..(15): Non-nucleotide linker and AE label attachment site
ccgcuaacauaagaucaggucg

SEQ ID NO: 78 - RNA, 24 nt, Chlamydia trachomatis
  misc_feature (1)..(24): 350566
  misc_feature (1)..(24): Backbone of 2′ methoxy linkages
  misc_feature (14)..(15): Non-nucleotide linker and AE label attachment site
ccgcuaacauaagaucaggucgcg

SEQ ID NO: 79 - RNA, 22 nt, Chlamydia trachomatis
  misc_feature (1)..(22): 350565
  misc_feature (1)..(22): Backbone of 2′ methoxy linkages
  misc_feature (14)..(15): Non-nucleotide linker and AE label attachment site
ccgcuaacauaagaucaggucg

SEQ ID NO: 80 - RNA, 24 nt, Chlamydia trachomatis
  misc_feature (1)..(24): 350563
  misc_feature (1)..(24): Backbone of 2′ methoxy linkages
  misc_feature (14)..(15): Non-nucleotide linker and AE label attachment site
uccgcuaacauaagaucaggucgc

SEQ ID NO: 81 - RNA, 22 nt, Chlamydia trachomatis
  misc_feature (1)..(22): 350568
  misc_feature (1)..(22): Backbone of 2′ methoxy linkages
  misc_feature (12)..(13): Non-nucleotide linker and AE label attachment site
gcuaacauaagaucaggucgcg

SEQ ID NO: 82 - DNA, 21 bp, Chlamydia trachomatis
  misc_feature (1)..(21): 350570
  misc_feature (1)..(21): Backbone of 2′ methoxy linkages
  misc_feature (12)..(13): Non-nucleotide linker and AE label attachment site
gctaacataagatcaggtcgc

SEQ ID NO: 83 - DNA, 22 bp, Chlamydia trachomatis
  misc_feature (1)..(22): 350571
  misc_feature (1)..(22): Backbone of 2′ methoxy linkages
  misc_feature (12)..(13): Non-nucleotide linker and AE label attachment site
gctaacataagatcaggtcgcg

SEQ ID NO: 84 - RNA, 21 nt, Chlamydia trachomatis
  misc_feature (1)..(21): 350611
  misc_feature (1)..(21): Backbone of 2′ methoxy linkages
  misc_feature (12)..(13): Non-nucleotide linker and AE label attachment site
cgcuaacauaagaucaggucg

SEQ ID NO: 85 - RNA, 21 nt, Chlamydia trachomatis
  misc_feature (1)..(21): 350610
  misc_feature (1)..(21): Backbone of 2′ methoxy linkages
  misc_feature (13)..(14): Non-nucleotide linker and AE label attachment site
ccgcuagcauaagaucagguc

SEQ ID NO: 86 - RNA, 20 nt, Chlamydia trachomatis
  misc_feature (1)..(20): 350609
  misc_feature (1)..(20): Backbone of 2′ methoxy linkages
  misc_feature (12)..(13): Non-nucleotide linker and AE label attachment site
cgcuaacauaagaucagguc

SEQ ID NO: 87 - RNA, 20 nt, Chlamydia trachomatis
  misc_feature (1)..(20): 350601
  misc_feature (1)..(20): Backbone of 2′ methoxy linkages
  misc_feature (11)..(12): Non-nucleotide linker and AE label attachment site
gcuaacauaagaucaggucg

SEQ ID NO: 88 - RNA, 20 nt, Chlamydia trachomatis
  misc_feature (1)..(20): 350600
  misc_feature (1)..(20): Backbone of 2′ methoxy linkages
  misc_feature (12)..(13): Non-nucleotide linker and AE label attachment site
cgcuagcauaagaucagguc